(19) United States
(12) Patent Application Publication
(10) Pub. No.: US 2026/0053482 A1
(43) Pub. Date: Feb. 26, 2026

(54) SYSTEM FOR LEFT ATRIAL APPENDAGE CLIP

(71) Applicant: EDWARDS LIFESCIENCES CORPORATION, Irvine, CA (US)

(72) Inventors: Manouchehr A. Miraki, Laguna Hills, CA (US); Bryan A. Janish, Huntington Beach, CA (US); Amy E. Munnelly, Irvine, CA (US); Nicolas Alexander Herrera, Anaheim, CA (US); Maria L. Saravia, Irvine, CA (US); Irvin John Narciso, Tustin, CA (US); Daniel James Murray, Orange, CA (US); Da-Yu Chang, Irvine, CA (US); Harvey H. Chen, Irvine, CA (US); Cindy Woo, Irvine, CA (US); Mark Van Nest, Rancho Santa Margarita, CA (US); Laura Elizabeth Wasson, Silverado, CA (US); James Ryan Head, Santa Ana, CA (US); Said Pashtoun Sadat, Temecula, CA (US); Sreekumar Ramasubramanian, Irvine, CA (US); Arti Prasad Roth, Lake Forest, CA (US); Dannette Fay Casper, Mission Viejo, CA (US); Brandon Long Liu, Irvine, CA (US); Jason Thai Le, Garden Grove, CA (US); Bernard Mulvihill, Mission Viejo, CA (US); Kenneth Yesung Chen, Santa Ana, CA (US); Sakyasingh Tripathy, San Diego, CA (US)

(21) Appl. No.: 19/373,459

(22) Filed: Oct. 29, 2025

Related U.S. Application Data
(63) Continuation of application No. PCT/US2024/027572, filed on May 3, 2024.
(60) Provisional application No. 63/500,574, filed on May 5, 2023.

(51) Int. Cl. A61B 17/00; A61B 90/00

(52) U.S. Cl. CPC A61B 17/0057; A61B 90/03; A61B 2017/00367; A61B 2017/00584; A61B 2017/00623; A61B 2017/00632; A61B 2090/034

(57) ABSTRACT

A clip may be configured to close the portion of the heart, to reduce blood flow therethrough as well as passage of clots or other undesired materials. The clip may be configured to close the left atrial appendage (LAA). The closure of the LAA may reduce the possibility of stroke or other maladies stemming from fluid flow with the LAA. Delivery systems may comprise expansion apparatuses for a left atrial appendage clip. The expansion apparatuses may include retention mechanisms for retaining the clip.

### CROSS-REFERENCE TO RELATED APPLICATIONS

This application is a continuation of International Application No. PCT/US2024/027572, filed May 3, 2024, which claims the benefit of U.S. Application No. 63/500,574, filed May 5, 2023, the entire disclosures all of which are incorporated by reference for all purposes.

### BACKGROUND

### Field

Various examples disclosed herein relate generally to clips for medical implementation. Some examples relate to clips for a left atrial appendage (LAA). Some examples relate to delivery systems for clips.

### Background

Cardiac arrhythmias are abnormal heart rhythms that can cause the heart to pump blood less effectively. Atrial fibrillation (AF) is one of the most common heart arrhythmia conditions. AF causes the left atrium to beat irregularly and reduces the efficiency of the “atrial kick” that helps to move blood into the left ventricle.

The left atrial appendage (LAA) is a muscular pouch located high on the free wall of the left atrium. The anatomy of the LAA is such that blood has a tendency to stagnate and form clots within the LAA. As blood flow is reduced with the progression of AF, the potential for clot formation increases tremendously.

Clots formed in the LAA can embolize into the bloodstream and move into the brain, where they can become lodged and eventually lead to stroke. It may be beneficial to close or occlude the LAA, to reduce the possibility of clots or other undesired materials from passing into the left atrium and into the bloodstream.

Left atrial appendage closure (also known as LAA closure or LAAC) is a minimally invasive procedure that is used to reduce the risk of stroke that comes as a result of atrial fibrillation.

### SUMMARY

Systems, apparatuses, and methods disclosed herein may be directed to clips for medical implementation, including clips for a portion of a heart. The clips may be configured to close the portion of the heart, to reduce blood flow therethrough as well as passage of clots or other undesired materials. In examples, the clips may be configured to close the left atrial appendage (LAA). The closure of the LAA may reduce the possibility of stroke or other maladies stemming from fluid flow with the LAA. In examples, the clips may be positioned exterior of the LAA, to extend over an outer surface of the LAA for closure.

Systems, apparatuses, and methods disclosed herein may be directed to delivery systems for clips. The delivery systems may comprise expansion apparatuses for a clip, which may be a left atrial appendage clip. The expansion apparatuses may include retention mechanisms for retaining the clip. A retention mechanism may be utilized for retention of the clip prior to clip positioning and release, although other uses may be provided. Various forms of retention mechanisms may be provided.

In aspects, an expansion apparatus for a left atrial appendage clip is provided. The expansion apparatus may comprise an elongate shaft having a first end portion and a second end portion. The expansion apparatus may comprise an engagement portion positioned at the first end portion of the elongate shaft and having at least two arms, the at least two arms including a first arm extending longitudinally and for sliding engagement with a first jaw of the left atrial appendage clip and a second arm extending longitudinally and for sliding engagement with a second jaw of the left atrial appendage clip. The expansion apparatus may comprise a retention mechanism configured to retain the left atrial appendage clip to the engagement portion to impede sliding release of the left atrial appendage clip from the first arm and the second arm.

In aspects, a method. The method may comprise utilizing an expansion apparatus to deploy or capture a left atrial appendage clip. The expansion apparatus may include an elongate shaft having a first end portion and a second end portion. The expansion apparatus may comprise an engagement portion positioned at the first end portion of the elongate shaft and having at least two arms, the at least two arms including a first arm extending longitudinally and for sliding engagement with a first jaw of the left atrial appendage clip and a second arm extending longitudinally and for sliding engagement with a second jaw of the left atrial appendage clip. The expansion apparatus may comprise a retention mechanism configured to retain the left atrial appendage clip to the engagement portion to impede sliding release of the left atrial appendage clip from the first arm and the second arm.

In aspects, an expansion apparatus for a left atrial appendage clip is provided. The expansion apparatus may comprise an elongate shaft having a first end portion and a second end portion. The expansion apparatus may comprise an engagement portion positioned at the first end portion of the elongate shaft and having at least two arms each extending from a base, the at least two arms including: a first arm extending longitudinally and for sliding engagement with a first jaw of the left atrial appendage clip and having a proximal end portion fixedly coupled to the base, and a second arm extending longitudinally and for sliding engagement with a second jaw of the left atrial appendage clip and having a proximal end portion pivotally coupled to the base, the second arm configured to pivot at the base to open or close the left atrial appendage clip.

In aspects, a method. The method may comprise utilizing an expansion apparatus to deploy or capture a left atrial appendage clip. The expansion apparatus may include an elongate shaft having a first end portion and a second end portion, and an engagement portion positioned at the first end portion of the elongate shaft and having at least two arms each extending from a base, the at least two arms including: a first arm extending longitudinally and for sliding engagement with a first jaw of the left atrial appendage clip and having a proximal end portion fixedly coupled to the base, and a second arm extending longitudinally and for sliding engagement with a second jaw of the left atrial appendage clip and having a proximal end portion pivotally coupled to the base, the second arm configured to pivot at the base to open or close the left atrial appendage clip.

In aspects, a clip for a portion of a heart. The clip may comprise a first jaw extending from a first end portion to a second end portion along a length of the first jaw, the first jaw including a first compression surface having a concave curvature relative to the first jaw. The clip may comprise a second jaw extending from a first end portion to a second end portion along a length of the second jaw, the second jaw including a second compression surface having a concave curvature relative to the second jaw. The clip may comprise a spring configured to force the first jaw and the second jaw together to compress the portion of the heart between the first compression surface and the second compression surface.

In aspects, a method. The method may comprise deploying a clip to close a portion of a heart. The clip may include a first jaw extending from a first end portion to a second end portion along a length of the first jaw, the first jaw including a first compression surface having a concave curvature relative to the first jaw. The clip may include a second jaw extending from a first end portion to a second end portion along a length of the second jaw, the second jaw including a second compression surface having a concave curvature relative to the second jaw. The clip may include a spring configured to force the first jaw and the second jaw together to compress the portion of the heart between the first compression surface and the second compression surface.

In aspects, a clip for a portion of a heart. The clip may comprise a first jaw extending from a first end portion to a second end portion along a length of the first jaw, the first jaw including a first compression surface comprising silicone. The clip may comprise a second jaw extending from a first end portion to a second end portion along a length of the second jaw, the second jaw including a second compression surface comprising silicone. The clip may comprise a spring configured to force the first jaw and the second jaw together to compress the portion of the heart between the first compression surface and the second compression surface.

In aspects, a method. The method may comprise deploying a clip to close a portion of a heart. The clip may include a first jaw extending from a first end portion to a second end portion along a length of the first jaw, the first jaw including a first compression surface comprising silicone. The clip may include a second jaw extending from a first end portion to a second end portion along a length of the second jaw, the second jaw including a second compression surface comprising silicone. The clip may include a spring configured to force the first jaw and the second jaw together to compress the portion of the heart between the first compression surface and the second compression surface.

In aspects, a system. The system may comprise a spacer device configured to space a first compression surface of a first jaw of a clip for a portion of a heart from a second compression surface of a second jaw of the clip such that the first compression surface and the second compression surface are retained separated from each other.

In aspects, a method. The method may comprise utilizing a spacer device to space a first compression surface of a first jaw of a clip for a portion of a heart from a second compression surface of a second jaw of the clip such that the first compression surface and the second compression surface are retained separated from each other.

In aspects, a sizer for a portion of a heart. The sizer may comprise an elongate shaft. The elongate shaft may include a central shaft portion having a first end and a second end opposite the first end. The elongate shaft may include a first end portion of the elongate shaft, the first end portion being coupled to the first end of the central shaft portion and extending from the first end of the central shaft portion at a first angle. The elongate shaft may include one or more indicators on the first end portion configured to indicate a size of the portion of the heart. The elongate shaft may include a second end portion of the elongate shaft, the second end portion being coupled to the second end of the central shaft portion and extending from the second end of the central shaft portion at a second angle that is different than the first angle. The elongate shaft may include one or more indicators on the second end portion configured to indicate a size of the portion of the heart.

In aspects, a method. The method may comprise utilizing a sizer to size a portion of a heart. The sizer may include an elongate shaft having a central shaft portion having a first end and a second end opposite the first end. The elongate shaft may include a first end portion of the elongate shaft, the first end portion being coupled to the first end of the central shaft portion and extending from the first end of the central shaft portion at a first angle. The elongate shaft may include one or more indicators on the first end portion configured to indicate a size of the portion of the heart. The elongate shaft may include a second end portion of the elongate shaft, the second end portion being coupled to the second end of the central shaft portion and extending from the second end of the central shaft portion at a second angle that is different than the first angle. The elongate shaft may include one or more indicators on the second end portion configured to indicate a size of the portion of the heart.

### BRIEF DESCRIPTION OF THE DRAWINGS

FIG. 1 shows a cross sectional schematic view of a portion of a heart.

FIG. 2A illustrates a perspective view of a clip.

FIG. 2B illustrates a perspective view of the clip shown in FIG. 2A from an opposite side of the clip than shown in FIG. 2A.

FIG. 2C illustrates a side view of the clip shown in FIG. 2A.

FIG. 2D illustrates a top view of the clip shown in FIG. 2A.

FIG. 2E illustrates a proximal end view of the clip shown in FIG. 2A.

FIG. 2F illustrates a cross sectional view of the clip shown in FIG. 2A along line 2F-2F.

FIG. 2G illustrates a cross sectional view of the clip shown in FIG. 2A along line 2G-2G.

FIG. 2H illustrates an exploded view of the clip shown in FIG. 2A.

FIG. 3 illustrates a top view of the clip shown in FIG. 2A.

FIG. 4 illustrates a side view of an expansion apparatus engaged with a clip.

FIG. 5 illustrates a side view of the engagement portion of the expansion apparatus shown in FIG. 4 engaged with a clip.

FIG. 6 illustrates an exploded view of the engagement portion of the expansion apparatus shown in FIG. 4.

FIG. 7 illustrates a cross sectional view of the engagement portion of the expansion apparatus shown in FIG. 4 engaged with a clip and in an opened configuration.

FIG. 8 illustrates a side view of the engagement portion of the expansion apparatus shown in FIG. 4 engaged with a clip and in an opened configuration.

FIG. 9 illustrates a side cross sectional view of the expansion apparatus shown in FIG. 4.

FIG. 10 illustrates a perspective cross sectional view of a handle of the expansion apparatus shown in FIG. 4.

FIG. 11 illustrates a perspective cross sectional view of a handle of the expansion apparatus shown in FIG. 4.

FIG. 12 illustrates a side view of the engagement portion of the expansion apparatus shown in FIG. 4 engaged with a clip and in a partially opened configuration.

FIG. 13 illustrates a side cross sectional view of a retention mechanism of an expansion apparatus.

FIG. 14 illustrates a side cross sectional view of a handle of the expansion apparatus shown in FIG. 13.

FIG. 15 illustrates a side cross sectional view of the retention mechanism shown in FIG. 13 having a varied position.

FIG. 16 illustrates a side cross sectional view of the retention mechanism shown in FIG. 13 in the position shown in FIG. 15.

FIG. 17 illustrates a side view of an engagement portion of an expansion apparatus with portions shown in transparency.

FIG. 18 illustrates a side view of the engagement portion of the expansion apparatus shown in FIG. 17 with portions shown in transparency.

FIG. 19 illustrates a side view of the engagement portion of the expansion apparatus shown in FIG. 17 with portions shown in transparency.

FIG. 20 illustrates a side view of a control device of the retention mechanism shown in FIG. 17.

FIG. 21 illustrates a side cross sectional view of an engagement portion.

FIG. 22 illustrates a side cross sectional view of the engagement portion shown in FIG. 21.

FIG. 23 illustrates a side cross sectional view of the engagement portion shown in FIG. 21.

FIG. 24 illustrates a top view of an engagement portion of an expansion apparatus with portions shown in transparency.

FIG. 25 illustrates a perspective view of the engagement portion shown in FIG. 24 and a clip.

FIG. 26 illustrates a top view of the engagement portion shown in FIG. 25 with portions shown in transparency.

FIG. 27 illustrates a side view of a clip.

FIG. 28 illustrates a perspective view of the clip shown in FIG. 27.

FIG. 29 illustrates a perspective view of an engagement portion of an expansion apparatus.

FIG. 30 illustrates an end perspective view of the engagement portion shown in FIG. 29.

FIG. 31 illustrates a perspective view of an arm of the expansion apparatus shown in FIG. 29.

FIG. 32 illustrates a perspective view of a barrier of the expansion apparatus shown in FIG. 29.

FIG. 33 illustrates a perspective view of a barrier of the expansion apparatus shown in FIG. 29.

FIG. 34 illustrates a perspective view of the barrier shown in FIG. 33 displaced from the position shown in FIG. 33.

FIG. 35 illustrates a side view of the engagement portion shown in FIG. 33 and a clip.

FIG. 36 illustrates a perspective view of an arm of an engagement portion of an expansion apparatus.

FIG. 37 illustrates a perspective view of an arm of an engagement portion of an expansion apparatus.

FIG. 38 illustrates a perspective view of a clip.

FIG. 39 illustrates a side view of the clip shown in FIG. 38.

FIG. 40 illustrates a perspective view of an engagement portion of an expansion apparatus.

FIG. 41 illustrates a perspective view of an arm of the engagement portion shown in FIG. 40.

FIG. 42 illustrates a perspective view of an arm of the engagement portion shown in FIG. 40.

FIG. 43 illustrates a side view of the engagement portion shown in FIG. 40 engaged with a clip.

FIG. 44 illustrates a side view of the engagement portion shown in FIG. 40 engaged with a clip, with portions shown in transparency.

FIG. 45 illustrates a side schematic view of the engagement portion shown in FIG. 40 engaged with a clip.

FIG. 46 illustrates a side view of a clip.

FIG. 47 illustrates a perspective view of an arm of an engagement portion for engaging the clip shown in FIG. 46.

FIG. 48 illustrates a side view of the engagement portion shown in FIG. 47 engaging with the clip shown in FIG. 46.

FIG. 49 illustrates a perspective view of the clip shown in FIG. 46 engaged with the engagement portion shown in FIG. 48.

FIG. 50 illustrates a side view of an expansion apparatus.

FIG. 51 illustrates a cross sectional view of the expansion apparatus shown in FIG. 50.

FIG. 52 illustrates a proximal perspective view of a handle of the expansion apparatus shown in FIG. 50 with a cover of the handle removed.

FIG. 53 illustrates a distal perspective view of a handle of the expansion apparatus shown in FIG. 50 with a cover of the handle removed.

FIG. 54 illustrates a perspective detail view of a lock mechanism for the expansion apparatus shown in FIG. 50.

FIG. 55 illustrates a perspective detail view of the lock mechanism of FIG. 54 with the lock mechanism displaced from the position shown in FIG. 54.

FIG. 56 illustrates a perspective view of a lock for a rotation mechanism for the expansion apparatus shown in FIG. 50.

FIG. 57 illustrates a lower perspective view of an engagement portion for the expansion apparatus shown in FIG. 50.

FIG. 58 illustrates an upper perspective view of an engagement portion for the expansion apparatus shown in FIG. 50.

FIG. 59 illustrates an exploded view of the engagement portion for the expansion apparatus shown in FIG. 50.

FIG. 60 illustrates a cross sectional view of the engagement portion for the expansion apparatus shown in FIG. 50.

FIG. 61 illustrates an upper perspective view of the engagement portion for the expansion apparatus shown in FIG. 50 engaged with a clip, with a portion of the engagement portion shown in transparency.

FIG. 62 illustrates a side view of a clip.

FIG. 63 illustrates an upper perspective view of the clip shown in FIG. 62.

FIG. 64 illustrates a lower perspective view of the clip shown in FIG. 62 at an opposite side than shown in FIG. 63.

FIG. 65 illustrates a top view of the clip shown in FIG. 62.

FIG. 66 illustrates an end view of the clip shown in FIG. 62.

FIG. 67 illustrates a cross sectional view of the clip shown in FIG. 63 along line 67-67.

FIG. 68 illustrates a longitudinal cross sectional view of the clip shown in FIG. 62.

FIG. 69 illustrates an assembly view of the clip shown in FIG. 62.

FIG. 70 illustrates a perspective view of a clip.

FIG. 71 illustrates a schematic view of a fabric dip coated with a silicone material.

FIG. 72 illustrates a top view of two jaws of clips.

FIG. 73 illustrates a side view of jaws of clips.

FIG. 74 illustrates a side view of clip engaged by an engagement portion of an expansion apparatus.

FIG. 75 illustrates a side view of a clip.

FIG. 76 illustrates a perspective view of a jaw of the clip shown in FIG. 75.

FIG. 77 illustrates a perspective view of a spacer device.

FIG. 78 illustrates a perspective view of the spacer device shown in FIG. 77 at an opposite side than shown in FIG. 77.

FIG. 79 illustrates a side view of a clip engaged with an engagement portion of an expansion apparatus with the spacer device shown in FIG. 77 deployed.

FIG. 80 illustrates a lower perspective view of the clip of FIG. 79 engaged with an engagement portion of an expansion apparatus with the spacer device shown in FIG. 77 deployed.

FIG. 81 illustrates a perspective view of a spacer device.

FIG. 82 illustrates a perspective view of the spacer device shown in FIG. 81 from an opposite side than shown in FIG. 81.

FIG. 83 illustrates a perspective view of the spacer device shown in FIG. 81 engaged with a clip that is engaged with an expansion apparatus.

FIG. 84 illustrates a perspective view of a spacer device engaged with a clip that is engaged with an expansion apparatus.

FIG. 85 illustrates a perspective view of a spacer device.

FIG. 86 illustrates a plan view of the spacer device of FIG. 85 shown in a flattened configuration.

FIG. 87 illustrates an end view of the spacer device of FIG. 85 positioned between jaws of a clip.

FIG. 88 illustrates a top perspective view of the spacer device of FIG. 85 engaged with jaws of a clip.

FIG. 89 illustrates a perspective view of a spacer device.

FIG. 90 illustrates a perspective view of a spacer device engaged with jaws of a clip, which is engaged with arms of an expansion apparatus.

FIG. 91 illustrates a perspective view of a spacer device.

FIG. 92 illustrates a perspective view of a spacer device upon jaws of a clip.

FIG. 93 illustrates a side schematic view of a clip positioned within a tray and engaged with a spacer device.

FIG. 94 illustrates a perspective view of a spacer device.

FIG. 95 illustrates a perspective view of a spacer device.

FIG. 96 illustrates a side view of a clip engaged with an engagement portion of an expansion apparatus, with a spacer device engaged with the engagement portion.

FIG. 97 illustrates a side view of a clip engaged with an engagement portion of an expansion apparatus, with a spacer device engaged with the engagement portion.

FIG. 98 illustrates a perspective view of a spacer device.

FIG. 99 illustrates a perspective view of a spacer device.

FIG. 100 illustrates a perspective view of the spacer devices of FIGS. 98 and 99 engaged with an engagement portion of an expansion apparatus.

FIG. 101 illustrates a perspective view of a spacer device engaged with an engagement portion of an expansion apparatus.

FIG. 102 illustrates a perspective view of a spacer device engaged with an engagement portion of an expansion apparatus.

FIG. 103 illustrates a perspective view of the spacer device shown in FIG. 102 with portions of the spacer device shown in transparency.

FIG. 104 illustrates a perspective view of a spacer device engaged with an engagement portion of an expansion apparatus.

FIG. 105 illustrates a perspective view of a spacer device.

FIG. 106 illustrates a side view of the spacer device shown in FIG. 104 engaged with an engagement portion of an expansion apparatus.

FIG. 107 illustrates a perspective view of a spacer device extending over a clip and an engagement portion of an expansion apparatus.

FIG. 108 illustrates an end view of the spacer device shown in FIG. 107 extending over a clip and an engagement portion of an expansion apparatus.

FIG. 109 illustrates a perspective cross sectional view of the spacer device shown in FIG. 107.

FIG. 110 illustrates a perspective view of a spacer device.

FIG. 111 illustrates a perspective cross sectional view of the spacer device shown in FIG. 110.

FIG. 112 illustrates a perspective view of a spacer device.

FIG. 113 illustrates a perspective cross sectional view of the spacer device shown in FIG. 112.

FIG. 114 illustrates a perspective view of a spacer device.

FIG. 115 illustrates a perspective cross sectional view of the spacer device shown in FIG. 114.

FIG. 116 illustrates a perspective view of a spacer device.

FIG. 117 illustrates a perspective view of a spacer device.

FIG. 118 illustrates a perspective view of a spacer device.

FIG. 119 illustrates a perspective view of a spacer device.

FIG. 120 illustrates a cross sectional view of the spacer device of FIG. 119 positioned upon an engagement portion of an expansion apparatus.

FIG. 121 illustrates a perspective view of a spacer device.

FIG. 122 illustrates a perspective view of the spacer device of FIG. 121 engaged with an engagement portion of an expansion apparatus.

FIG. 123 illustrates a perspective view of a spacer device.

FIG. 124 illustrates a perspective view of the spacer device of FIG. 123 engaged with an engagement portion of an expansion apparatus.

FIG. 125 illustrates a perspective view of a spacer device.

FIG. 126 illustrates a side view of a sizer.

FIG. 127 illustrates a lower perspective view of the sizer shown in FIG. 126.

FIG. 128 illustrates a perspective view of the sizer shown in FIG. 127 at an opposite side of the sizer than shown in FIG. 127.

FIG. 129 illustrates a top view of the sizer shown in FIG. 126 sizing a left atrial appendage.

### DETAILED DESCRIPTION

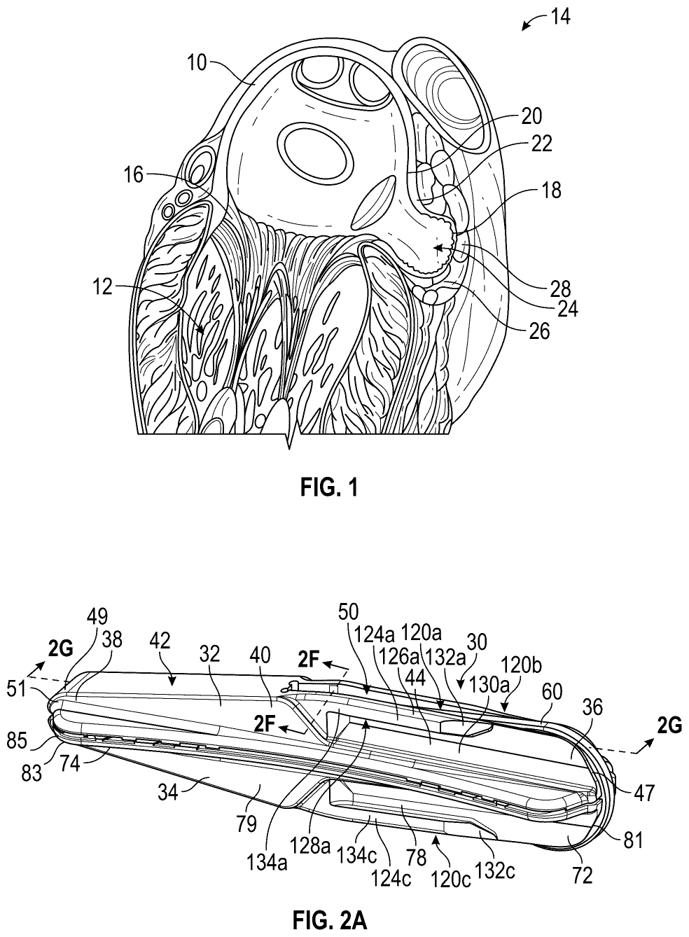

FIG. 1 illustrates a cross sectional view of a left atrium 10 and a left ventricle 12 of an individual's heart 14. The left atrium 10 is configured to fill with blood to pass into the left ventricle 12 via the mitral valve 16 during the cardiac cycle. The left atrial appendage (LAA) 18 protrudes from the outer wall 20 of the left atrium 10 and includes an ostium 22 and a cavity 24 extending from the ostium 22. A LAA wall 26 may surround the cavity 24 and may form an outer surface 28 of the LAA 18. The LAA 18 has a pouch shape extending from the left atrium 10. The cavity 24 may be configured to fill with blood, allowing the LAA to serve as a decompression chamber during systole and when pressure otherwise increases in the left atrium 10.

In certain individuals, blood may stagnate and form clots within the LAA 18. Clots or other undesired materials stemming from the LAA 18 may travel into the bloodstream, producing a variety of maladies including strokes. It may thus be beneficial to close the LAA 18, to reduce the possibility of clots or other undesired material from producing such maladies.

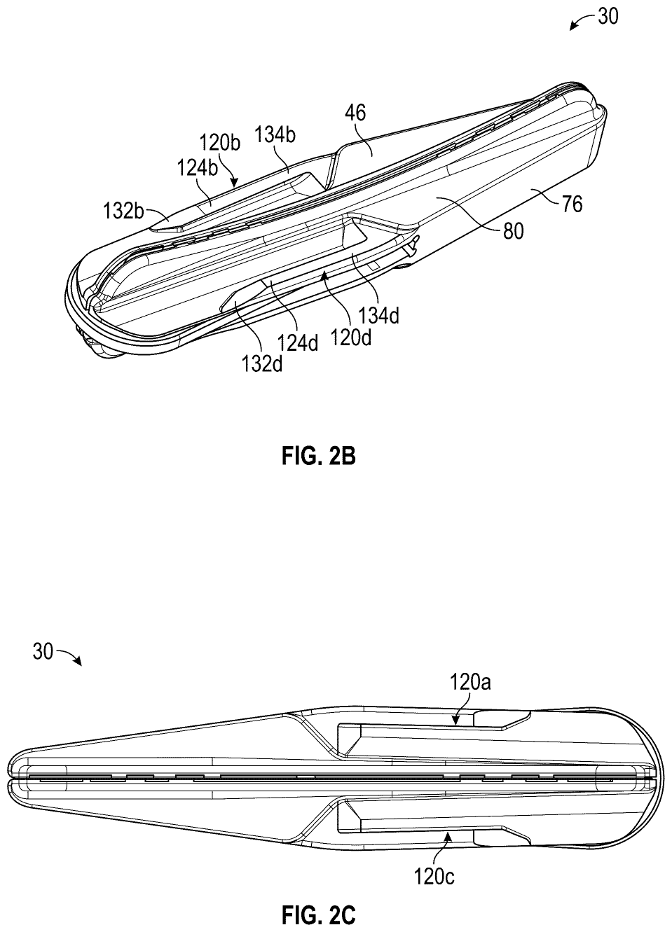

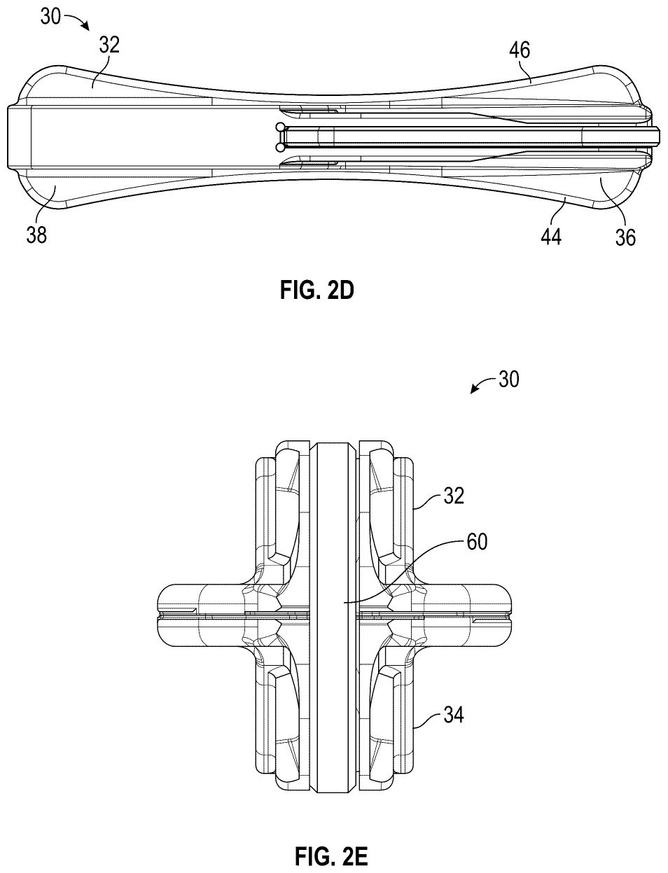

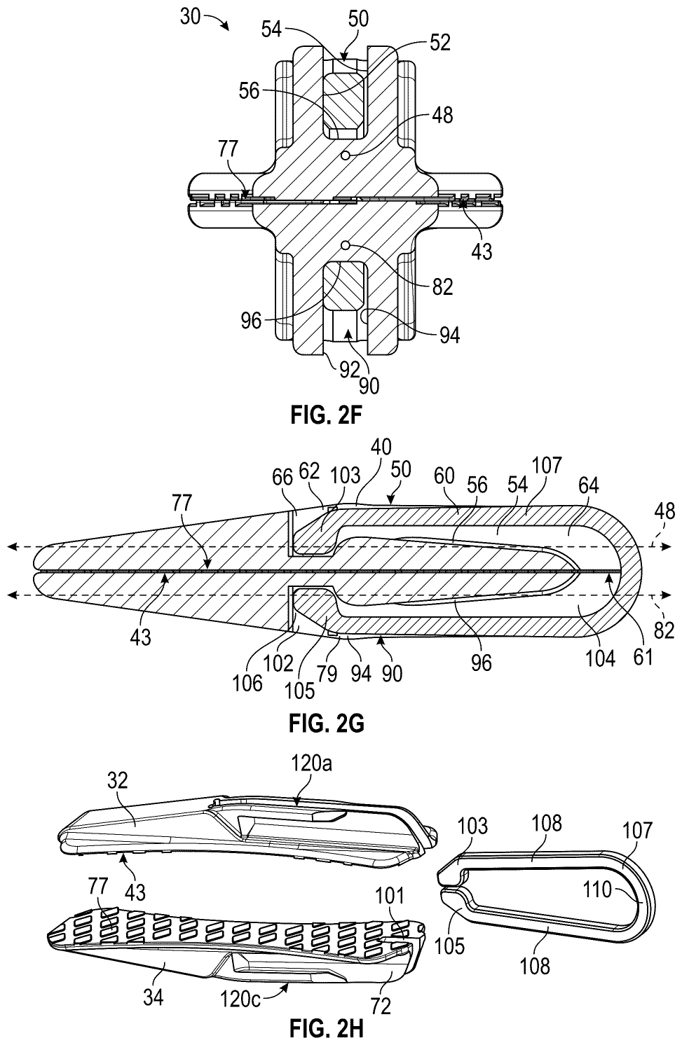

FIGS. 2A-2H illustrate an example of a clip 30 that may be utilized for a portion of a heart. The clip 30 may be configured to close a LAA 18, to reduce the possibility of clots or other undesired materials stemming from the LAA 18 from traveling into the bloodstream.

The clip 30 may include a first jaw 32 and a second jaw 34. The first jaw 32 may extend from a first end portion 36 to a second end portion 38 along a length of the first jaw 32. The first jaw 32 may have an elongate shape. The first jaw 32 may be configured as an elongate beam. The first jaw 32 may extend along a longitudinal axis 48 (marked in FIGS. 2F and 2G). The first jaw 32 may have a central portion 40 between the first end portion 36 and the second end portion 38.

The first end portion 36 may comprise a proximal end portion of the clip 30. The second end portion 38 may comprise a distal end portion of the clip 30.

The first jaw 32 may include an outer surface 42, a compression surface 43 (marked in FIGS. 2F and 2G), and two side surfaces 44, 46 (with side surface 46 shown in FIG. 2B) each extending from the compression surface 43 to the outer surface 42. The two side surfaces 44, 46 may face opposite each other. The compression surface 43 may face opposite the outer surface 42 and towards the second jaw 34.

The first jaw 32 may include a first end surface 47 positioned at the first end portion 36 of the first jaw 32. The first jaw 32 may include a second end surface 49 positioned at the second end portion 38 of the first jaw 32. The first end surface 47 may comprise a proximal end surface of the first jaw 32 and the second end surface 49 may comprise a distal end surface of the first jaw 32.

In examples, the second end portion 38 of the first jaw 32 may be tapered, such that a rounded tip 51 of the first jaw 32 is provided. The rounded tip 51 of the first jaw 32 may allow the first jaw 32 to be atraumatic to a patient's body upon insertion into the body and deployment to a desired location. In examples, the first end portion 36 of the first jaw 32 may be tapered.

In examples, one or more of the side surfaces 44, 46 may be curved. The curvature may have a variety of forms and may comprise a concave curvature relative to the first jaw 32 (as shown in FIG. 2D for example). Referring to FIG. 2D, for example, the side surface 44 being curved concave relative to the first jaw 32 is shown. The curvature may have a constant radius of curvature, or may have a varied radius of curvature in examples. The curvature may extend from the first end portion 36 of the clip 30 to the second end portion 38 of the clip 30 or may extend for a portion of the clip 30. In examples, the side surface 46 may be curved concave relative to the first jaw 32. The curvature may comprise a same curvature as the side surface 44 or may comprise a different curvature.

A curvature of one or more of the side surfaces 44, 46 may allow the clip 30 to contour to a shape of a portion of the heart upon deployment. For example, the surface 44 may contour to a shape of the wall of the left atrium upon deployment. The surface 46 may contour to the shape of the wall of the left atrium upon the clip 30 being deployed in an opposite orientation (in which the side surface 46 faces the wall of the left atrium). Other configurations of curvature may be utilized in examples.

The outer surface 42 of the first jaw 32 may include a channel 50. FIG. 2F illustrates a cross sectional view of the clip 30 along line 2F-2F in FIG. 2A, and FIG. 2G illustrates a cross sectional view of the clip 30 along line 2G-2G in FIG. 2A. Referring to FIGS. 2F and 2G, the channel 50 may include a first side wall 52, a second side wall 54, and a lower wall 56 of the channel 50. The upper portion of the channel 50 may remain open for a spring 60 to pass through. The channel 50 may extend from the central portion 40 of the first jaw 32 to the first end portion 36 of the first jaw 32. In examples, the channel 50 may include a cut out portion 61 of the compression surface 43 at the first end portion 36 to allow for the spring 60 to pass through the compression surface 43.

The channel 50 may include a first end 62 and a second end 64, with the second end 64 being opened to allow for the spring 60 to pass through.

A coupler 66 may be positioned at the first end 62 of the channel 50 and configured to receive an end of the spring 60. The coupler 66 may comprise a cavity in the first jaw 32 or may have another configuration as desired.

The second jaw 34 may extend from a first end portion 72 to a second end portion 74 along a length of the second jaw 34. The second jaw 34 may have an elongate shape. The second jaw 34 may be configured as an elongate beam. The second jaw 34 may extend along a longitudinal axis 82 (marked in FIGS. 2F and 2G). The second jaw 34 may have a central portion 79 between the first end portion 72 and the second end portion 74.

The first end portion 72 may comprise a proximal end portion of the clip 30. The second end portion 74 may comprise a distal end portion of the clip 30.

The second jaw 34 may include an outer surface 76 (marked in FIG. 2B), a compression surface 77 (marked in FIG. 2H), and two side surfaces 78, 80 (with side surface 80 shown in FIG. 2B) each extending from the compression surface 77 to the outer surface 76. The two side surfaces 78, 80 may face opposite each other. The compression surface 77 may face opposite the outer surface 76 and towards the first jaw 32.

The second jaw 34 may include a first end surface 81 positioned at the first end portion 72 of the second jaw 34. The second jaw 34 may include a second end surface 83 positioned at the second end portion 74 of the second jaw 34. The first end surface 81 may comprise a proximal end surface of the second jaw 34 and the second end surface 83 may comprise a distal end surface of the second jaw 34.

In examples, the second end portion 74 of the second jaw 34 may be tapered, such that a rounded tip 85 of the second jaw 34 is provided. The rounded tip 85 of the second jaw 34 may allow the second jaw 34 to be atraumatic to a patient's body upon insertion into the body and deployment to a desired location. In examples, the first end portion 72 of the second jaw 34 may be tapered.

In examples, one or more of the side surfaces 78, 80 may be curved. The one or more side surfaces 78, 80 may be curved in a similar manner as described regarding the curvature of the side surfaces 44, 46. The curvature of one or more of the side surfaces 78, 80 may be different from the curvature of the side surfaces 44, 46 in examples.

The outer surface 76 of the second jaw 34 may include a channel 90. Referring to FIGS. 2F and 2G, the channel 90 may include a first side wall 92, a second side wall 94, and a lower wall 96 of the channel 90. The upper portion of the channel 90 may remain open for the spring 60 to pass through. The channel 90 may extend from the central portion 79 of the second jaw 34 to the first end portion 72 of the second jaw 34. In examples, the channel 90 may include a cut out portion 101 (marked in FIG. 2H) of the compression surface 77 at the first end portion 72 to allow for the spring 60 to pass through the compression surface 77.

The channel 90 may include a first end 102 and a second end 104, with the second end 104 being opened to allow for the spring 60 to pass through.

A coupler 106 may be positioned at the first end 102 of the channel 90 and configured to receive an end of the spring 60. The coupler 106 may comprise a cavity in the second jaw 34 or may have another configuration as desired.

In examples, the compression surfaces 43, 77 of the respective jaws 32, 34 may be coated with a medical grade soft material to make the compression surfaces of the jaws 32, 34 atraumatic if desired.

Referring to FIG. 2A, the spring 60 may be configured to force the first jaw 32 and the second jaw 34 together to compress a portion of the heart with the first jaw 32 and the second jaw 34. The spring 60 may extend over the outer surface 42 of the first jaw 32 and the outer surface 76 of the second jaw 34 and may be configured to force the first jaw 32 and the second jaw 34 together to compress the portion of the heart between the compression surface 43 of the first jaw 32 (marked in FIG. 2G) and the compression surface 77 of the second jaw 34 (marked in FIG. 2H). The spring 60, for example may have a “C” shape with a first end 103 and a second end 105 and a loop 107 coupled to the first end 103 and the second end 105 (marked in FIG. 2H). The loop 107 may include straight portions 108 coupled to a curved portion 110 forming the curve of the “C” shape. The first end 103 of the spring 60 may be coupled to the central portion 40 of the first jaw 32 (marked in FIG. 2G) and the second end 105 of the spring 60 may be coupled to the central portion 79 of the second jaw 34 (marked in FIG. 2G), and the loop 107 may extend towards the first end portion 36 of the first jaw 32 and the first end portion 72 of the second jaw 34.

The loop 107 may extend within the channel 50 of the first jaw 32 and the channel 90 of the second jaw 34. The loop 107 may be positioned within the channel 50 of the first jaw 32 and the channel 90 of the second jaw 34 between the respective side walls 52, 54 of the channel 50 and the side walls 92, 94 of the channel 90 (marked in FIG. 2F). The loop 107 may be spaced from the respective lower walls 56, 96 of the channels 50, 90 such that the loop 107 may move towards the respective lower walls 56, 96 upon the clip 30 being in an opened state. The spring 60, for example, may pivot with respect to the lower walls 56, 96 upon the clip 30 being in an opened state.

The position of the loop 107 within the channels 50, 90 may allow for expansion of the clip 30. The position of the loop 107 within the channels 50, 90 may reduce the possibility of twisting of the spring 60 during opening or closing of the clip 30.

The loop 107 may reside within the channels 50, 90 continuously and may reduce the possibility of the jaws 32, 34 from moving distally and side to side.

The spring 60 may extend within the plane of movement of the first jaw 32 and the second jaw 34.

The spring 60 may be configured to allow the clip 30 to move from an opened state to a closed state, yet force the clip 30 towards the closed state. The spring 60 accordingly may provide a force that moves the compression surfaces 43, 77 of the first jaw 32 and second jaw 34 towards each other to compress a portion of the heart therein. The spring 60 may be configured to keep the jaws 32, 34 under positive compression at all times, even at rest. The spring 60 may have a “C” shape to allow the second end portions 38, 74 of the first jaw 32 and the second jaw 34 respectively to form an axial opening for a space between the first jaw 32 and the second jaw 34 for receiving the portion of the heart. The space may comprise a compression channel between the first jaw 32 and the second jaw 34. The axial opening may comprise an opening at the second end portions 38, 74 of the first jaw 32 and second jaw 34. The clip 30 may be positioned in an opened state and with the portion of the heart slid through the axial opening and into the space. In examples, other methods of entry into the space may be provided (e.g., along an axis of the LAA).

The second end portion 38 of the first jaw 32 and the second end portion 74 of the second jaw 34 may form an open end of the clip 30. The first end portion 36 of the first jaw 32 and the first end portion 72 of the second jaw 34 may form a closed end of the clip 30.

The loop 107 of the spring 60 may form a boundary of the space between the first jaw 32 and the second jaw 34. The compression channel may be closed at the first end portions 36, 72 of the first jaw 32 and second jaw 34. The loop 107 of the spring 60 may close the space at the first end portion 36 of the first jaw 32 and the first end portion 72 of the second jaw 34. The loop 107 accordingly may prevent the clip 30 from sliding distally with respect to the LAA 18 upon deployment, and may prevent the tissue of the LAA 18 from protruding further than the loop 107 upon compression of the LAA 18.

In examples, the clip 30 may include one or more elongate couplers 120a-d (marked in FIGS. 2A and 2B). The elongate couplers 120a-d may be positioned on one or more of the first jaw 32 or the second jaw 34 and may extend along a length of the respective first jaw 32 or second jaw 34. The elongate couplers 120a-d may extend along the longitudinal dimension of the respective first jaw 32 or second jaw 34. In examples, the elongate couplers 120a-d may extend parallel with a respective one of the longitudinal axis 48 of the first jaw 32 or the longitudinal axis 82 of the second jaw 34 (marked in FIG. 2G). The one or more elongate couplers 120a-d may be configured to engage an expansion apparatus for the clip 30.

In examples, the one or more elongate couplers 120a-d may include one or more protrusions 124a-d (marked in FIGS. 2A and 2B). The protrusion 124a may extend from the side surface 44 of the first jaw 32 and the protrusion 124b may extend from the side surface 46 of the first jaw 32 (marked in FIG. 2B). The protrusion 124c may extend from the side surface 78 of the second jaw 34 and the protrusion 124d may extend from the side surface 80 of the second jaw 34 (marked in FIG. 2B).

The protrusions 124a-d may extend along the length of the respective first jaw 32 or second jaw 34. The protrusions 124a-d may form rails extending along the length of the respective first jaw 32 or second jaw 34. An outer surface 126a of the protrusion 124a may comprise a portion of the outer surface 42 of the first jaw 32. An inner surface 128a of the protrusion 124a may face opposite the outer surface 126a and may face towards a channel 130a extending along the side surface 44 of the first jaw 32. The channel 130a may extend along the length of the first jaw 32. The protrusion 124b may be similarly configured on the side surface 46 of the first jaw 32. The protrusions 124c, d may be similarly configured on the respective side surfaces 78, 80 of the second jaw 34. In examples, the protrusions 124a-d may have similar configurations as each other or differing configurations.

The protrusions 124a-d may each extend proximally from the central portions 40, 79 of the respective first jaw 32 and second jaw 34 towards the first end portions 36, 72 of the respective first jaw 32 and second jaw 34. As shown in FIGS. 2A and 2B, the protrusions 124a-d may extend to a mid point between the central portions 40, 79 and the respective first end surfaces 47, 81. The protrusions 124a-d may each include a respective side surface 132a-d that may be angled or ramped at the mid point between the central portions 40, 79 and the respective first end surfaces 47, 81. The angled side surface 132a-d may allow for improved sliding engagement with the expansion apparatus.

In examples, the protrusions 124a-d may include a respective elongate side surface 134a-d (marked in FIGS. 2A and 2B) that may comprise a portion of the respective side surface 44, 46, 78, 80.

The configuration of the one or more elongate couplers 120a-d may vary in examples.

FIG. 2C illustrates a side view of the clip 30. FIG. 2D illustrates a top view of the clip 30. FIG. 2E illustrates a proximal end view of the clip 30.

FIG. 2H illustrates an exploded view of the clip 30. In examples, one or more of the compression surface 43 of the first jaw 32 or the compression surface 77 of the second jaw 34 may include a textured surface that may enhance grip with the portion of the heart being occluded. In examples, one or more of the compression surfaces 43, 77 may comprise smooth surfaces.

In examples, the one or more elongate couplers 120a-d may be configured to slidably engage with an expansion apparatus. The one or more elongate couplers 120a-d may be configured to engage the expansion apparatus advanced in a direction from the proximal end or closed end of the clip 30 towards the distal end or open end of the clip. For example, the one or more elongate couplers 120a-d may be configured such that the expansion apparatus is advanced from the first end portions 36, 72 of the jaws 32, 34 towards the second end portions 38, 74 of the jaws 32, 34.

Variations in the configuration of the clip 30 may be provided as desired. Features of the clip 30 may be utilized solely or in combination with any other example herein.

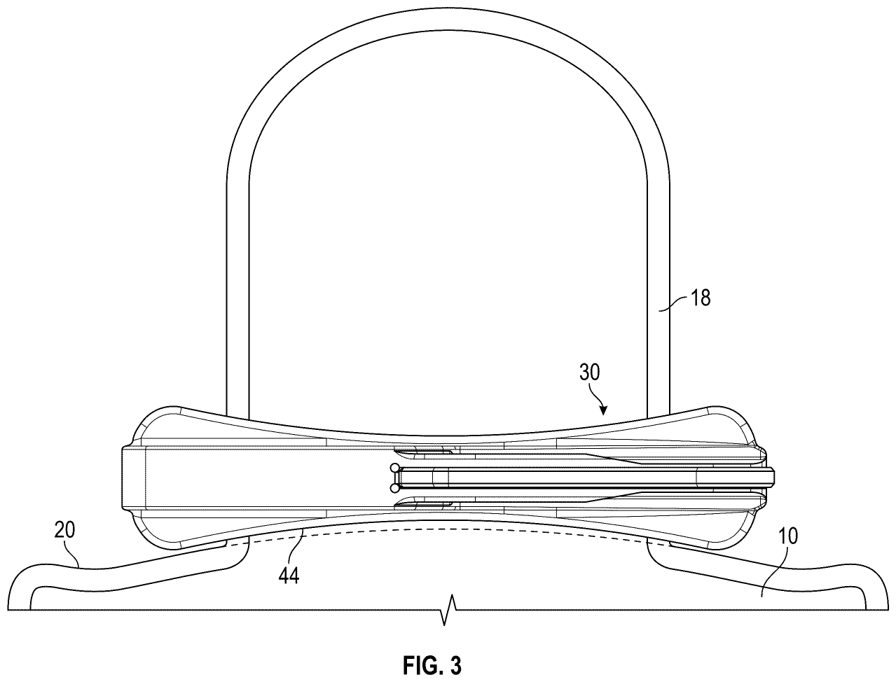

FIG. 3 illustrates a top view of the clip 30 upon deployment, occluding the LAA 18. The side surface 44 of the clip 30 may contour to a shape of the left atrium 10 in examples.

If desired, the clip 30 may be captured utilizing an expansion apparatus. The expansion apparatus, for example, may be slid onto the elongate couplers 120a-d in vivo. The clip 30 may be repositioned or entirely removed from the LAA 18 as desired. An expansion apparatus may be utilized for deployment or capture of the clip 30.

The clip 30 may be utilized to close the LAA 18, yet in examples other portions of a heart may be clipped or closed via use of the clip 30. In examples, other portions of a body, such as a tubular vessel or other portions of a body may be closed with the clip 30. Deployment may be via an expansion apparatus or via another method as desired.

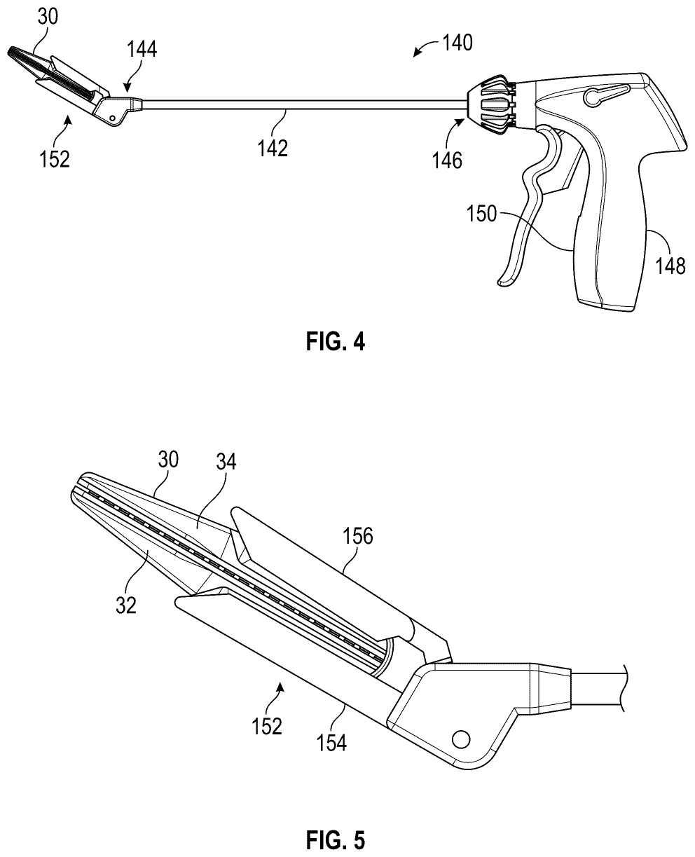

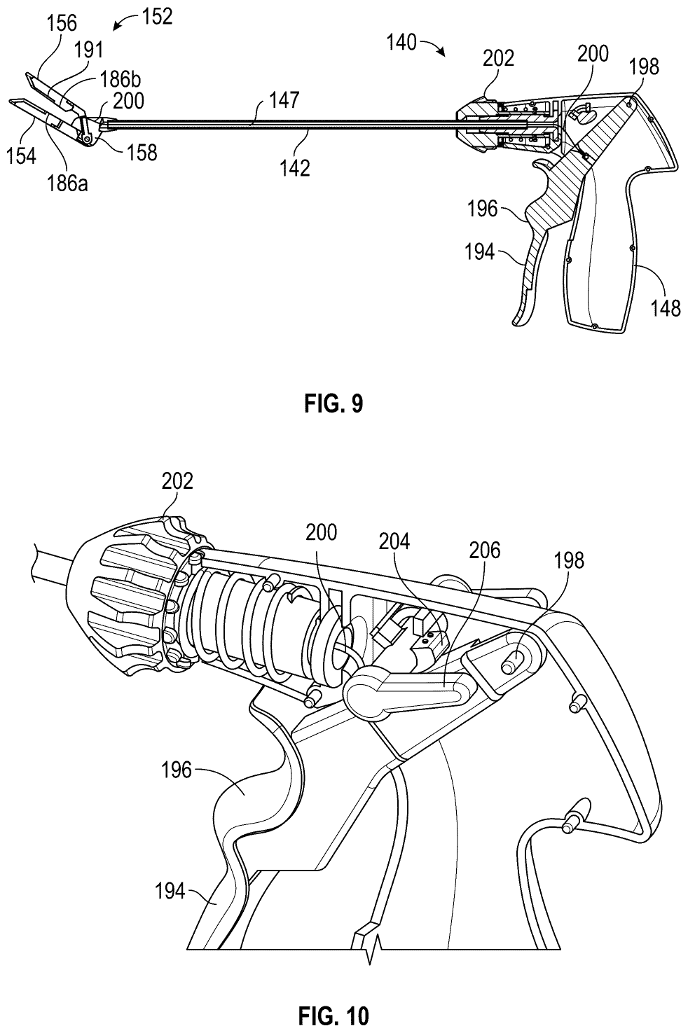

FIG. 4 illustrates a side view of an expansion apparatus 140 that may be utilized in examples. The expansion apparatus 140 may include an elongate shaft 142 having a first end portion 144 or distal end portion and a second end portion 146 or proximal end portion. The elongate shaft 142 may comprise a rigid shaft or may comprise a flexible shaft in examples. The elongate shaft 142 may be configured for one or more components of a control mechanism to pass therethrough, to extend to the engagement portion 152 in examples. The elongate shaft 142 may include an interior lumen 147 (marked in FIG. 9) for example.

The expansion apparatus 140 may include a handle 148 in examples. The handle 148 may be positioned at the second end portion 146 or proximal end portion of the elongate shaft 142. The handle 148 may include a grip portion 150 for a user to grip in examples.

The expansion apparatus 140 may include an engagement portion 152. The engagement portion 152 may be configured to engage the clip 30 in examples. The engagement portion 152 may be positioned at the first end portion 144 of the elongate shaft 142 in examples.

FIG. 5 illustrates a side view of the clip 30 engaged by the engagement portion 152 of the expansion apparatus 140.

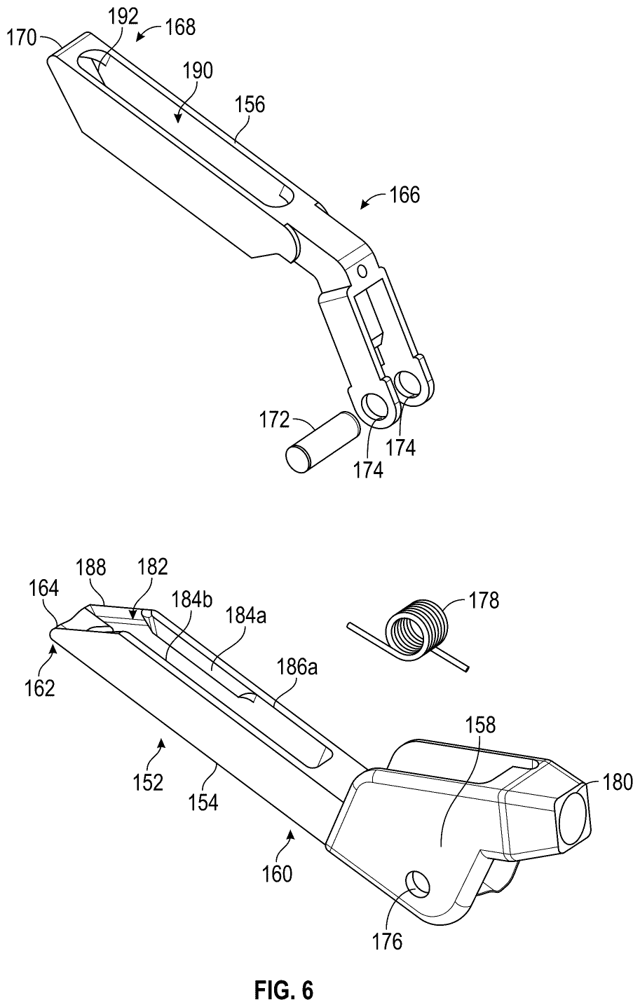

FIG. 6 illustrates an assembly view of the engagement portion 152. The engagement portion 152 may have at least two arms. The engagement portion 152 may have a first arm 154 and a second arm 156. The first arm 154 may be configured to engage the first jaw 32 of the clip 30, and the second arm 156 may be configured to engage the second jaw 34 of the clip 30. The first arm 154 may be configured to move relative to the second arm 156 to open or close the clip 30.

The arms 154, 156 may each extend from a base 158. The first arm 154 may have a proximal end portion 160 and a distal end portion 162. The first arm 154 may extend longitudinally and may be for sliding engagement with the first jaw 32 of the clip 30. The proximal end portion 160 may be fixedly coupled to the base 158 in examples. The first arm 154 may extend longitudinally from the proximal end portion 160 to the distal end portion 162. The distal end portion 162 may comprise a distal tip 164 of the first arm 154.

The second arm 156 may have a proximal end portion 166 and a distal end portion 168. The second arm 156 may extend longitudinally and may be for sliding engagement with the second jaw 34 of the clip 30. The proximal end portion 166 may be pivotally coupled to the base 158 in examples. The second arm 156 may extend longitudinally from the proximal end portion 166 to the distal end portion 168. The distal end portion 168 may comprise a distal tip 170 of the second arm 156.

The second arm 156 may couple to the base 158 through use of a pivot axle 172, although other forms of pivotal coupling may be utilized in examples. The pivot axle 172 may pass through openings 174 in the proximal end portion 166 of the second arm 156 and through openings 176 in the base 158, although other configurations may be utilized in examples. The second arm 156 may be configured to pivot at the base 158 to open or close the space between the arms 154, 156 and accordingly open or close the clip 30.

In examples, a spring 178 may be utilized that may bias the second arm 156. The spring 178, for example, may comprise a torsion spring or may have other forms in examples. The spring 178 may bias the second arm 156 in a closed configuration in examples. Other configurations may be utilized in examples (e.g., biased in an opened configuration). The bias of the spring 178 may be overcome with a force applied to the second arm 156 by a control mechanism (e.g., a tether of a control mechanism). Other configurations may be utilized in examples.

In examples, at least one of the first arm 154 or the second arm 156 may be pivotally coupled to the base 158. In examples, both the first arm 154 and the second arm 156 may be pivotally coupled to the base 158.

In examples, one of the first arm 154 or the second arm 156 may be fixedly coupled to the base 158. The fixed coupling to the base 158 may allow for a more stable approach to a delivery or implantation site. For example, the number of movable components may be reduced through use of one of the first arm 154 or the second arm 156 being fixedly coupled to the base 158. The engagement portion 152 may be more compact than an example in which both arms 154, 156 are pivotal. A user may further have an improved datum point for a deployment procedure, as the user may be aware that only one arm will pivot, and can determine position off of a stable fixed arm. A single one of the first arm 154 or the second arm 156 may be pivotally coupled to the base 158 to allow for the arms 154, 156 to move relative to each other to open or close the space between the arms 154, 156 and accordingly open or close the clip 30.

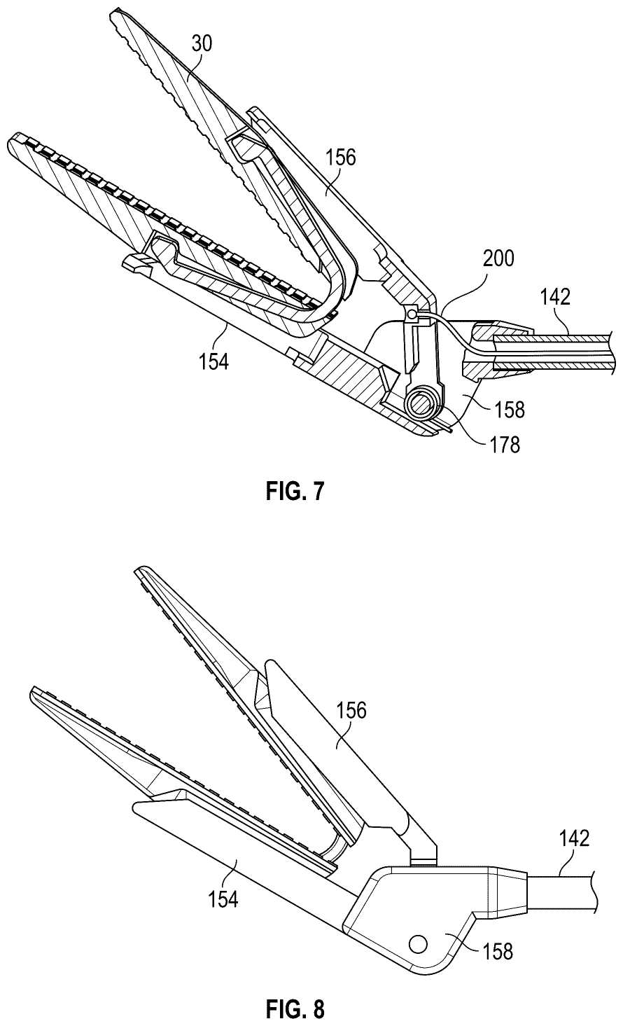

The arms 154, 156 may be configured to move towards each other to close the clip 30 and move away from each other to open the clip 30. The angle between the arms 154, 156 may vary in the opened configuration and the closed configuration. For example, in a closed configuration (as represented in FIG. 5), the arms 154, 156 may extend parallel with each other, or otherwise have a reduced or acute angle between them. In an opened configuration (as represented in FIGS. 7 and 8), the arms 154, 156 may have a greater angle relative to each other than in the closed configuration.

Referring to FIG. 6, the base 158 in examples may include a coupler 180 for coupling with the elongate shaft 142. The coupler 180, for example, may comprise an aperture for the first end portion 144 of the elongate shaft 142 to insert into. The base 158 may couple to the elongate shaft 142 with a pivot coupling, or may be fixed in relation to the elongate shaft 142.

The first arm 154 may include a channel 182 for engaging the first jaw 32 of the clip 30. The channel 182 may receive at least a portion of the first jaw 32. The channel 182 may extend longitudinally along a length of the first arm 154. One or more rails 184a, b may extend along the channel 182 and may be configured to retain the first jaw 32 within the channel 182. The rails 184a, b may be for sliding engagement with the first jaw 32. The rails 184a, b may comprise opposed rails on opposite sides of the channel 182. The rails 184a, b may bound an inner opening 186a of the first arm 154 that faces towards the second arm 156. The first arm 154 may include a distal tip 164 that may include an opening 188 for the channel 182. The opening 188 may be for sliding release of the clip 30 from the first arm 154.

The second arm 156 may be configured similarly as the first arm 154. For example, the second arm 156 may include a channel 190 for engaging the second jaw 34 of the clip 30. The channel 190 may extend along a length of the second jaw 34. The channel 190 may receive at least a portion of the second jaw 34. One or more rails 191 (marked in FIG. 9) may extend along the channel 190 and may be configured to retain the second jaw 34 within the channel 190. The rails 191 may be for sliding engagement with the second jaw 34. The rails 191 may comprise opposed rails on opposite sides of the channel 190, configured similarly as the rails 184a, b of the first jaw 32. The rails 191 may bound an inner opening 186b (marked in FIG. 9) of the second arm 156 that faces towards the first arm 154, in a similar manner as the inner opening 186a. The second arm 156 may include a distal tip 170 that may include an opening 192 for the channel 190. The opening 192 may be for sliding release of the clip 30 from the second arm 156.

The first arm 154 and second arm 156 may be configured to be positioned in a plane of movement of the first arm 154 and the second arm 156, with elongate inner openings 186a, b of the channels 182, 190 facing towards each other.

The first arm 154 may be configured for the first jaw 32 to be slid proximally in a direction from the distal tip 164 towards the proximal end portion 160 of the first arm 154. The second arm 156 may be configured for the second jaw 34 to be slid proximally in a direction from the distal tip 170 of the second arm 156 towards the proximal end portion 166 of the second arm 156. The rails 184a, b of the first arm 154 may slidingly engage the elongate couplers 120a, b of the first jaw 32. The rails 191 of the second arm 156 may engage the elongate couplers 120c, d of the second jaw 34. The rails 184a, b, for example, may overlap the inner surfaces of the elongate couplers 120a, b of the first jaw 32 and the rails 191 may overlap the inner surfaces of the elongate couplers 120c, d. The elongate couplers 120a-d may be inserted through the openings 188, 192 of the respective arms 154, 156.

The first arm 154 may be configured to be moved away from the second arm 156 to open the clip 30. An opened configuration is shown in FIGS. 7 and 8 for example. The first arm 154 may be configured to be moved towards the second arm 156 to close the clip 30.

In examples, a control mechanism may be utilized for controlling opening and closing of the first arm 154 relative to the second arm 156. The control mechanism may have a variety of forms in examples. FIG. 9, for example, illustrates a cross sectional view of the expansion apparatus 140. The control mechanism may include a control device 194. The control device 194 may be positioned upon the handle 148 in examples, or may be alternatively positioned in examples. The control device 194 may comprise a control lever arm 196 in examples. The control lever arm 196 may include a trigger or grip portion of the control device 194 in examples, or may have other configurations. The control lever arm 196 may extend into an interior of the handle 148 as shown in FIG. 9, or other configurations may be utilized in examples.

The control lever arm 196 may be configured to pivot, and may be pivotally coupled to the handle 148 at a pivot 198. The control lever arm 196 may rotate about the pivot 198. In examples, other configurations of control devices 194 may be utilized.

The control mechanism may include a tether 200 in examples. The tether 200 may extend from the control device 194 to the engagement portion 152 of the expansion apparatus 140 in examples. Other configurations may be utilized in examples. The tether 200 may have a proximal end portion that may couple to the control device 194 and may have a distal end portion that may couple to the second arm 156. The tether 200 may extend within the interior lumen 147 of the elongate shaft 142 in examples. Other configurations may be utilized in examples.

A retraction or other retracting movement of the control device 194 may provide a tension or retracting movement upon the tether 200. The tether 200 may retract and accordingly provide a tension or retraction force against the second arm 156. The pivotal coupling of the second arm 156 to the base 158 may allow the second arm 156 to pivot and move to the opened configuration. Upon release of the control device 194, the force applied by the spring 178 (marked in FIG. 7) may cause the second arm 156 to return to the closed configuration. As such, selective opening or closing of the arms 154, 156 may result.

In examples, a rotation mechanism may be utilized for controlling rotation of the engagement portion 152 about a longitudinal axis of the elongate shaft 142. The rotation mechanism, for example, may include a control device 202 in the form of a rotatable knob that may be utilized to rotate the engagement portion 152 about the longitudinal axis of the elongate shaft 142. The control device 202, for example, may be fixedly coupled to the elongate shaft 142 such that rotation of the control device 202 produces rotation of the elongate shaft 142 and accordingly rotates the engagement portion 152. In such a configuration, the engagement portion 152 may be fixedly coupled to the elongate shaft 142. In other configurations, the control device 202 may have a coupling that extends to the engagement portion 152 and causes the engagement portion 152 to pivot relative to the elongate shaft 142. In such a configuration the engagement portion 152 may be pivotally coupled to the elongate shaft 142. Other configurations may be utilized.

In examples, a retention mechanism may be utilized that may be configured to retain the clip 30 to the engagement portion 152 to impede sliding release of the clip 30 from the first arm 154 and the second arm 156. The retention mechanism may have a variety of forms in examples. The retention mechanism may be utilized to secure the clip 30 to the engagement portion 152 such that the clip 30 does not release from the engagement portion 152 until desired. For example, upon approach to an implantation site or upon assembly or handling of the expansion apparatus 140, the clip 30 may be prevented from prematurely releasing from the engagement portion 152 by undesirably sliding longitudinally off of the engagement portion 152. A reduced possibility of unintended movement or force dislodging the clip 30 from the engagement portion 152 may result. The retention mechanism may be utilized to allow a user to release the clip 30 from the engagement portion 152 at a desired time. For example, a user may place the clip 30 in position at a desired implantation site, and then actuate the retention mechanism to release the clip 30 upon a final position being determined. As such, a premature release of the clip 30 may be reduced. In examples in which the expansion apparatus 140 is utilized for recapture or repositioning of the clip 30, the retention mechanism may be utilized to confirm securement of the clip 30 to the engagement portion 152.

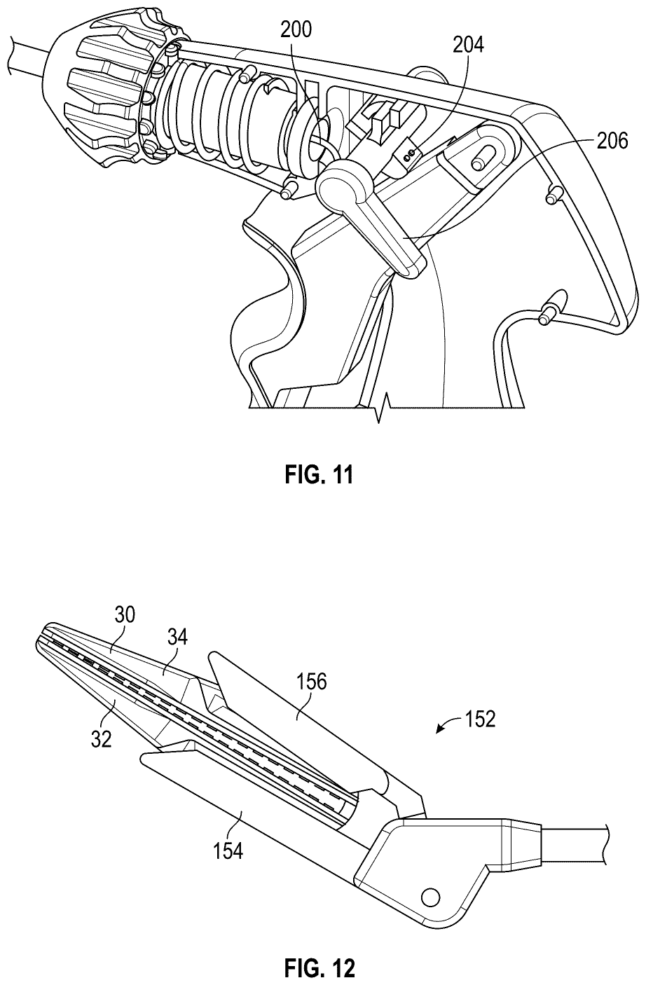

FIG. 10 illustrates an example of a retention mechanism that may be utilized in examples herein. The retention mechanism may include a cam body 204 in examples. The cam body 204 may be configured to apply a force to at least a portion of the control mechanism. The cam body 204, for example, may be rotatable from a configuration in which the cam body 204 does not apply the force to the portion of the control mechanism (shown in FIG. 10) to a configuration in which the cam body 204 applies the force to the portion of the control mechanism (shown in FIG. 11). The configuration shown in FIG. 10 may comprise a release configuration and the configuration shown in FIG. 11 may comprise a retention configuration.

The retention mechanism may include a control device 206 that may be operable by a user to selectively actuate the retention mechanism. The control device 206 as shown in FIG. 10 may comprise a lever, although other configurations may be utilized (e.g., a slider, a knob, among others). One or more of a lever, a slider, or a knob may be utilized as desired. The control device 206 may be operated by the user to actuate the cam body 204 between the release configuration and the retention configuration as desired.

The cam body 204 may be configured to apply a force to at least a portion of the control lever arm 196 in examples. In the retention configuration shown in FIG. 11, the cam body 204 has been rotated from the configuration shown in FIG. 10 such that a retraction of the control lever arm 196 results. The greater diameter of the portion of the cam body 204 contacting the control lever arm 196 in FIG. 11 produces the retraction of the control lever arm 196. The retraction of the control lever arm 196 may apply a tension or retraction force to the tether 200, which may correspondingly pivot the second arm 156.

FIG. 12 illustrates the configuration of the engagement portion 152 with the retention mechanism in the retention configuration indicated in FIG. 11. The second arm 156 has moved to an angle relative to the first arm 154 that is greater than the angle between the arms 156, 154 in the closed configuration shown in FIG. 5.

The retention mechanism accordingly provides a force from the first arm 154 to the first jaw 32 of the clip 30 in the configuration shown in FIG. 12. The force impedes sliding release of the clip 30 from the first arm 154 and the second arm 156. The force is an expansion force, due to the force of expansion applied by the arms 154, 156 to the respective jaws 32, 34. The force comprises a friction force that is caused by the friction between the rails 184a, b, 191 of the arms 154, 156 and the elongate couplers 120a, b, c, d of the first jaw 32 and second jaw 34. The friction may impede the sliding release of the clip 30 from the rails 184a, b, 191 of the arms 154, 156.

In examples, an interference fit between the rails 184a, b, 191 of the arms 154, 156 and the elongate couplers 120a, b, c, d of the first jaw 32 and second jaw 34 may result. The rails 184a, b, 191, for example, may be angled non-parallel with the elongate couplers 120a, b, c, d, to produce the interference fit. The interference fit may impede the sliding release of the clip 30 from the rails 184a, b, 191 of the arms 154, 156.

In a configuration as shown in FIG. 12, the first arm 154 and the second arm 156 may be held in an at least partially opened configuration to retain the clip 30 to the engagement portion 152. The arms 154, 156 may be held in such a configuration based on the retractive force applied to the tether 200 due to the cam body 204 pressing the control lever arm 196 proximally or in a retracted position. The amount of the retraction may be determined by the diameter of the cam body 204 utilized to press against the control lever arm 196 in a configuration shown in FIG. 11. The angle between the arms 154, 156 shown in FIG. 12 may be greater than the angle between the arms 154, 156 in the closed configuration shown in FIG. 5. The retention mechanism may provide a preload force upon the clip 30 to retain the clip 30 to the engagement portion 152.

In a method of operation, the clip 30 may be engaged with the engagement portion 152 with the arms 154, 156 held in the closed configuration shown in FIG. 5. The retention mechanism may be in the release configuration shown in FIG. 10. The retention mechanism may then be selectively actuated to the retention configuration represented in FIG. 11. The control device 206, for example, may be actuated to cause the cam body 204 to retract the control lever arm 196. The control lever arm 196 may be retracted by a distance that does not fully move the arms 154, 156 to the opened configuration. The clip 30 may be retained to the engagement portion 152 with the arms 154, 156 in a partially opened configuration as shown in FIG. 12.

In examples, the clip 30 may be advanced to the implantation site in the configuration shown in FIG. 12. The clip 30 may be secured from premature release from the engagement portion 152. The retention mechanism may retain the clip 30 to the engagement portion 152 to impede the sliding release of the clip 30 from the arms 154, 156. The control device 194 may be fully retracted at a desired time to move the arms 154, 156 to the opened configuration shown in FIGS. 7 and 8. The clip 30 may open to allow for placement at the desired implantation site (e.g., occluding the LAA). The control device 194 may then be released to move the arms 154, 156 back to the partially opened configuration shown in FIG. 12, yet with the portion of the occluded body between the jaws 32, 34. The retention mechanism may then be released or moved to the release configuration (as represented in FIG. 10) to allow the clip 30 to slidably release from the arms 154, 156. The clip 30 may be placed in a position as represented in FIG. 3 for example.

In examples, the method may be varied as desired. For example, the order of the steps may be varied. In examples, the retention mechanism may be actuated to the release configuration at or prior to the arms 154, 156 being in the opened configuration as represented in FIG. 7 or 8. Other modifications of the method may be provided as desired.

In examples, the retention mechanism may be utilized in a recapture or repositioning procedure for the clip 30. For example, the clip 30 may be deployed previously or a deployment procedure may be occurring. The arms 154, 156 may reengage the clip 30 by sliding onto the clip 30. The arms 154, 156 may be in a closed configuration as represented in FIG. 5. The retention mechanism may then be actuated to the retention configuration. The arms 154, 156 may then be placed in the opened configuration and the clip 30 may be repositioned or removed as desired. The retention mechanism may retain the clip 30 to the engagement portion 152 to impede sliding release of the clip 30 from the arms 154, 156. The retention mechanism may be actuated to the release configuration to allow a user to remove the clip 30 from the engagement portion 152 or to reposition the clip 30 as desired at an implantation site. The method may be modified as desired (e.g., the retention mechanism may be actuated to the retention configuration following the arms 154, 156 being moved to the opened configuration).

The features of FIGS. 4-12 may be utilized solely or in combination with any other example herein.

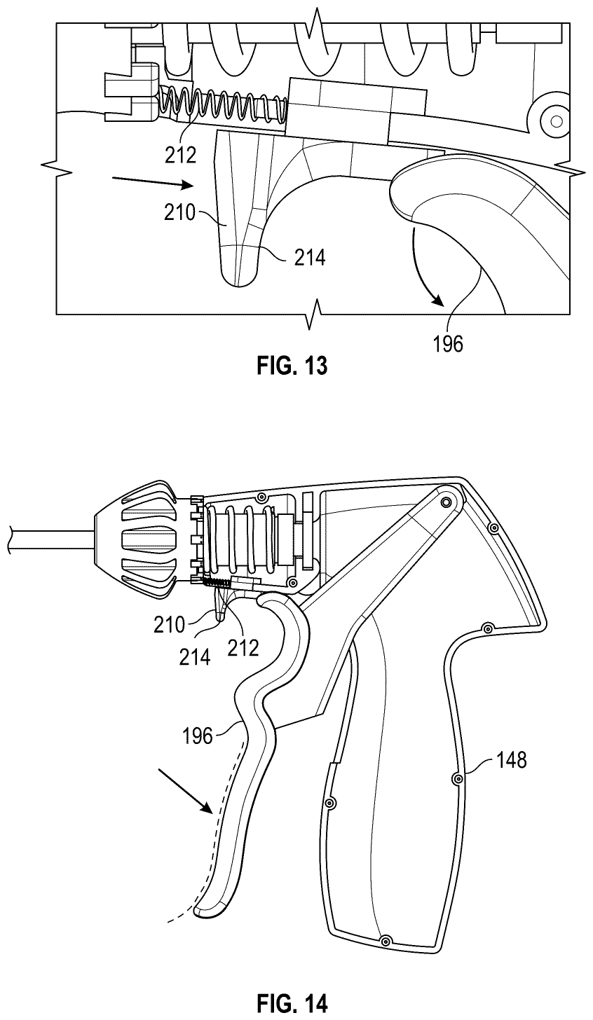

Various other forms of retention mechanisms may be utilized in examples. FIGS. 13-16, for example, illustrate an example in which the retention mechanism includes a slide body 210. The slide body 210 may be configured to apply a force (e.g., a normal force) to the control lever arm 196. The force may impede the movement of the control lever arm 196 to bias the control lever arm 196 in a partially retracted position, similar to the configuration represented in FIG. 11.

FIGS. 13 and 14 represent the retention mechanism in the retention configuration. The slide body 210 has been slid proximally towards the control lever arm 196 to impede the control lever arm 196 from moving to the closed position. The slide body 210 accordingly may comprise a spacer that spaces the position of the control lever arm 196 in a partially retracted position. The slide body 210 may be positioned on an outer surface of the handle 148 and between the control lever arm 196 and the outer surface of the handle 148.

In examples, a spring 212 may bias the slide body 210 to the proximal position (or the retention configuration). The bias of the spring 212 may be overcome with a user sliding the slide body 210 distally (or to the release configuration). A control device 214 (e.g., a slider) may be operated by a user to overcome the bias of the spring 212.

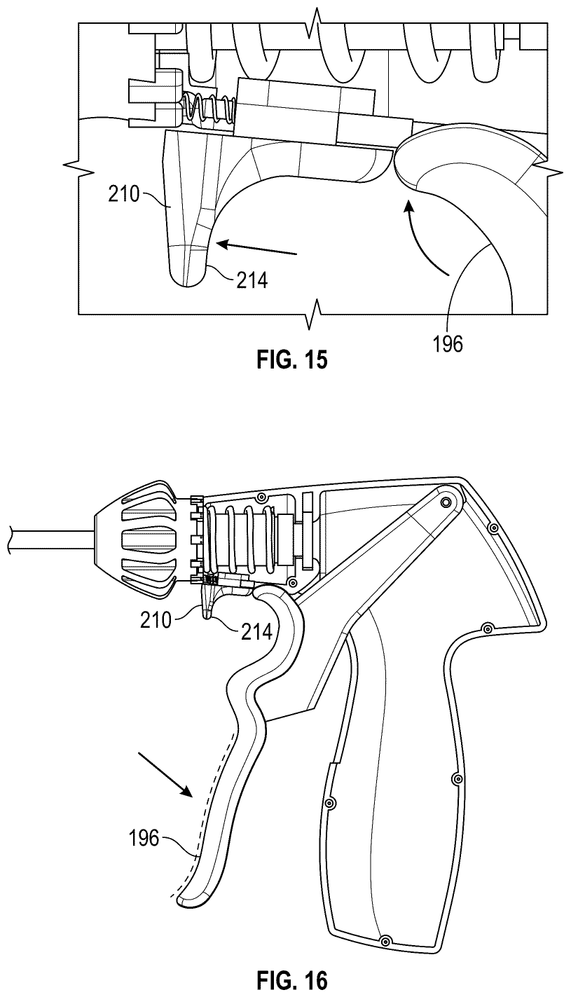

FIGS. 15 and 16, for example, represent the retention mechanism in the release configuration. The slide body 210 has been slid distally away from the control lever arm 196. As such, the control lever arm 196 may be moved distally to the closed position. The clip 30 may be released from the arms 154, 156. The retention mechanism may otherwise operate in a similar manner as discussed regarding the examples discussed in regard to FIGS. 4-12.

The features of FIGS. 13-16 may be utilized solely or in combination with any other example herein.

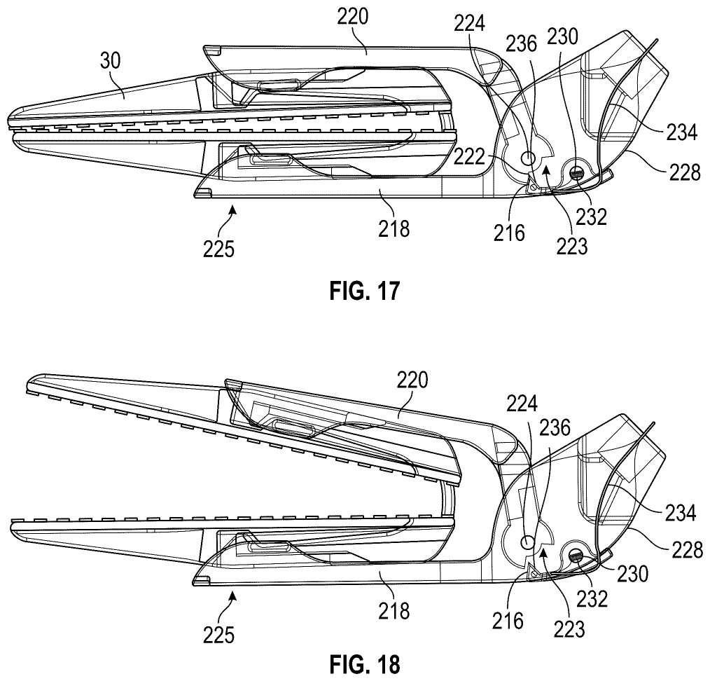

Various other forms of retention mechanisms may be utilized in examples. FIGS. 17-20, for example, illustrate an example in which the retention mechanism includes a stopper 216 that is configured to impede pivotal movement of the first arm 218 or the second arm 220. The first arm 218 and the second arm 220 may be configured similarly as the respective first arm 154 and second arm 156 unless stated otherwise. Features of the expansion apparatus 140 may be utilized with the examples of FIGS. 17-20 unless stated otherwise.

The stopper 216 may contact the proximal end portion of the second arm 220 to impede the second arm 220 from rotating to a closed configuration (as represented in FIG. 5 for example). The stopper 216, for example, may abut a contact surface 222 of the proximal end portion of the second arm 220. The contact surface 222 may bound an arc shaped cut out 223 in the proximal end portion of the second arm 220 that the stopper 216 may be positioned within. The arc shaped cut out 223 may extend about a pivot 224 for the second arm 220. The stopper 216 may contact the contact surface 222 to impede rotation of the second arm 220 to the closed configuration. The stopper 216 may comprise a wedge that sits within the clevis of the engagement portion and impedes movement of the second arm 220. The stopper 216 may impede full closure of the arms 218, 220 yet may allow for opening of the arms 218, 220 as desired.

The shape of the arc shaped cut out 223 may allow the second arm 220 to be rotated to the opened configuration as represented in FIG. 18. As such, the control mechanism may be allowed to move the second arm 220 to the opened configuration without interference from the stopper 216. Upon release of the control mechanism however, the stopper 216 may contact the contact surface 222, as represented in FIG. 17, to maintain the arms 218, 220 in a partially opened configuration, and retain the clip 30 to the engagement portion 225 in a similar manner as discussed in regard to FIGS. 4-16.

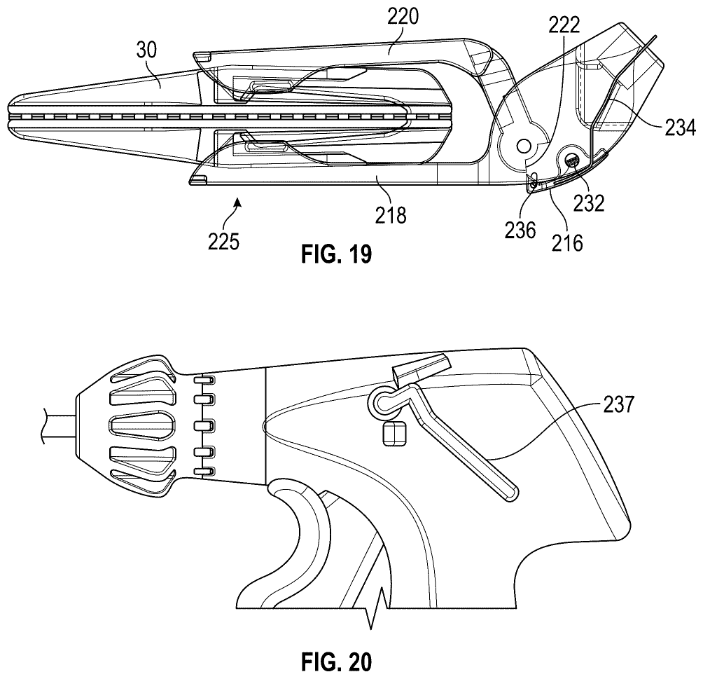

The stopper 216 may be configured to be moved to allow the retention mechanism to move to the release configuration. The stopper 216, for example, may be pivotally coupled to the base 228 with a pivot 230. A spring 232 may be provided that may bias the stopper 216 to the retention configuration (as represented in FIG. 17). A tether 234 may be coupled to the stopper 216, with a proximal force upon the tether 234 rotating the stopper 216 and moving the stopper 216 to the release configuration as represented in FIG. 19.

The stopper 216 in the release configuration may withdraw from contact surface 222 and allow the second arm 220 to rotate to the closed configuration (as represented in FIG. 19). As such, the clip 30 may slidably release from the arms 218, 220 in a similar manner as discussed in regard to FIGS. 4-16. The tether 234 may be released to allow the stopper 216 to return to the retention configuration as represented in FIG. 17. The spring 232 may bias the stopper 216 to return to the retention configuration.

In examples, the stopper 216 may include an angled surface 236 or tapered surface that may allow the stopper 216 to automatically return to the retention configuration upon the second arm 220 being moved towards the opened configuration from the closed configuration. The angled surface 236 may allow the stopper 216 to slide back into contact with the contact surface 222.

Referring to FIG. 20, in examples, a control device 237 may be utilized to selectively actuate the retention mechanism. The control device 237 may comprise a lever as shown in FIG. 20, or may have other forms as desired (e.g., a slider, or knob, or other form of control device). The control device 237 may be coupled to the tether 234 to selectively retract or tension the tether 234 to selectively actuate the stopper 216.

The retention mechanism may otherwise operate in a similar manner as discussed regarding the examples discussed in regard to FIGS. 4-16.

The features of FIGS. 17-20 may be utilized solely or in combination with any other example herein.

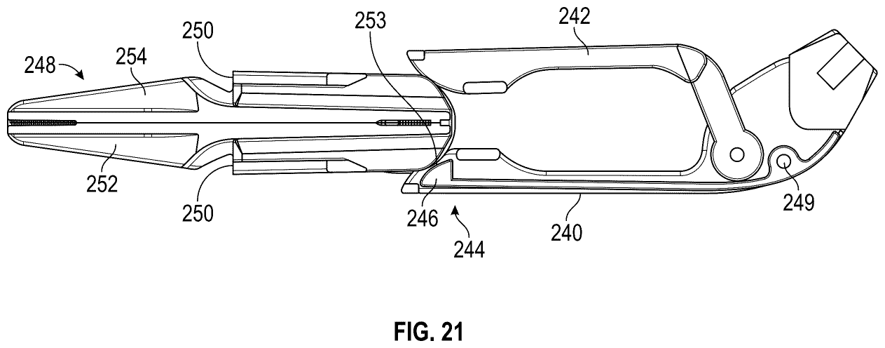

Various other forms of retention mechanisms may be utilized in examples. FIGS. 21-23 illustrate an example in which the retention mechanism provides a force from one or more of the arms 240, 242 to respective jaws of the clip to retain the clip to the engagement portion 244 to impede sliding release of the clip from the arms 240, 242. The first arm 240 and second arm 242 may be configured similarly as the respective first arm 154 and second arm 156 unless stated otherwise. Features of the expansion apparatus 140 may be utilized with the examples of FIGS. 21-23 unless stated otherwise.

The retention mechanism may include at least one protrusion 246 configured to engage at least a portion of the clip 248 to impede sliding release of the clip 248 from the arms 240, 242. A protrusion 246 as shown in FIG. 21, for example, may comprise a hook. The hook may be configured to enter into a recess 250 on the clip 248. One or more of the recesses 250 may be on the outer surface of the respective jaws 252, 254 of the clip 248 and may be shaped to receive the hook. The clip 248 may otherwise be configured similarly as the clip 30. The hook may create a mechanical interference with the clip 248 upon engaging the clip 248.

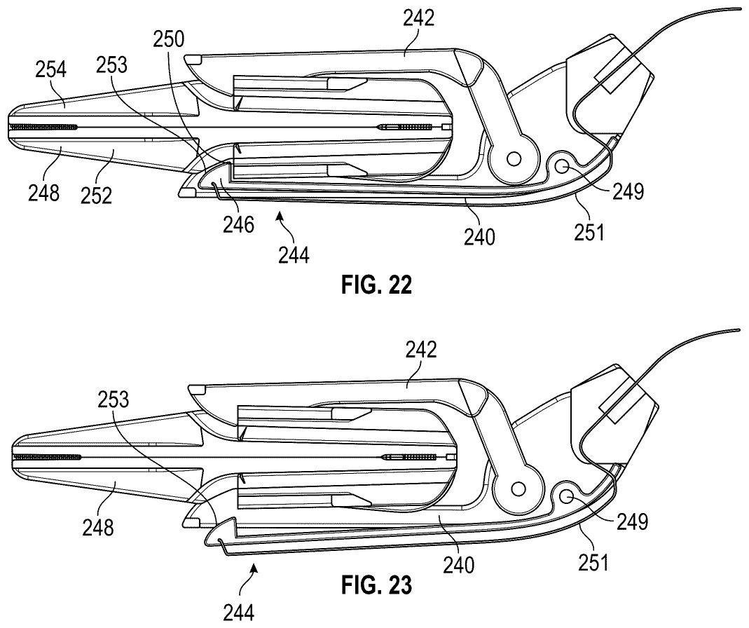

In examples, the protrusion 246 may be spring biased against at least a portion of the clip 248. The bias may be overcome via a force applied to the protrusion 246, for example, a tether 251 (marked in FIGS. 22 and 23) may be retracted to overcome the bias and retract the protrusion 246 as desired. The tether 251 may couple to a control device that may be configured similarly as other forms of control devices disclosed herein.

The protrusion 246 may be pivotally coupled to the engagement portion 244 at a pivot 249. The protrusion 246 may comprise a portion of a lever arm configured to pivot about the pivot 249 upon a retraction force being applied by the tether 251. The lever arm may extend along a length of the first arm 240, although other configurations may be utilized as desired.

FIG. 21 illustrates a configuration of the clip 248 relative to the engagement portion 244 prior to the clip 248 engaging the engagement portion 244. The protrusion 246 may have an angled surface or tapered surface 253 to allow the protrusion 246 to automatically retract from the clip 248 upon the clip 248 being inserted between the arms 240, 242 and slidably engaged with the arms 240, 242.

FIG. 22 illustrates the clip 248 in sliding engagement with the arms 240, 242. The protrusion 246 may insert into the recess 250. The protrusion 246 may engage the jaw 252 of the clip 248 to impede sliding release of the clip 248 from the arms 240, 242.

The protrusion 246 may be selectively actuatable from the retention configuration shown in FIG. 22 to the release configuration in which the protrusion 246 allows for sliding release of the clip 248 from the arms 240, 242. FIG. 23, for example, illustrates the release configuration in which the tether 251 has been retracted to retract the protrusion 246 from the clip 248. The clip 248 accordingly may slidingly release from the arms 240, 242.

The features of FIGS. 21-23 may be utilized solely or in combination with any other example herein.

Other forms of protrusions may be utilized in examples.

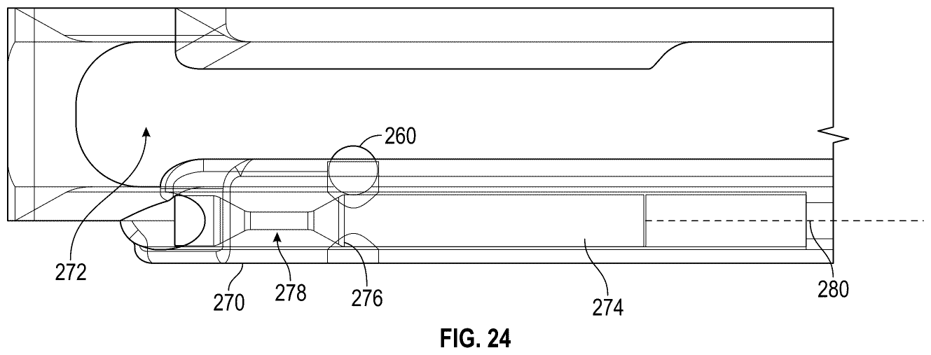

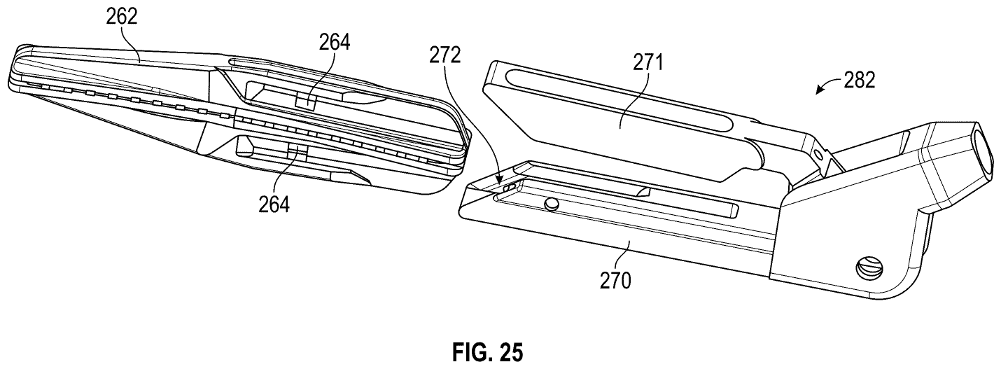

FIGS. 24-26, for example, illustrate a variation of the examples of FIGS. 21-23 in which the protrusion comprises a pin 260. The pin 260 may be configured to engage a clip 262 (marked in FIG. 25) by providing a friction force to the outer surface of the jaws of the clip 262 or by inserting into recesses 264 of the clip 262. The clip 262 may otherwise be configured similarly as the clip 30. The pin 260 may comprise a ball or may have another configuration in examples.

FIG. 24 illustrates a top view of the pin 260 in a retention configuration and protruding inward from the first arm 270. The pin 260 may protrude inward toward the channel 272 of the first arm 270. The pin 260 may be pressed towards the channel 272 with a biasing body 274 that may have a thicker diameter at a pressing portion 276 of the biasing body 274 relative to a release portion 278 of the biasing body 274. The biasing body 274 may comprise a shaft extending along the length of the first arm 270 although other configurations may be utilized in examples. The biasing body 274 may be spring biased towards the retention configuration shown in FIG. 24 for example. A tether 280 may be coupled to the biasing body 274. The tether 280 may be configured to be retracted in a similar manner as the tether 251 to cause the biasing body 274 to retract and shift the position of the pin 260 to the narrower release portion 278 of the biasing body 274. As such, the pin 260 may be actuated to a release configuration and may retract into the first arm 270.

FIG. 25 illustrates a position of the clip 262 relative to the engagement portion 282 prior to engagement between the clip 262 and the engagement portion 282. Features of the expansion apparatus 140 may be utilized with the examples of FIGS. 24-26 unless stated otherwise.

The clip 262 may be slidably engaged with the engagement portion 282 according to methods disclosed herein. The pin 260 may insert into one of the recesses 264 to impede sliding release of the clip 262 from the arms 270, 271.

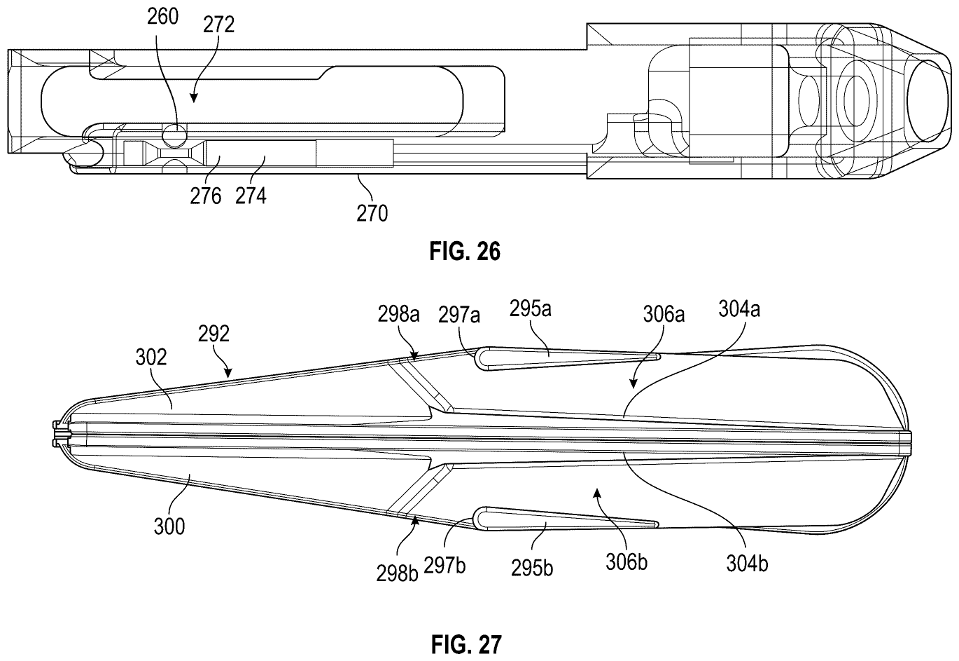

In examples, at a desired time, the pin 260 may be selectively actuated from the retention configuration (as represented in FIG. 24) to the release configuration (as represented in FIG. 26). The pin 260 may retract from the clip 262 due to a retraction of the biasing body 274 (as represented in FIG. 26). The clip 262 accordingly may slidingly release from the arms 270, 271.

The features of FIGS. 24-26 may be utilized solely or in combination with any other example herein.

Various other forms of retention mechanisms may be utilized in examples.

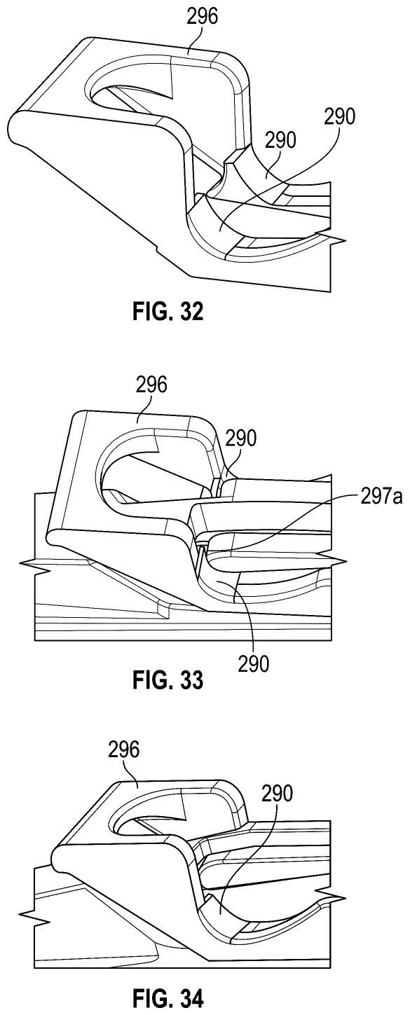

FIGS. 27-35, for example, illustrate a configuration in which the retention mechanism includes a barrier 290 (marked FIG. 32) that is configured to impede sliding release of the clip 292 from the arms 294, 296 (marked in FIG. 29).

FIG. 27 illustrates a side view of a clip 292 that may be utilized in examples. The clip 292 may include one or more tapered elongate couplers 295a, b for engaging rails of the respective arms 294, 296. The taper of the elongate couplers 295a, b may be wider towards the distal end of the clip 292 relative to the proximal end of the clip 292. A distal edge of the respective elongate couplers 295a, b may comprise contact surfaces 297a, b that may be configured to contact the respective barrier 290 of the arms 294, 296. A respective space 298a, b may be positioned distal of the contact surfaces 297a, b for receiving the respective barrier 290.

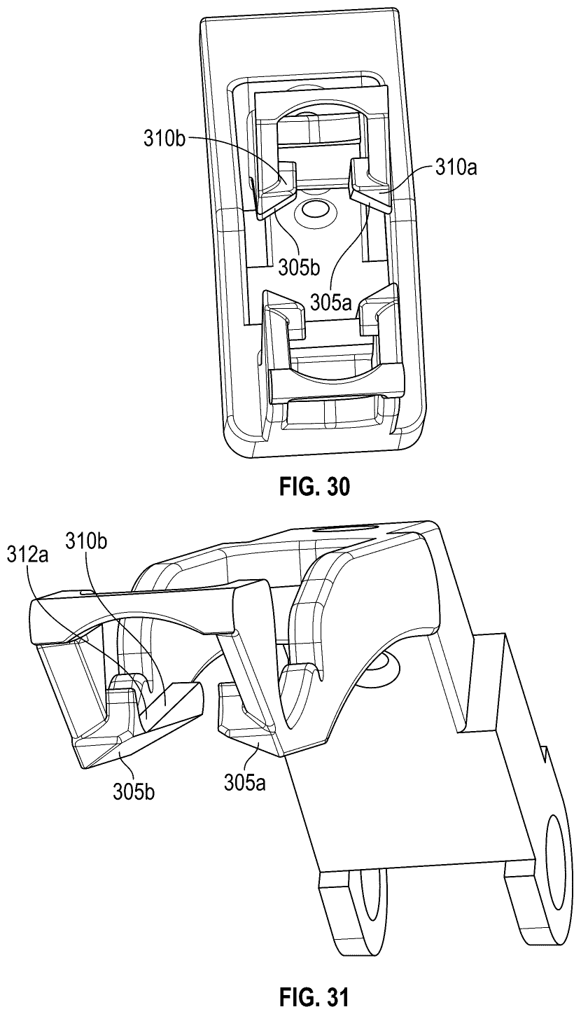

In examples, the jaws 300, 302 of the clip 292 may include angled surfaces 304a, b across the respective channel 306a, b of the jaw 300, 302 from the elongate couplers 295a, b. The angled surfaces 304a, b may be angled to increase in height towards a distal direction of the clip 292 and may be angled to extend downward transverse to the plane of movement (e.g., opening and closing) of the clip 292. The angled surfaces 304a, b may be angled to extend downward transverse to the plane of movement of the clip 292 in a direction towards the central space of the clip 292 between the jaws 300, 302. Such angulation may match a chamfer 305a, b of the rails indicated in FIG. 30. The angulation of the angled surfaces 304a, b may aid to stabilize the clip 292 when slidably engaged with the arms 294, 296. The clip 292 may otherwise be configured similarly as the clip 30.

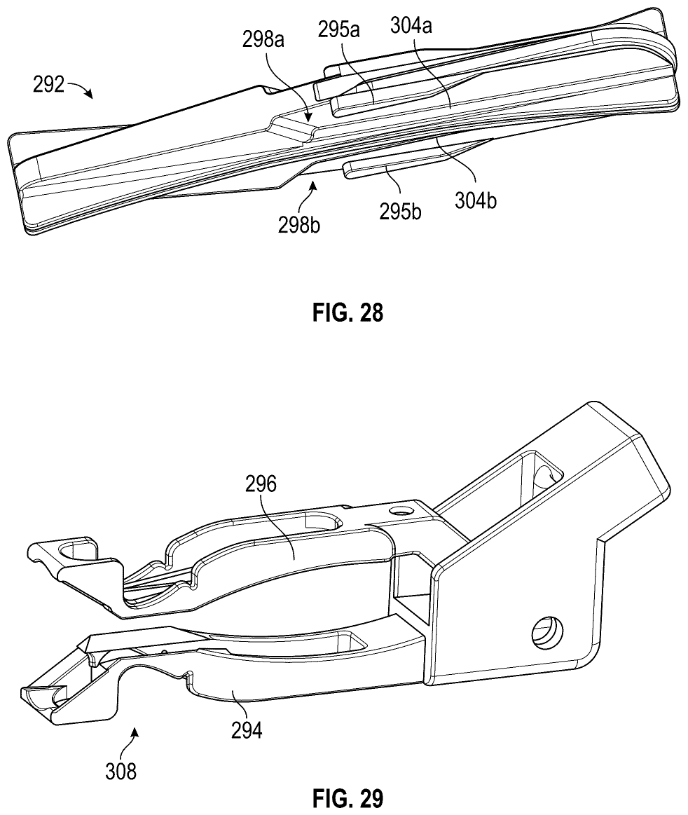

FIG. 28 illustrates a top perspective view of the clip 292.

FIG. 29 illustrates a perspective view of the engagement portion 308. Features of the expansion apparatus 140 may be utilized with the examples of FIGS. 27-35 unless stated otherwise.

The respective arms 294, 296 of the engagement portion 308 may include angled rails 310a, b that may match an angle of the tapered elongate couplers 295a, b of the clip 292 and the angled surfaces 304a, b of the clip 292. FIG. 31, for example, illustrates a rail surface 312a of the rail 310b that is angled to match the angulation of one of the tapered elongate couplers 295a, b. The rail 310b may include the chamfer surface 305b that may match the angulation of one of the angled surfaces 304a, b of the clip 292. Such a configuration may allow for more secure sliding engagement with the clip 292. Each arm 294, 296 may be configured similarly.

The retention mechanism may operate to retain the clip 292 to the engagement portion 308 to impede sliding release of the clip 292 from the arms 294, 296 in a similar manner as discussed in regard to FIGS. 4-20. For example, the arms 294, 296 may be positioned in an at least partially opened configuration to retain the clip 292 to the engagement portion 308. An expansion force may be applied by the arms 294, 296 to the clip 292 in a similar manner as discussed herein. Other features disclosed in regard to the examples of FIGS. 4-20 may be utilized with the examples of FIGS. 27-35, including use of selectively actuatable retention mechanism (e.g., via a control device). The examples of FIGS. 27-35 may utilize the barrier 290 in combination or in lieu of the features of the examples of FIGS. 4-20.

FIG. 32 illustrates the barrier 290 positioned at a distal end portion of the second arm 296. One or more of the barriers 290 may be positioned to contact the contact surfaces 297a, b to impede sliding release of the clip 292 from the arms 294, 296. FIG. 33, for example, illustrates the barrier 290 contacting the contact surface 297a. The arms 294, 296 may be positioned in an at least partially opened configuration, as discussed herein, which causes the barrier 290 to be in contact with the contact surface 297a. The retention mechanism accordingly may be in a retention configuration.

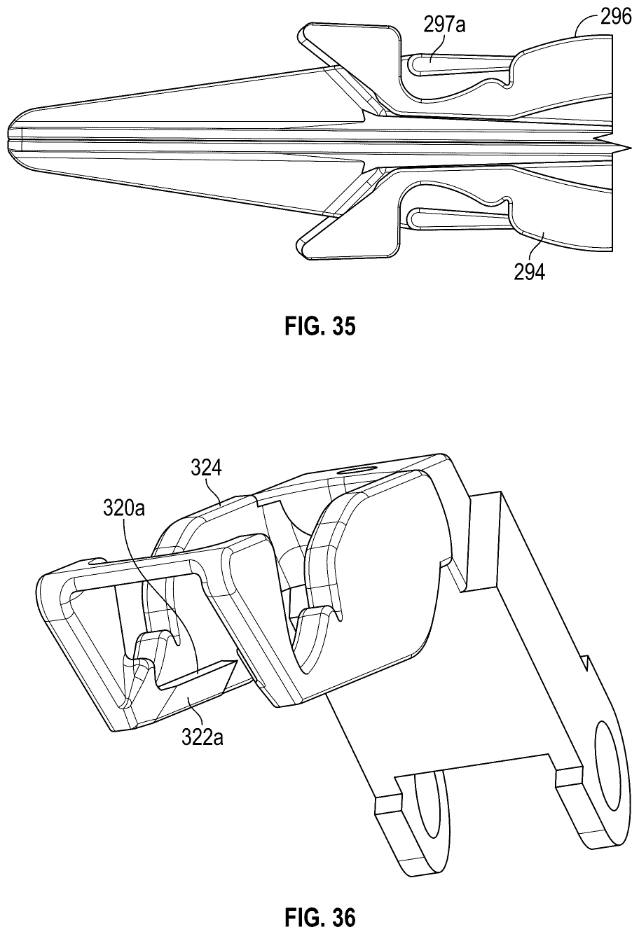

Upon the retention mechanism being actuated to the release configuration, the arms 294, 296 may pivot closer to each other (e.g., in a closed configuration represented in FIG. 5 for example). FIG. 34 illustrates such a configuration. The barrier 290 accordingly displaces relative to a portion of the clip (e.g., the contact surface 297a) to allow for sliding release of the clip 292 from the arms 294, 296. The barrier 290 has displaced to position the contact surface 297a within an opening that allows for sliding release of the clip 292 from the arms 294, 296. FIG. 35 illustrates a resulting configuration of the arms 294, 296.

In examples, the displacement of the barrier 290 may occur in response to a force of occlusion being applied to the clip 292. The clip 292, upon occluding, may be pressed outward from the occluded body (due to the space provided by the occluded body between the jaws 300, 302 of the clip 292). The contact surfaces 297a, b of the jaws of the clip 292 may be raised above the barrier 290 due to this occlusive force, which may allow for sliding release of the clip 292 upon occluding a body.

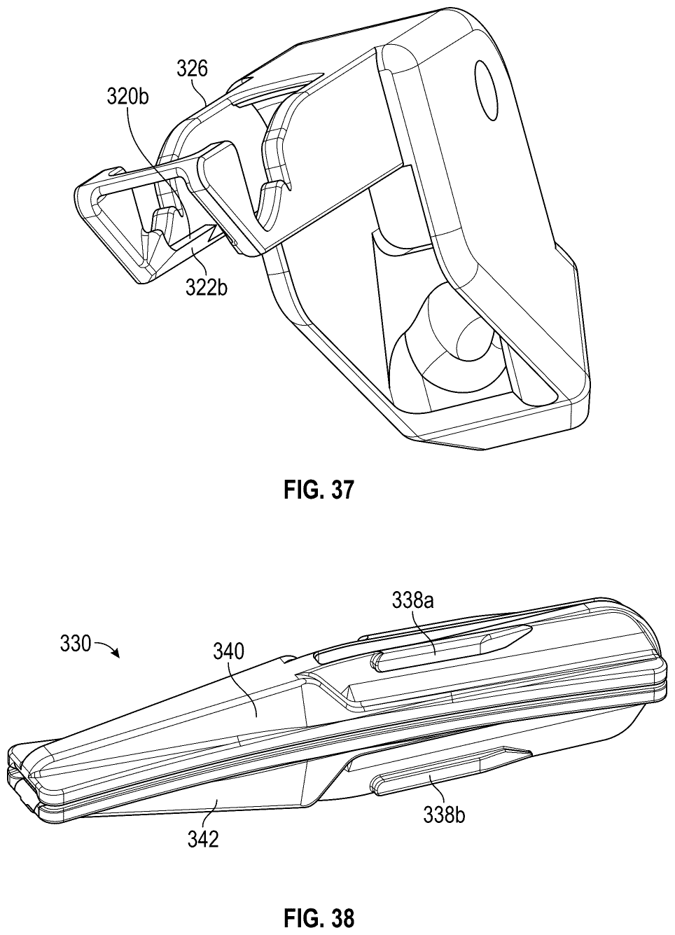

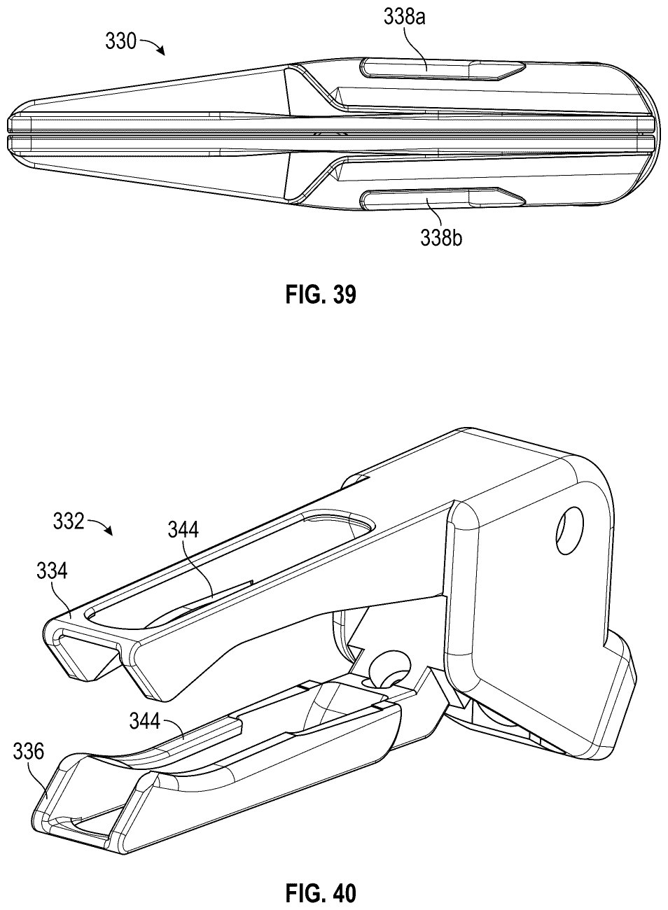

Various other configurations of the arms 294, 296 may be provided in examples. FIG. 36, for example, illustrates a variation of the second arm 296 including linear rail surfaces 320a and linear chamfer surfaces 322a. The features of the second arm 296 may otherwise be utilized with the second arm 324. FIG. 37 illustrates a configuration of a first arm 326 that may be utilized having similar linear rail surfaces 320b and linear chamfer surfaces 322b. The features of the first arm 294 may otherwise be utilized with the first arm 326. The configuration of the linear rail surfaces and chamfer surfaces may be utilized with a clip 330 as shown in FIGS. 38 and 39 for example, among other forms of clips.

The features of FIGS. 27-37 may be utilized solely or in combination with any other example herein.

Other configurations of retention mechanisms may be utilized in examples. FIGS. 38-45, for example, illustrate an example in which a control device is not utilized to actuate the retention mechanism. An occlusive force upon the clip 330 rather releases the clip 330 from the engagement portion 332 (marked in FIG. 40) and allows for sliding release of the clip 330 from the arms 334, 336. The retention mechanism may retain the clip 330 to the engagement portion 332 when the occlusive force is not applied to the clip 330 (e.g., when there is no tissue between the jaws of the clip 330).

FIGS. 38 and 39 illustrate an example of a clip 330 that may be utilized. The clip 330 may include elongate couplers 338a, b that have linear contact surfaces that extend along the length of the respective jaw 340, 342. Such a configuration may differ from the tapered elongate couplers 295a, b of the clip 292 shown in FIG. 27 for example. FIG. 39 illustrates a side view of the clip 330.

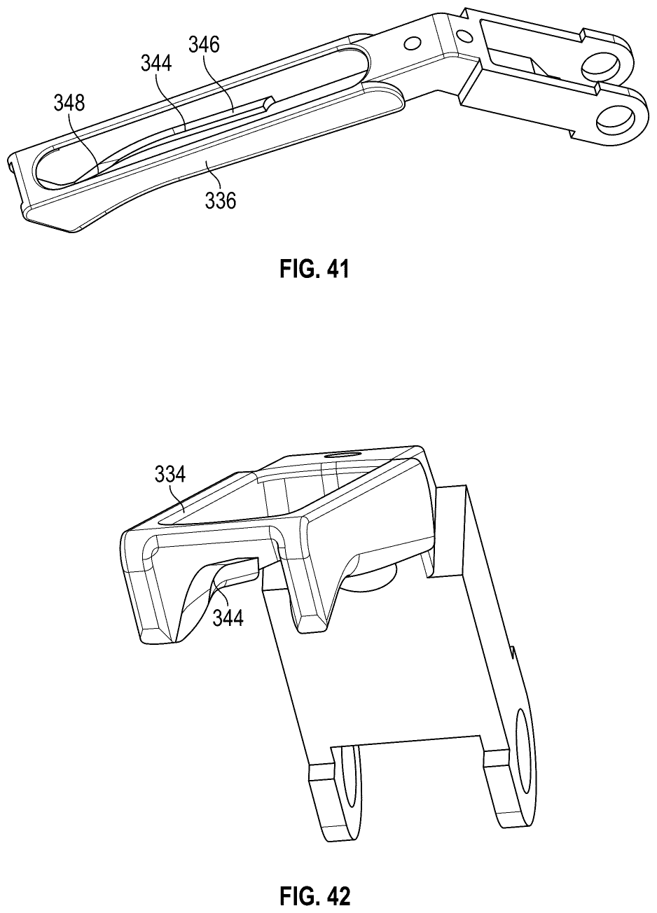

FIG. 40 illustrates a perspective view of the engagement portion 332. Each arm 334, 336, may include rails 344 that may include a linear portion 346 (marked in FIG. 41) and a ramp portion 348. The ramp portion 348 may be configured to improve the sliding engagement of the clip 330 with the arms 334, 336. The linear portion 346 may slide along the respective elongate couplers 338a, b to support the elongate couplers 338a, b similar to other configurations of rails disclosed herein.

FIG. 41 illustrates a perspective view of the second arm 336. FIG. 42 illustrates a perspective view of the first arm 334. The clip 330 may slidably engage with the arms 336, 334 with a friction force or interference fit securing the clip 330 to the arms 336, 334. The friction force or interference fit may be provided by the engagement of the rails 344 and the elongate couplers 338a, b.

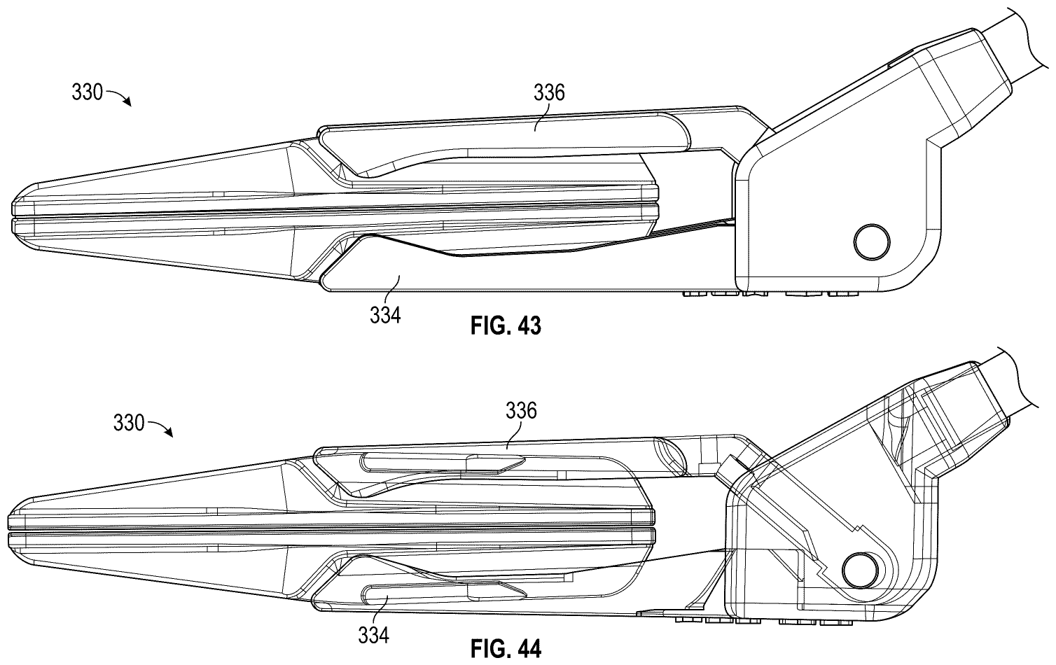

FIGS. 43 and 44, for example, illustrate the clip 330 engaged with the arms 336, 334. The rails 344 may slide into the channels of the respective jaws 340, 342.

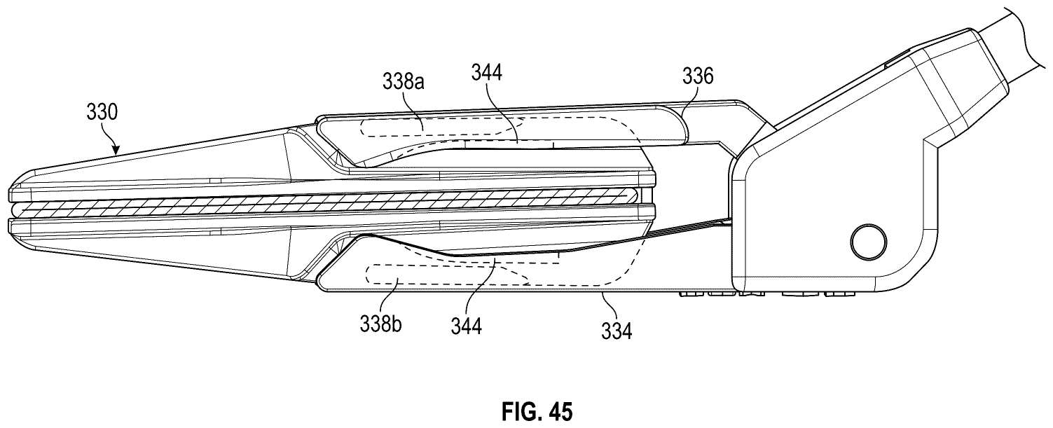

Upon deployment of the clip 330 to a body to be occluded, the occlusive force applied to the clip 330 (e.g., tissue between the jaws of the clip 330) may clear the elongate couplers 338a, b from the rails 344 as represented in FIG. 45. A space may be provided between the elongate couplers 338a, b and the rails 344. The friction force or interference fit between the rails 344 and the elongate couplers 338a, b may be reduced. The clip 330 may be able to slidably release from the arms 336, 334. The retention mechanism may operate without use of a separate control device for actuating the retention mechanism. The occlusive force upon the clip 330 may allow the clip 330 to release from the arms 336, 334.

The retention mechanism may operate automatically, without use of a separate control device (e.g., a lever or other form of control device).

The features of FIGS. 38-45 may be utilized solely or in combination with any other example herein.

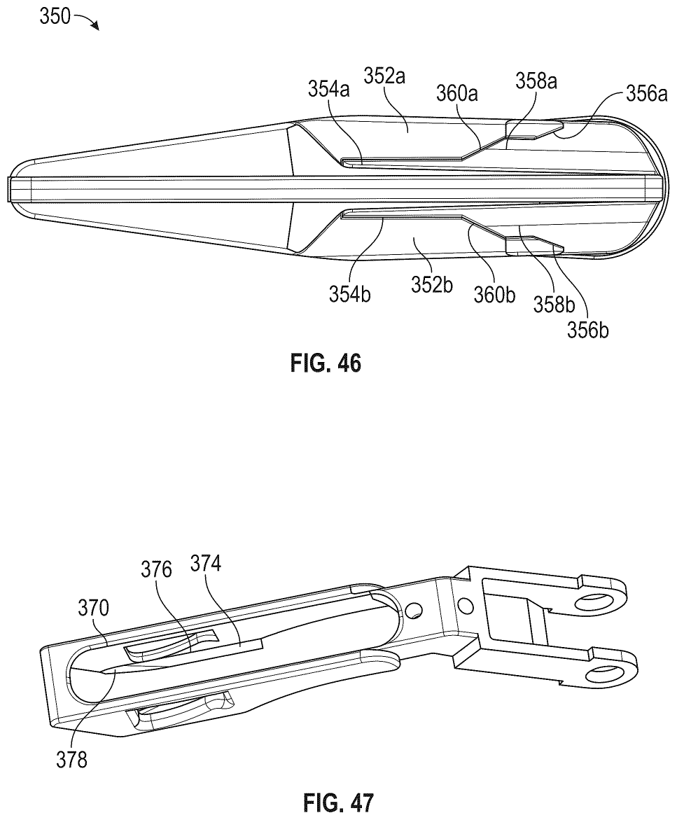

Variations in the configuration of a clip utilized may be provided in examples. FIG. 46, for example, illustrates a side view of a clip 350 that may be utilized in examples herein. The clip 350 may include the features of the clip 330 unless stated otherwise. The clip 350 may include elongate couplers 352a, b having tapered surfaces for guiding rails of an engagement portion of an expansion apparatus into distal channels 354a, b. The elongate couplers 352a, b, for example, may include first tapered surfaces 356a, b or proximal tapered surfaces that taper to respective proximal channels 358a, b. The elongate couplers 352a, b may include second tapered surfaces 360a, b or distal tapered surfaces that taper to the distal elongate channels 354a, b. An opposite side of the clip 350 may include similar elongate couplers.

The proximal channels 358a, b may be wider or have a greater height than the distal channels 354a, b. The distal channels 354a, b may have a greater length than the proximal channels 358a, b. The second tapered surfaces 360a, b may comprise a transition between the relatively wider proximal channels 358a, b and the relatively narrower distal channels 354a, b.

The first tapered surfaces 356a, b and the second tapered surfaces 360a, b may both be angled to direct the rails of the engagement portion into the distal channels 354a, b upon engagement with the engagement portion of an expansion apparatus.

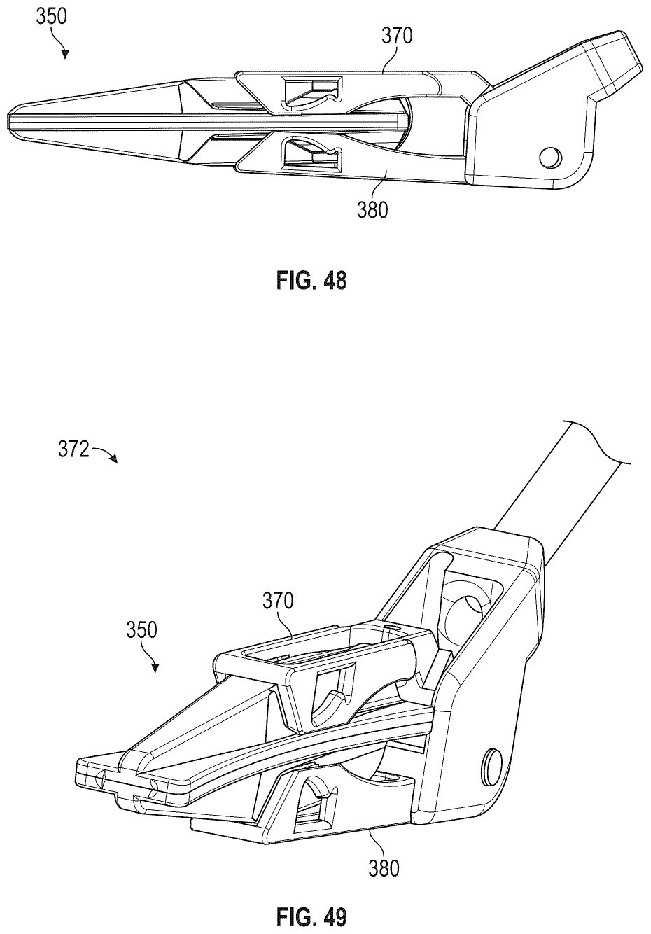

FIG. 47 illustrates a perspective view of an arm 370 of an engagement portion 372 (marked in FIG. 48) of an expansion apparatus that may be utilized. The arm 370 may be configured similarly as the arm 336 shown in FIG. 41 unless stated otherwise. The arm 370, for example, may include rails 374 that may include a linear portion 376 and a ramp portion 378. The ramp portion 378 may comprise a tapered surface of the rail 374 for engagement with the tapered surfaces 356a, b, 360a, b of the elongate couplers 352a, b. The ramp portion 378 and linear portion 376, however, may be linear with each other, to improve insertion into the linear distal channels 354a, b. The ramp portions 378 may slide along the tapered surfaces 356a, b, 360a, b of the elongate couplers 352a, b to insert into the distal channels 354a, b.

FIG. 48, for example, illustrate the clip 350 being slid along the rails 374 for engagement with the engagement portion 372 of the expansion apparatus. The arm 380 may include a rail that is similarly configured as the rail of the arm 370.

FIG. 49 illustrates the clip 350 engaged with the engagement portion 372. The rails 374 (marked in FIG. 47) of the arms 370, 380 are positioned within the distal channels 354a, b of the clip 350. The clip 350 is secured in position by the engagement with the rails 374. The clip 350 may slidably engage with the arms 370, 380 with a friction force or interference fit securing the clip 350 to the arms 370, 380. The friction force or interference fit may be provided by the engagement of the rails 374 and the elongate couplers 352a, b.

In examples, a compressive force applied by the arms 370, 380 to the clip 350 may retain the clip 350 to the engagement portion 372.

In examples, an occlusive force upon the clip 350 may release the clip 350 from the engagement portion 372 in a similar manner as described in regard to FIGS. 40-45. A control device is not utilized to actuate the retention mechanism. The occlusive force allows for sliding release of the clip 350 from the arms 370, 380. The retention mechanism may retain the clip 350 to the engagement portion 372 when the occlusive force is not applied to the clip 350 (e.g., when there is no tissue between the jaws of the clip 350).

Upon deployment of the clip 350 to a body to be occluded, the occlusive force applied to the clip 350 (e.g., tissue between the jaws of the clip 350) may clear the elongate couplers 352a, b from the rails 374 in a similar manner as represented in FIG. 45. A space may be provided between the elongate couplers 352a, b and the rails 374. The friction force or interference fit between the rails 374 and the elongate couplers 352a, b may be reduced. The clip 350 may be able to slidably release from the arms 370, 380. The retention mechanism may operate without use of a separate control device for actuating the retention mechanism. The occlusive force upon the clip 350 may allow the clip 350 to release from the arms 370, 380.

The retention mechanism may operate automatically, without use of a separate control device (e.g., a lever or other form of control device).

The features of FIGS. 46-49 may be utilized solely or in combination with any other example disclosed herein.

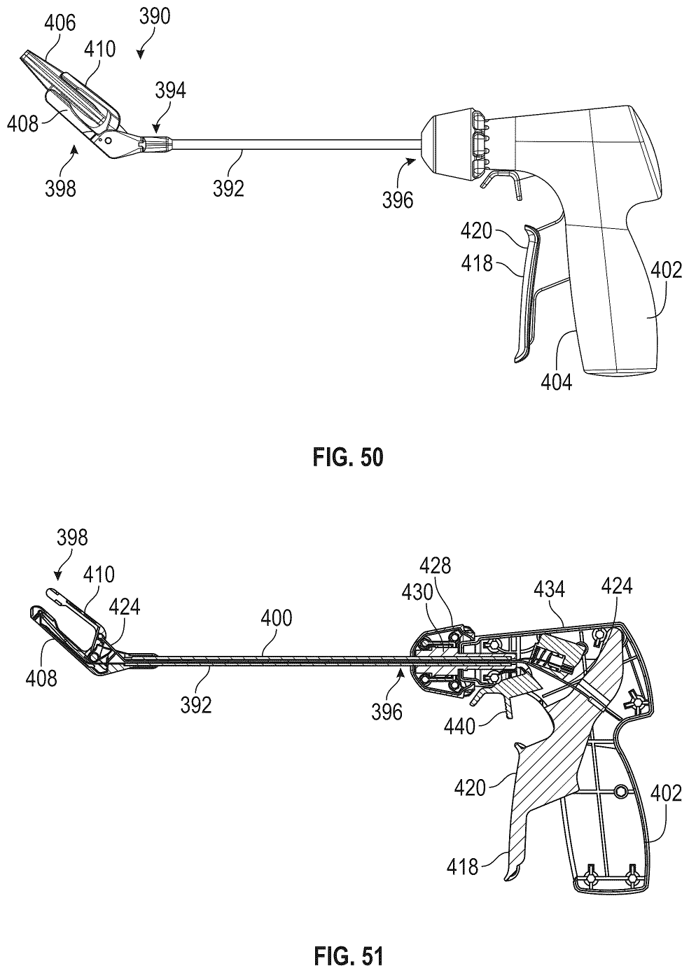

FIG. 50 illustrates a side view of an expansion apparatus 390 that may be utilized in examples herein. The expansion apparatus 390 may include the features of the expansion apparatus 140 or any other form of expansion apparatus disclosed herein unless stated otherwise. The expansion apparatus 390 may include an elongate shaft 392 having a first end portion 394 or distal end portion and a second end portion 396 or proximal end portion. The elongate shaft 392 may comprise a rigid shaft or may comprise a flexible shaft in examples. In an example in which the elongate shaft 392 is a flexible shaft, the elongate shaft 392 may be deflected in one more planes to position the engagement portion 398 in a desired orientation for deployment or capture of a clip.

The elongate shaft 392 may be configured for one or more components of a control mechanism to pass therethrough, to extend to the engagement portion 398 in examples. The elongate shaft 392 may include an interior lumen 400 (marked in FIG. 51) for example.

The expansion apparatus 390 may include a handle 402 in examples. The handle 402 may be positioned at the second end portion 396 or proximal end portion of the elongate shaft 392. The handle 402 may include a grip portion 404 for a user to grip in examples.

The expansion apparatus 390 may include an engagement portion 398. The engagement portion 398 may be configured to engage the clip 406 in examples. The engagement portion 398 may be positioned at the first end portion 394 of the elongate shaft 392 in examples.

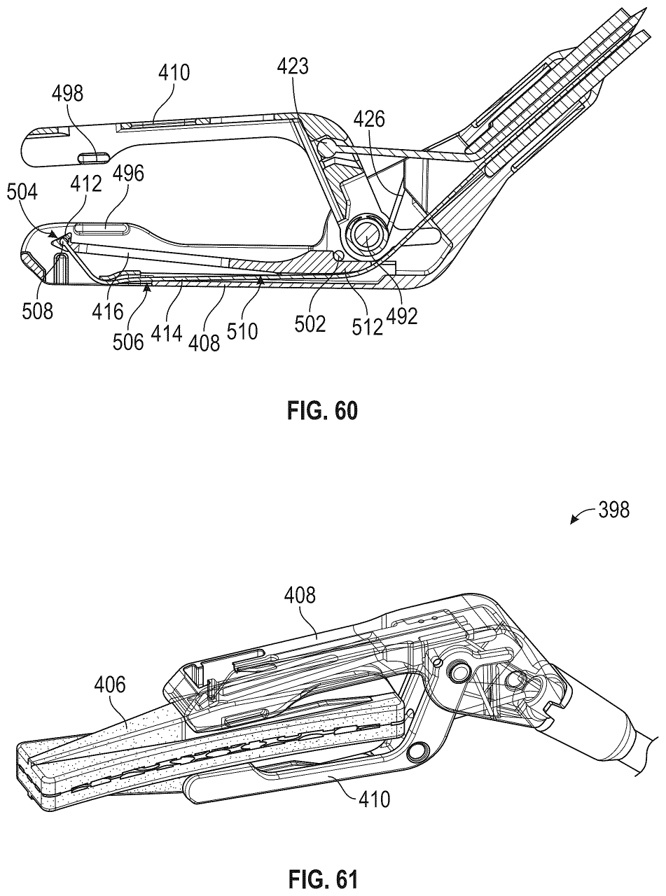

The engagement portion 398 may be configured similarly as the engagement portion 152 of the expansion apparatus 140 unless stated otherwise. The engagement portion 398 may have at least two arms. The engagement portion 398 may have a first arm 408 and a second arm 410. The engagement portion 398 may include a retention mechanism having a protrusion 412 that is actuatable via a tether 414 (shown in FIG. 60). The protrusion 412 may be coupled to a lever arm 416 (shown in FIG. 60). The protrusion 412 may be configured to engage at least a portion of the left atrial appendage clip 406 to impede sliding release of the left atrial appendage clip 406 from the first arm 408 and the second arm 410.

Referring to the cross sectional view of FIG. 51, a control mechanism may be utilized for controlling opening and closing of the first arm 408 relative to the second arm 410. The control mechanism may include a control device 418. The control device 418 may be positioned upon the handle 402 in examples, or may be alternatively positioned in examples. The control device 418 may comprise a control lever arm 420 in examples. The control lever arm 420 may include a trigger or grip portion of the control device 418 in examples, or may have other configurations. The control lever arm 420 may extend into an interior of the handle 402 as shown in FIG. 51, or other configurations may be utilized in examples.

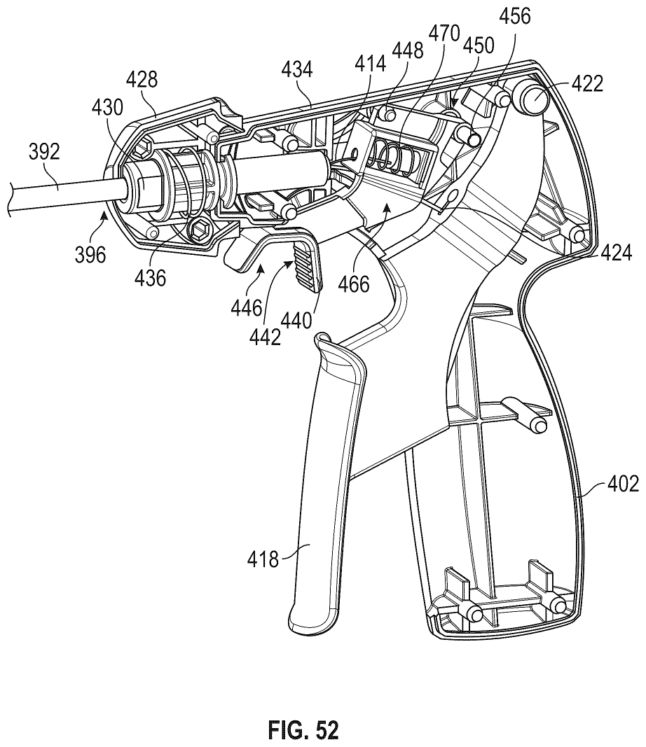

The control lever arm 420 may be configured to pivot, and may be pivotally coupled to the handle 402 at a pivot 422 (marked in FIG. 52). The control lever arm 420 may rotate about the pivot 422. In examples, other configurations of control devices 418 may be utilized.

The control mechanism may include a tether 424 in examples. The tether 424 may extend from the control device 418 to the engagement portion 398 of the expansion apparatus 390 in examples. Other configurations may be utilized in examples. The tether 424 may have a proximal end portion that may couple to the control device 418 and may have a distal end portion that may couple to the second arm 410. FIG. 60, for example, illustrates a coupling point 423 of the distal end portion of the tether 424 to the second arm 410. The tether 424 may extend within the interior lumen 400 of the elongate shaft 392 in examples. Other configurations may be utilized in examples.

A retraction or other retracting movement of the control device 418 may provide a tension or retracting movement upon the tether 424. The tether 424 may retract and accordingly provide a tension or retraction force against the second arm 410. A pivotal coupling of the second arm 410 may allow the second arm 410 to pivot and move to the opened configuration. Upon release of the control device 418, the force applied by a spring 426 (marked in FIG. 60) may cause the second arm 410 to return to the closed configuration. As such, selective opening or closing of the arms 408, 410 may result.

Referring to FIG. 52, in examples, a rotation mechanism may be utilized for controlling rotation of the engagement portion 398 about a longitudinal axis of the elongate shaft 392. The rotation mechanism, for example, may include a control device 428 in the form of a rotatable knob that may be utilized to rotate the engagement portion 398 about the longitudinal axis of the elongate shaft 392. The control device 428 may engage an adaptor 430 that may be coupled to the second end portion 396 or proximal end portion of the elongate shaft 392. The adaptor 430 may be fixedly coupled to the elongate shaft 392. The control device 428 may be rotated about the longitudinal axis of the elongate shaft 392 to rotate the adaptor 430 and accordingly rotate the elongate shaft 392.

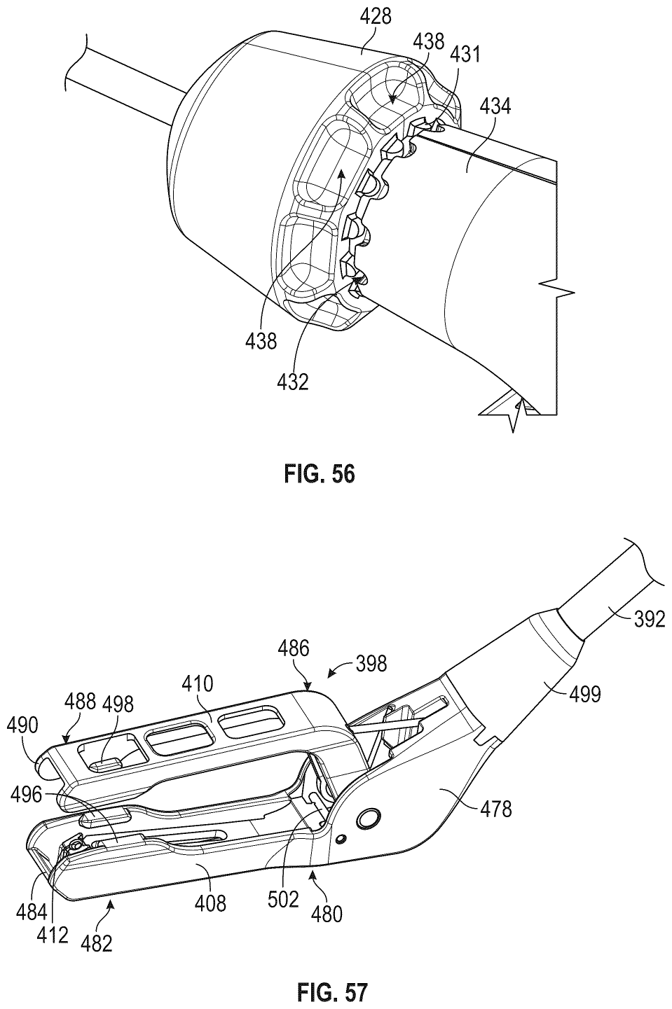

Referring to FIG. 56, the rotation mechanism may include a lock for selectively locking a rotational position of the engagement portion about the longitudinal axis of the elongate shaft. The lock, for example, may comprise engagement surfaces 431 of the control device 428 for engagement with engagement surfaces 432 of a housing 434. The housing 434, may comprise the housing of the handle 402 in examples.

The engagement surfaces 431 of the control device 428 may comprise a plurality of spaced protrusions. The engagement surfaces 432 of the housing 434 may comprise a plurality of spaced recesses. The spaced protrusions may be configured to engage the spaced recesses. The spaced protrusions may be equally spaced from each other, with the spaced recesses having the same spacing. In examples, the configuration of the spaced protrusions and the spaced recesses may be inverted such that the housing 434 includes the spaced protrusions and the control device 428 includes the spaced recesses.

The control device 428 may be axially slidable both distal and proximal and may be spring biased with a spring 436 (marked in FIG. 52) to a proximal position. The proximal position may be a locked position, with the engagement surfaces 431, 432 engaging each other. A distal force applied to the control device 428 may disengage the engagement surfaces 431, 432 from each other. The control device 428 in the distal position is shown in FIG. 56, with the engagement surfaces 431, 432 disengaged from each other. In such a position, the control device 428 may be freely rotated about the longitudinal axis of the elongate shaft 392. A removal of the distal force may cause the spring 436 (marked in FIG. 52) to press the control device 428 proximally to the locked configuration, in which the engagement surfaces 431, 432 engage each other. The spacing of the protrusions and the recesses of the engagement surfaces 431, 432 may comprise datum points providing defined angles for rotational orientation of the elongate shaft 392. The control device 428 may be rotated to the defined angles as desired by a user.

In examples, the control device 428 may be configured to be operable with a single finger. For example, a user may grip the grip portion 404 of the handle 402 with the user's thumb, and middle (third finger), ring (fourth finger), and pinky finger (fifth finger). The index finger (second finger) may remain free to contact the control device 428 and rotate the control device 428. A distal force may be applied by the index finger to the control device 428 to disengage the engagement surfaces 431, 432 from each other and to rotate the control device 428 to a desired position. The distal force may be released to cause the engagement surfaces 431, 432 to reengage. As such, single finger operation of the control device 428 may be provided, with a single hand gripping the grip portion 404 of the handle 402. In examples, the control device 428 may include indentations 438 for receiving a tip of a finger for operation of the control device 428. In examples, other fingers than the index finger, or multi-finger operation may be utilized.

In examples, a retention mechanism may be utilized that may be configured to retain the clip 406 to the engagement portion 398 to impede sliding release of the clip 406 from the first arm 408 and the second arm 410. The retention mechanism may have a variety of forms in examples. The retention mechanism may be utilized to secure the clip 406 to the engagement portion 398 such that the clip 406 does not release from the engagement portion 398 until desired. For example, upon approach to an implantation site or upon assembly or handling of the expansion apparatus 390, the clip 406 may be prevented from prematurely releasing from the engagement portion 398 by undesirably sliding longitudinally off of the engagement portion 398. A reduced possibility of unintended movement or force dislodging the clip 406 from the engagement portion 398 may result. The retention mechanism may be utilized to allow a user to release the clip 406 from the engagement portion 398 at a desired time. For example, a user may place the clip 406 in position at a desired implantation site, and then actuate the retention mechanism to release the clip 406 upon a final position being determined. As such, a premature release of the clip 406 may be reduced. In examples in which the expansion apparatus 390 is utilized for recapture or repositioning of the clip 406, the retention mechanism may be utilized to confirm securement of the clip 406 to the engagement portion 398. A retention mechanism may comprise a protrusion 412 that is actuatable via a tether 414 (shown in FIG. 60). The protrusion 412 may be coupled to a lever arm 416 (shown in FIG. 60).

FIG. 52 illustrates a perspective view of a control device 440 for the retention mechanism. The control device 440 may be operable by a user to selectively actuate the retention mechanism. The control device 440 as shown in FIG. 52 may comprise a lever, although other configurations may be utilized (e.g., a slider, a knob, among others). One or more of a lever, a slider, or a knob may be utilized as desired. The control device 440 may be operated by the user to actuate the retention mechanism between a release configuration and a retention configuration as desired.

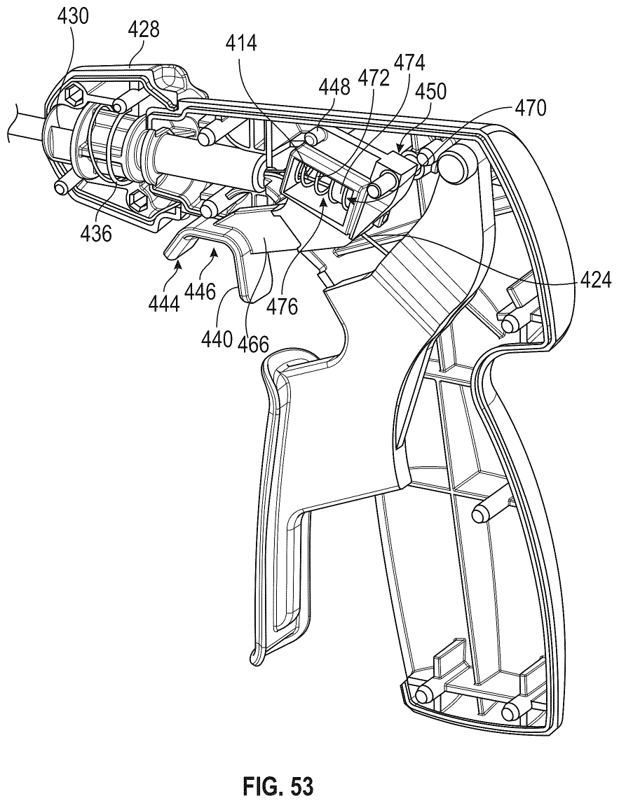

The control device 440 may include a first contact surface 442 (marked in FIG. 52) that may be configured to be retracted proximally to move the retention mechanism to the release configuration. The control device 440 may include a second contact surface 444 (marked in FIG. 53) that may be configured to be advanced distally to move the retention mechanism to the retention configuration. The first contact surface 442 and the second contact surface 444 may be positioned opposed to each other such that a single finger (e.g., an index finger or middle finger) may be utilized to selectively actuate the control device 440 between the release configuration or the retention configuration. The first contact surface 442 may face the second contact surface 444 (with a “U” shape), with the first contact surface 442 and second contact surface 444 bounding a well 446 for receiving a finger.

The control device 440 may be pivotally coupled to the handle 402 at a pivot 448. The control device 440 may pivot between the retention or distal position and a release or proximal position upon operation of the control device 440. In examples, a lock mechanism 450 may be utilized for locking the retention mechanism in the retention configuration and locking the retention mechanism in the release configuration.

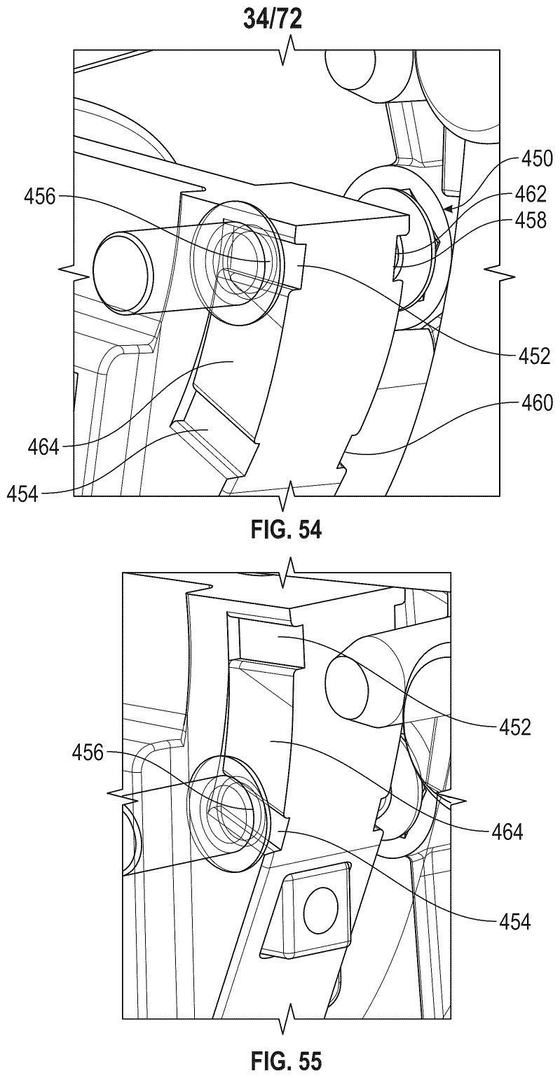

FIG. 54 illustrates a close up view of a lock mechanism 450 that may be utilized. The lock mechanism 450 may include a first recess 452 and a second recess 454 and a protrusion 456 for selectively engaging the first recess 452 or the second recess 454. The protrusion 456 is shown positioned in the first recess 452 in the retention configuration in FIG. 54. In examples, multiple recesses or protrusions may be utilized, for example, opposed recesses 458, 460 (corresponding to respective recesses 452, 454) and an opposite protrusion 462 (corresponding to the protrusion 456) may be utilized.

In examples, the protrusion 456 may comprise a detent or a spring biased protrusion configured to disengage from the first recess 452 upon a force being applied to the protrusion 456. Other forms of protrusions may be utilized in examples.

The protrusion 456 may be configured to slide along a channel 464 that extends between the first recess 452 and the second recess 454. The channel 464 may have an arc shape corresponding to a path of rotation of the control device 440 upon pivoting about the pivot 448 between the release configuration and the retention configuration.

FIG. 55, for example, illustrates the control device 440 having pivoted to the release configuration. The protrusion 456 is held within the second recess 454. The protrusion 456 may be retained within the second recess 454 to thus lock the retention mechanism in the release configuration. The protrusion 456 may be retained within the second recess 454 until the user pivots the control device 440 to the retention configuration (with the protrusion 456 held in the first recess 452).

In examples, the recesses 452, 454 and channel 464 of the lock mechanism 450 may be positioned on an interior housing portion 466 (marked in FIG. 52) of the control device 440 that is disposed within the handle 402. The protrusion 456 may be coupled to the housing 434 of the handle 402. Alternatively, the recesses 452, 454 and channel 464 may be positioned on the housing 434 of the handle 402 and the protrusion 456 may be positioned on the interior housing portion 466. Other configurations may be utilized in examples.

In examples, the retention mechanism may include a force dampener 470 (marked in FIG. 52) that may be configured to dampen a force applied to the tether 414 of the retention mechanism. The force dampener 470 may have a variety of forms in examples and may include a spring 472 (marked in FIG. 53) for applying a force to a displacement body 474. The displacement body 474 may be coupled to the proximal end portion of the tether 414. The displacement body 474 may be positioned within a slide channel 476, and the spring 472 may be positioned within the slide channel 476 for applying a proximal force to the displacement body 474. The spring 472 accordingly may bias the displacement body 474 and the tether 414 in a proximal direction.

In examples, the spring 472, displacement body 474, and slide channel 476 may be positioned on the interior housing portion 466 of the control device 440. As such, upon movement of the control device 440, the entire assembly of the spring 472, displacement body 474, and slide channel 476 may move along with the control device 440. The displacement body 474 may correspondingly move the tether 414 to actuate the retention mechanism. However, upon a distal force being applied to tether 414, the spring 472 may compress to reduce the tension in the tether 414. Such a feature may reduce the possibility of damage to the tether 414 or otherwise undesired tension in the tether 414. The tension may be produced due to a deflection of the elongate shaft 392 (e.g., bending the elongate shaft 392 to orient the elongate shaft 392 in a desired position for deployment of the clip 350). Other forms of force dampeners may be utilized in examples. In examples, the use of a force dampener may be excluded.

FIG. 57 illustrates a perspective view of the engagement portion 398 of the expansion apparatus 390. The first arm 408 and second arm 410 may be configured similarly as the respective first arm 154 and second arm 156 of the expansion apparatus 140 unless stated otherwise. The first arm 408 may be configured to engage a first jaw of the clip 406, and the second arm 410 may be configured to engage a second jaw of the clip 406. The first arm 408 may be configured to move relative to the second arm 410 to open or close the clip 406.

The arms 408, 410 may each extend from a base 478. The first arm 408 may have a proximal end portion 480 and a distal end portion 482. The first arm 408 may extend longitudinally and may be for sliding engagement with the first jaw of the clip 406. The proximal end portion 480 may be fixedly coupled to the base 478 in examples. The first arm 408 may extend longitudinally from the proximal end portion 480 to the distal end portion 482. The distal end portion 482 may comprise a distal tip 484 of the first arm 408.

The second arm 410 may have a proximal end portion 486 and a distal end portion 488. The second arm 410 may extend longitudinally and may be for sliding engagement with a second jaw of the clip 406. The proximal end portion 486 may be pivotally coupled to the base 478 in examples. The second arm 410 may extend longitudinally from the proximal end portion 486 to the distal end portion 488. The distal end portion 488 may comprise a distal tip 490 of the second arm 156.

The second arm 410 may couple to the base 478 through use of a pivot axle 492 (marked in FIG. 59), in a similar manner as disclosed in regard to the second arm 156. Further, a spring 426 (marked in FIG. 59) may be utilized in a similar manner as the spring 178. Other configurations may be utilized in examples.

The first arm 408 and second arm 410 may include respective rails 496, 498 that may operate in a similar manner as the rails 184a, b, 191 of the arms 154, 156.

The base 478 may couple to the elongate shaft 392 via an overmolded body 499. The overmolded body 499 may fixedly couple the base 478 to the elongate shaft 392 such that the base 478 rotates or otherwise moves with the movement of the elongate shaft 392. As such, movement of the elongate shaft 392 may control the position of the engagement portion 398 as desired.

The engagement portion 398 may include a retention mechanism that may include the features of the retention mechanism of FIGS. 21-23 unless stated otherwise. Referring to FIGS. 57-60, the retention mechanism may include a protrusion 412 that is actuatable via a tether 414. The protrusion 412 may be coupled to a lever arm 416. The retention mechanism provides a force from the arm 408 to retain the clip to the engagement portion 398 to impede sliding release of the clip from the arms 408, 410.

The protrusion 412 is configured to engage at least a portion of the clip 406 to impede sliding release of the arms 408, 410. The protrusion 412 may comprise a hook. The hook may be configured to enter a recess 500 (marked in FIG. 63) on the clip 406. The recess 500 may be shaped to receive the hook. The hook may create a mechanical interference with the clip 406 upon engaging the clip 406.

The protrusion 412 may be pivotally coupled to the engagement portion 398 at a pivot 502. The protrusion 412 may comprise a portion of a lever arm 416 configured to pivot about the pivot 502 upon a retraction force being applied by the tether 414. The lever arm 416 may extend along a length of the first arm 408, although other configurations may be utilized as desired. The protrusion 412 may have an angled surface or tapered surface 504 (marked in FIG. 60) to allow the protrusion 412 to automatically retract from the clip 406 upon the clip 406 being inserted between the arms 408, 410 and slidably engaged with the arms 408, 410.

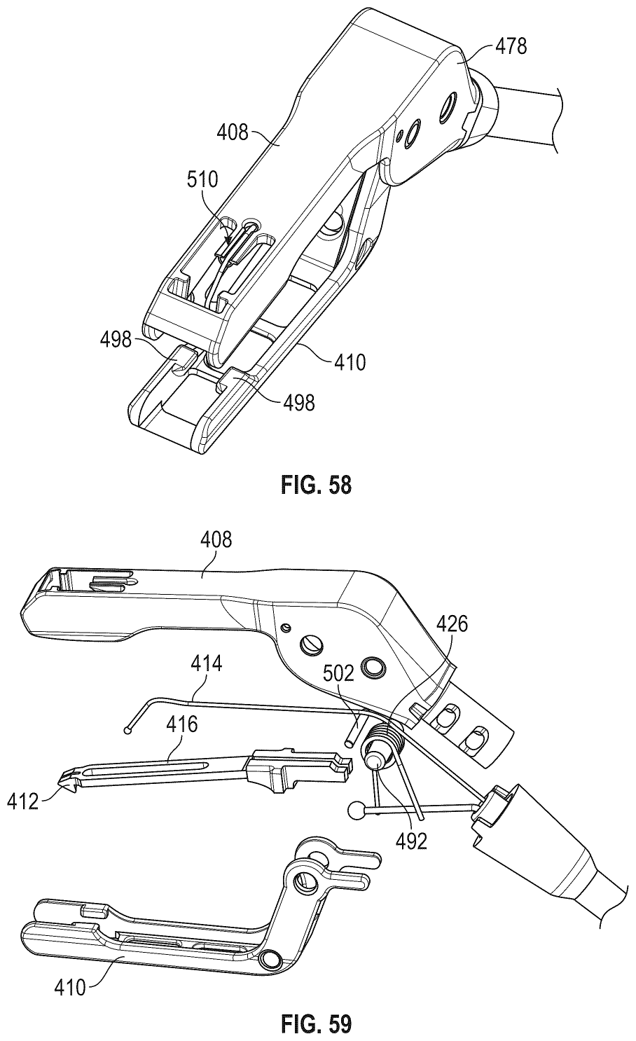

Referring to FIG. 60, in examples, a routing assembly 506 may be utilized to route the tether 414 to a coupling point 508 with the lever arm 416. The routing assembly 506, for example, may comprise a tether channel 510 in the first arm 408 that may extend the tether 414 over the lever arm 416 and to the coupling point 508 at the distal end of the lever arm 416. The tether channel 510 may direct the tether 414 to the coupling point 508 from an outer or upper surface of the first arm 408 (as marked in FIG. 58).

The routing assembly 506 may direct the tether 414 such that the tether 414 extends over a proximal end portion 512 or opposite lever portion (opposite of the pivot 502) of the lever arm 416. The tether 414 is routed such that a tension applied by the tether 414 accordingly applies a force to the proximal end portion 512 to produce pivotal motion about the pivot 502. The lever arm 416 pivots from the retention configuration shown in FIG. 60 to a release configuration. The routing assembly 506 directs the tether 414 to the coupling point 508 at distal end of the lever arm 416, thus applying a leverage force to the lever arm 416. Rotation of the lever arm 416 results. Other configurations for routing the tether 414 may be utilized in examples.

The lever arm 416 may be actuated through control of the control device 440 as disclosed herein. A retraction of the control device 440 to the release configuration produces a tension in the tether 414 that correspondingly applies a force to the lever arm 416 to actuate the protrusion 412 from the retention configuration to the release configuration. An advancement or distal movement of the control device 440 to the retention configuration applies a distal force to the tether 414 that correspondingly advances the lever arm 416 such that the protrusion 412 moves to the retention configuration. The protrusion 412 accordingly is selectively actuatable from a retention configuration to a release configuration in which the protrusion 412 allows for sliding release of the clip 406 from the arms 408, 410. FIG. 61, for example, illustrates the clip 406 retained by the engagement portion 398 of the expansion apparatus 390. The retention mechanism may be utilized for deployment or capture (e.g., recapture) of a deployed clip.

Various other configurations of retention mechanisms may be utilized in examples.

In examples, the clip 406 may be pre attached to the expansion apparatus prior to shipping and delivery to a surgical facility, or the clip 406 may be attached to the expansion apparatus on-site. The clip 406 may be attached to the expansion apparatus in a sterilization process, which may comprise an e-beam sterilization process or other process (e.g., gaseous sterilization). The clip 406 attached to the expansion apparatus may be retained within packaging for the sterilization process.

The features of FIGS. 50-61 may be utilized solely or in combination with any other example herein.

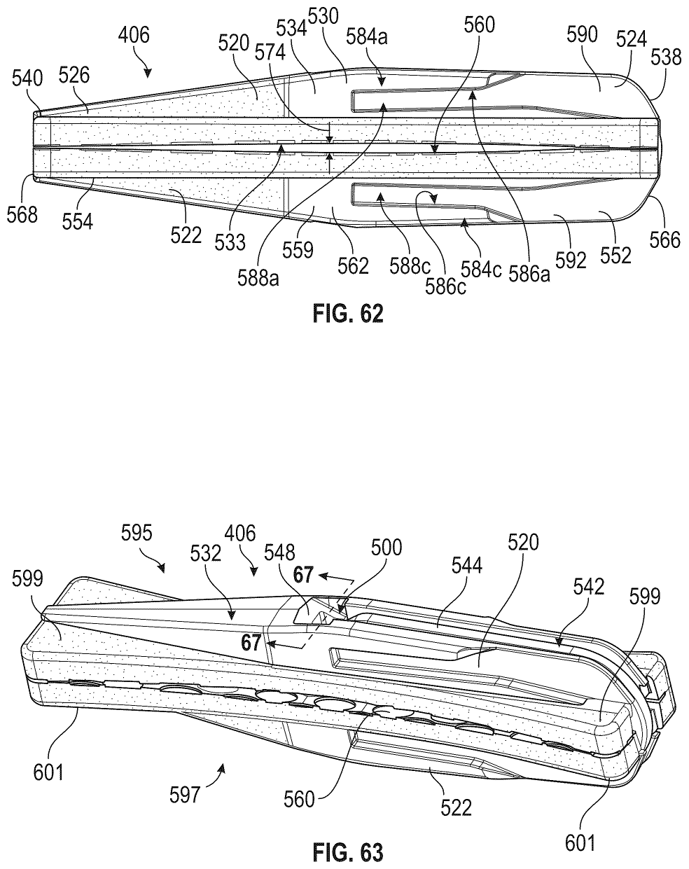

FIG. 62 illustrates a side view of the clip 406. The clip 406 may include the features of the clip 30 unless stated otherwise. The clip 406 may include a first jaw 520 and a second jaw 522. The first jaw 520 may extend from a first end portion 524 to a second end portion 526 along a length of the first jaw 520.

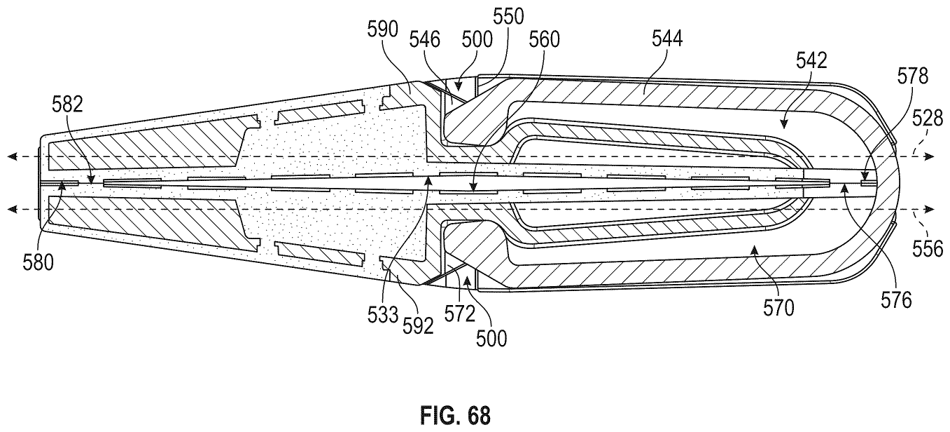

The first jaw 520 may have an elongate shape. The first jaw 520 may be configured as an elongate beam. The first jaw 520 may extend along a longitudinal axis 528 (marked in FIGS. 67 and 68). The first jaw 520 may have a central portion 530 between the first end portion 524 and the second end portion 526.

The first end portion 524 may comprise a proximal end portion of the clip 406. The second end portion 526 may comprise a distal end portion of the clip 406.

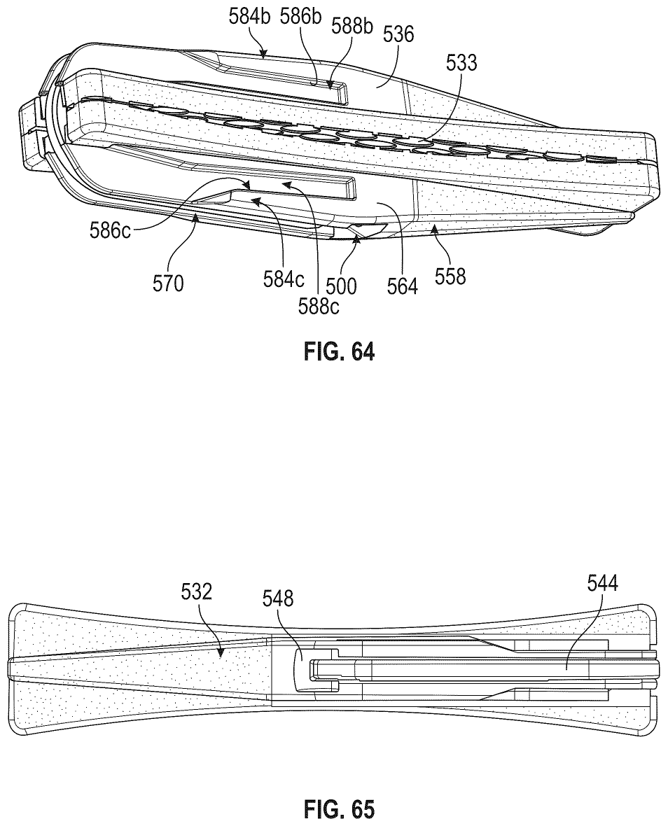

The first jaw 520 may include an outer surface 532 (marked in FIG. 63), a compression surface 533 (marked in FIGS. 62 and 64), and two side surfaces 534, 536 (with side surface 536 shown in FIG. 64) each extending from the compression surface 533 to the outer surface 532. The two side surfaces 534, 536 may face opposite each other. The compression surface 533 may face opposite the outer surface 532 and towards the second jaw 522.

The first jaw 520 may include a first end surface 538 positioned at the first end portion 524 of the first jaw 520. The first jaw 520 may include a second end surface 540 positioned at the second end portion 526 of the first jaw 520. The first end surface 538 may comprise a proximal end surface of the first jaw 520 and the second end surface 540 may comprise a distal end surface of the first jaw 520.

One or more of the side surfaces 534, 536 may be curved in a similar manner as with the side surfaces 44, 46 of the clip 30. For example, the curvature may be a concave curvature relative to the first jaw 520.

The first jaw 520 may include a channel 542 that may be configured similarly as the channel 50 of the clip 30. The channel 542 may be shaped such that the spring 544 may be entirely enclosed on its sides by the body of the first jaw 520. The first jaw 520 may include a coupler 546 (marked in FIG. 68) that may be configured similarly as the coupler 66 of the clip 30.

The outer surface 532 of the first jaw 520 may include the recess 500 for receiving the protrusion 412 of the retention mechanism. The recess 500 may be shaped to receive the protrusion 412 and may include a tapered surface 548 for engaging the corresponding tapered surface 504 of the protrusion 412, and may include a linear or vertical surface 550 for engaging a corresponding linear or vertical surface of the protrusion 412.

The second jaw 522 may be configured similarly as the first jaw 520. The second jaw 522 may extend from a first end portion 552 to a second end portion 554 along a length of the second jaw 522.

The second jaw 522 may have an elongate shape. The second jaw 522 may be configured as an elongate beam. The second jaw 522 may extend along a longitudinal axis 556 (marked in FIGS. 67 and 68). The second jaw 522 may have a central portion 559 between the first end portion 552 and the second end portion 554.

The first end portion 552 may comprise a proximal end portion of the clip 406. The second end portion 554 may comprise a distal end portion of the clip 406.

The second jaw 522 may include an outer surface 558 (marked in FIG. 64), a compression surface 560 (marked in FIGS. 62 and 64), and two side surfaces 562, 564 (with side surface 564 shown in FIG. 64) each extending from the compression surface 560 to the outer surface 558. The two side surfaces 562, 564 may face opposite each other. The compression surface 560 may face opposite the outer surface 558 and towards the first jaw 520.

The second jaw 522 may include a first end surface 566 positioned at the first end portion 552 of the second jaw 522. The second jaw 522 may include a second end surface 568 positioned at the second end portion 554 of the second jaw 522. The first end surface 566 may comprise a proximal end surface of the second jaw 522 and the second end surface 568 may comprise a distal end surface of the second jaw 522.

One or more of the side surfaces 562, 564 may be curved in a similar manner as with the side surfaces 534, 536. For example, the curvature may be a concave curvature relative to the second jaw 522.

The second jaw 522 may include a channel 570 that may be configured similarly as the channel 90 of the clip 30. The channel 570 may be shaped such that the spring 544 may be entirely enclosed on its sides by the body of the second jaw 522. The second jaw 522 may include a coupler 572 (marked in FIG. 68) that may be configured similarly as the coupler 106 of the clip 30.

The outer surface 558 of the second jaw 522 may include a recess 500 for receiving the protrusion 412 of the retention mechanism. The recess 500 may be positioned on the second jaw 522 such that the clip 406 may be engaged by the engagement portion 398 of the expansion apparatus 390 with either the first jaw 520 or the second jaw 522 engaged by the protrusion 412. Inverted positions of coupling with the clip 406 may be utilized.

Referring to FIG. 62, in examples, the compression surface 533 of the first jaw 520 may have a concave curvature relative to the first jaw 520. The compression surface 560 of the second jaw 522 may have a concave curvature relative to the second jaw 522. As such, the compression surface 533 of the first jaw 520 is spaced from the compression surface 560 of the second jaw 522 with a gap 574 when the clip 406 is in a closed configuration. The gap 574 may have an oval shape in examples, although other shapes may be utilized.

In examples, a first end portion 576 (marked in FIG. 68) or proximal end portion of the compression surface 533 may contact a first end portion 578 (marked in FIG. 68) or proximal end portion of the compression surface 560 when the clip 406 is in the closed configuration. A second end portion 580 (marked in FIG. 68) or distal end portion of the compression surface 533 may contact a second end portion 582 (marked in FIG. 68) or distal end portion of the compression surface 560 when the clip 406 is in the closed configuration. The size of the gap 574 between the compression surfaces 533, 560 accordingly may be the greatest at the respective central portions 530, 559 of the jaws 520, 522 and may reduce in directions towards the respective end portions 524, 552, 526, 554 of the jaws 520, 522. The reduction in the size of the gap 574 may vary based on the curvatures of the compression surfaces 533, 560.

The curvatures of the compression surfaces 533, 560 may provide a more even distribution of force upon the tissue being occluded (e.g., tissue of a left atrial appendage (LAA)) than a configuration of a clip having flat or linear compression surfaces. Other forms of compression surfaces may be utilized in examples.

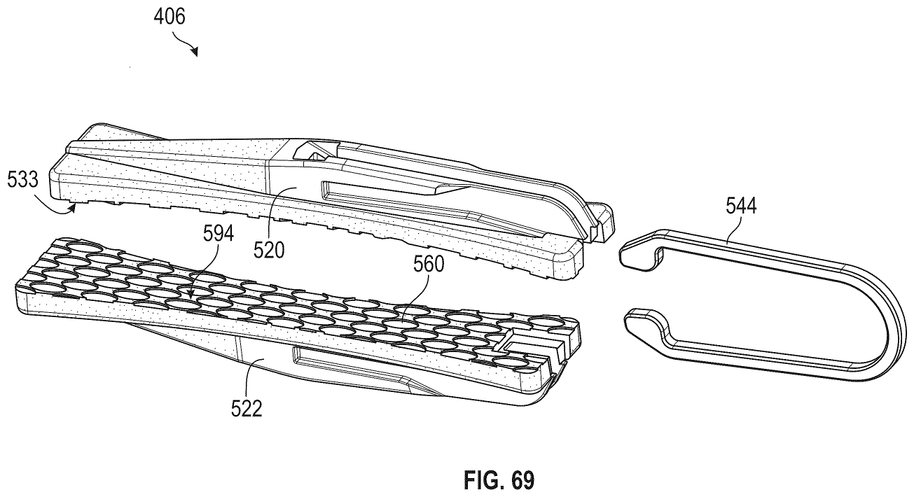

The spring 544 (marked in FIGS. 68 and 69) may include the features of the spring 60. The spring 544 may produce a U-shaped fulcrum. The distal end tips of the clip 406 may have a wide opening to capture the tissue to be occluded. A tip first closure of the clip 406 may result (with the distal end tips of the clip 406 closing prior to the proximal end portions of the clip 406).

Referring to FIGS. 62 and 64, the clip 406 may include one or more elongate couplers 584a-d that may be configured similarly as the one or more elongate couplers 120a-d of the clip 30 unless stated otherwise. The one or more elongate couplers 584a-d may include respective protrusions or rails having inner surfaces 586a-d that angle towards the compression channel between the first jaw 520 and the second jaw 522 along a distal direction of the clip 406. The corresponding channels 588a-d bound by the elongate couplers 584a-d may be angled towards the compression channel between the first jaw 520 and the second jaw 522 along a distal direction of the clip 406. Such a feature may allow for the channels 588a-d to extend parallel with each other as the clip 406 is opened by the arms 408, 410 of the engagement portion 398 of the expansion apparatus 390 (due to the increased angle at the second end portions 526, 554 of the jaws 520, 522 than the first end portions 524, 552 in an opened configuration). The clip 406 accordingly may more easily slide off of the engagement portion 398 to release from the engagement portion 398 upon deployment. Other configurations of elongate couplers may be utilized in examples.

In examples, one or more of the compression surfaces 533, 560 may comprise an atraumatic material. The atraumatic material, for example, may form a covering over a base 590, 592 of the respective first jaw 520 and second jaw 522. The base 590, 592 may comprise an underlying surface that the atraumatic material is applied to. The atraumatic material, for example, may be coated upon the base 590, 592.

In examples, the atraumatic material may be applied by being overmolded upon the base 590, 592. A material may be overmolded with the base 590, 592 inserted into a respective mold for the material. In examples, other forms of coverings or coatings may be provided.

In examples, the atraumatic material may comprise a medical grade soft material. The atraumatic material may comprise an elastomeric material, which may comprise silicone in examples. The silicone may be overmolded upon a respective base 590, 592 and may comprise one or more of the compression surface 533 of the first jaw 520 or the compression surface 560 of the second jaw 522. The atraumatic material or silicone may form all or a portion of the side surfaces 534, 536, 562, 564 of the jaws 520, 522 in examples. In examples, the atraumatic material or silicone may form all or a portion of the outer surfaces 532, 558 of the jaws 520, 522 in examples. Other forms of atraumatic or elastomeric material may be utilized in examples.

Referring to the cross sectional view of FIG. 68, the base 590, 592 is shown to comprise an underlying material that the respective atraumatic material is overmolded onto, to form the compression surface 533 of the first jaw 520 and the compression surface 560 of the second jaw 522. The bases 590, 592 or underlying surfaces may include gates or channels that the atraumatic material enters into upon molding into the bases 590, 592. The atraumatic material may be molded to form the outer surfaces 532, 558 of the jaws 520, 522 distal of the respective couplers 546, 572. For example, referring to FIG. 63, respective leading portions 595, 597 of the jaws 520, 522 distal of the couplers 546, 572 may be covered with the atraumatic material. The leading portions 595, 597 may extend from the couplers 546, 572 to the respective distal tips of the jaws 520, 522. The outer surfaces 532, 558 of the jaws 520, 522 at these leading portions 595, 597 may be covered with the atraumatic material to provide for smooth entry towards the tissue to be occluded. Further, upper flange portions 599, 601 of the respective jaws 520, 522 may be covered with the atraumatic material in examples. The coverings may be provided during an overmolding process. Other portions of the jaws 520, 522 may be formed with the atraumatic material as desired.

In examples, the bases 590, 592 may be made of a material that is more rigid than the atraumatic material. The atraumatic material may be a relatively pliable material compared to the material of the bases 590, 592. The bases 590, 592 may be made of a plastic material such as polyether ether ketone (PEEK) or other forms of plastic materials in examples. The bases 590, 592 may be made from other polymers or materials such as metals, alloys, or other forms of materials.

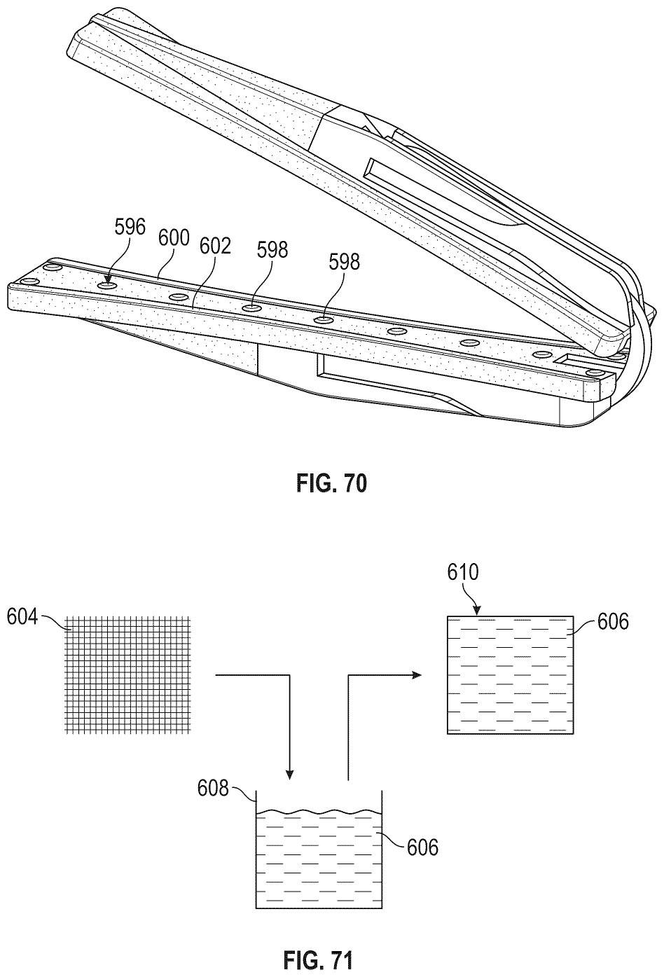

In examples, the atraumatic material may be textured to enhance grip to the tissue being occluded. FIG. 69, for example, illustrates an assembly view of the clip 406 illustrating an exemplary textured surface 594 of the second jaw 522 comprising the compression surface 560. The textured surface 594 may comprise a pattern of recesses in the compression surface 560. The first jaw 520 may have a similar textured surface on the compression surface 533. Variations in the configuration of the textured surface may be provided. FIG. 70, for example, illustrates a variation in which the textured surface 596 includes a series of bumps 598 bounded by elongate rails 600, 602.

Other forms of atraumatic materials may be utilized. FIG. 71, for example, illustrates an exemplary process of forming a material that may be utilized. The material may comprise a fabric 604 that may be coated in a material such as an elastomeric material to form a coated material 610. The elastomeric material, for example, may comprise silicone 606 (represented in the bath 608). The fabric 604 may be dipped into the bath 608 to coat the fabric 604 with silicone 606. The silicone 606 may be dip coated upon the fabric 604. The fabric 604 may be removed from the bath 608 to form the coated material 610 comprising the fabric 604 with the silicone 606 coated thereon. The material 610 may be stretched and hung vertically in examples. The material 610 may be maintained at a desired temperature (e.g., room temperature or a higher temperature in an oven) to allow the material 610 to dry and any solvent to dissipate. In examples, an antimicrobial coating may be positioned upon the material 610 as desired. The material 610 may then be cut and applied upon the jaws 520, 522 as desired (e.g., to form the compression surface 533 of the first jaw 520 and the compression surface 560 of the second jaw 522). Other materials may be coated upon the fabric 604 as desired.

Other methods of producing a coated fabric may be utilized. For example, the fibers that form a fabric may be coated in an atraumatic or elastomeric material (e.g., such as silicone). The coated fibers may then be woven or otherwise formed into the fabric. The atraumatic or elastomeric material (e.g., such as silicone) may remain coated upon the fibers forming the resulting fabric. A fabric tube or other structure may be formed. The resulting fabric may then be cut and applied upon the jaws 520, 522 as desired (e.g., to form the compression surface 533 of the first jaw 520 and the compression surface 560 of the second jaw 522). The coated fibers may result in a texture for the compression surface to which it is applied. Other methods may be utilized in examples.

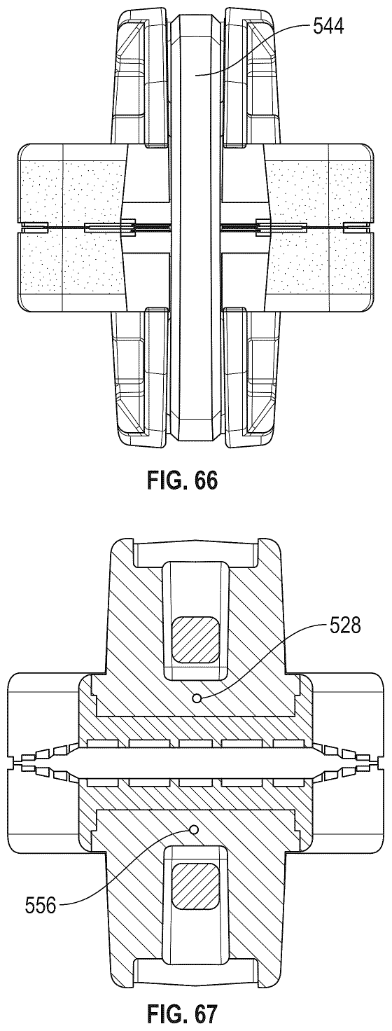

The features of FIGS. 62-71 may be utilized solely or in combination with any other example herein.

Various other configurations of clips may be utilized in examples.

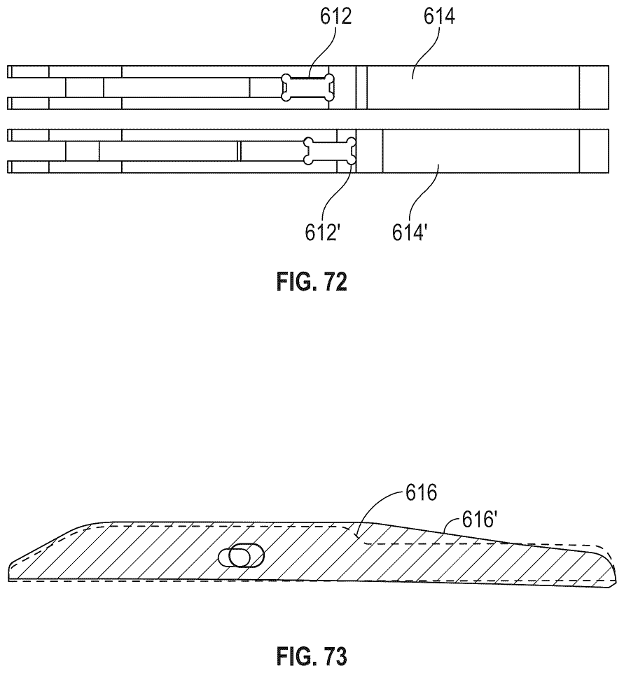

In examples, the configurations of the clips disclosed herein may be modified to produce a more uniform distribution of force or pressure distribution to the tissue being occluded from the proximal end portions of the jaws to the distal end portions of the jaws. A more uniform distribution of force or pressure distribution may reduce the possibility of leaks from the occluded tissue and damage to the tissue. Features that may be modified, for example, may comprise a position of the coupler (e.g., a coupler 66 or coupler 106 as shown in FIG. 2G). A coupler 612 as represented in FIG. 72 for a jaw 614, for example, may be moved to a position (represented by coupler 612′) that is more distal than a center point of the jaw 614, to more evenly distribute the force from the spring (not shown in FIG. 72) to the jaw 614′. The more distal positioning of the coupler 612′ may be desirable because the spring for the clip may exert more force in a proximal portion of the clip. As such, a more distal position of the coupler 612′ may more evenly distribute the force from the spring for the clip. A more uniform distribution of force may more evenly occlude the underlying tissue compressed by the clip.

Other features that may be modified include the distribution of material forming the respective jaws. FIG. 73, for example, illustrates an exemplary jaw 616. The material forming the jaw 616 may be more evenly distributed to the distal end portion of the jaw 616 to form a profile of the jaw 616′. A more even distribution of material may result, and a more even force distribution may result. Other features that may be varied include an increased spring width, which may provide a greater force that is more evenly applied by the clip.

The features of FIGS. 72 and 73 may be utilized solely or in combination with any other example herein.

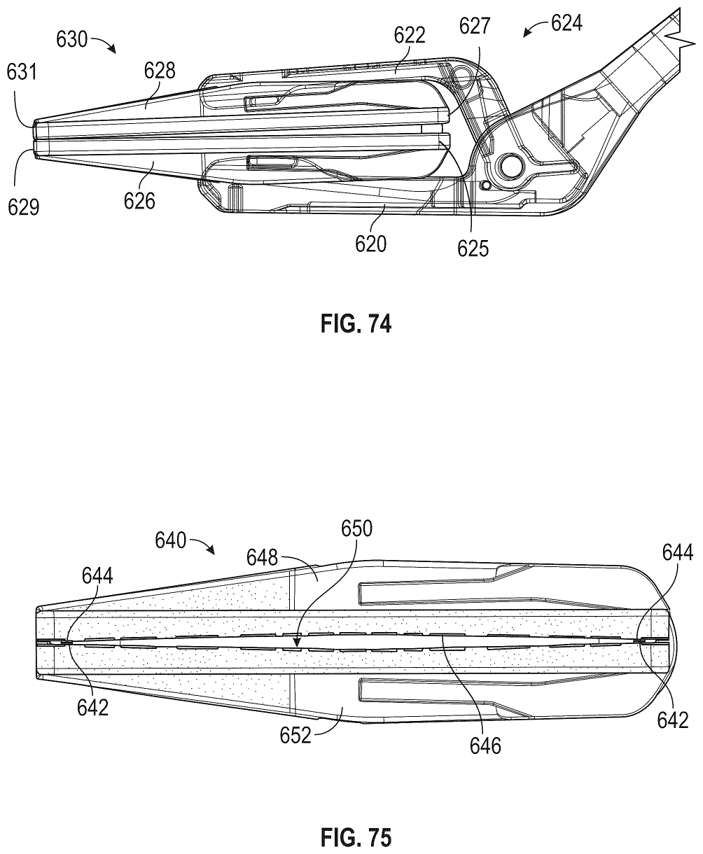

Variations in configurations of expansion apparatuses disclosed herein may be utilized. FIG. 74, for example, illustrates a variation in which respective arms 620, 622 of an engagement portion 624 retain the jaws 626, 628 of the clip 630 at an angle for additional securement to the engagement portion 624. The clip 630 as shown in FIG. 74 is secured to the engagement portion 624, with the proximal ends 625, 627 of the jaws 626, 628 spaced from each other due to the angle of the jaws 626, 628, and the distal ends 629, 631 of the jaws 626, 628 in contact with each other. A friction force or interference fit may be provided between the arms 620, 622 and the jaws 626, 628 to retain the clip 630 to the engagement portion 624. The friction force may be between the elongate couplers of the clip 630 and the rails of the arms 620, 622. The clip 630 may be configured similarly as the clip 406 unless stated otherwise. The engagement portion 624 may include the features of the engagement portion 398 unless stated otherwise. At a desired time of deployment, an occlusive force applied to the clip 630 may clear the jaws 626, 628 of the clip 630 from rails of the engagement portion 624 in a similar manner as represented in FIG. 45.

In examples, another form of retention mechanism may be utilized in combination, with the retention mechanism comprising a protrusion 412 that is actuatable via a tether 414 (as shown in FIG. 60). Other forms of retention mechanisms may be utilized in examples.

The features of FIG. 74 may be utilized solely or in combination with any other example herein.

Clips disclosed herein may include compression surfaces that may potentially bond to each other if left in contact with each other. For example, materials that may form the compression surfaces may cross-link or otherwise bond with each other if maintained in physical contact and with pressure between each other for extended periods. Silicone compression surfaces as disclosed herein may potentially bond with each other. Such a result is undesirable because storage of such clips, whether coupled or uncoupled to an expansion apparatus, may result in bonding of the compression surfaces over time. Spacer devices accordingly may be desirable to space the compression surfaces from each other such that the compression surfaces are retained separated from each other. A reduced possibility of cross-linking or other forms of bonding between surfaces of the clips may result.

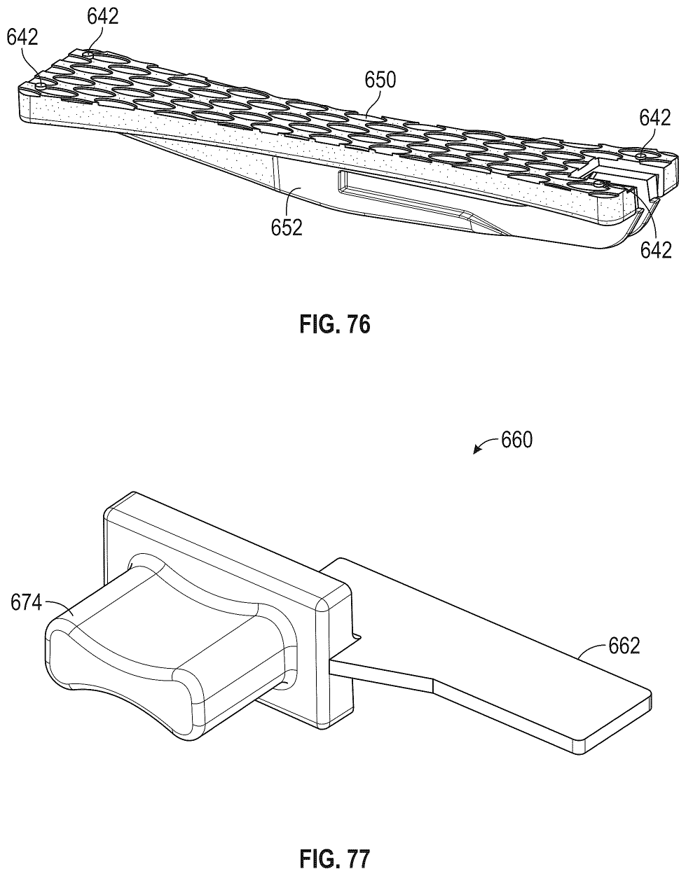

FIG. 75, for example, illustrates a clip 640 having one or more spacer devices 642, 644 for spacing a compression surface 646 of a first jaw 648 from a compression surface 650 of a second jaw 652 such that the compression surfaces 646, 650 are retained separated from each other. The clip 640 may be configured similarly as the clip 406 unless stated otherwise. Referring to FIG. 76, the spacer devices 642 for example, may comprise protrusions on the compression surface 650. The compression surface 650 may comprise an elastomeric material (e.g., silicone) and the spacer devices 642 may comprise protrusions raised above the compression surface 650 and uncovered by the elastomeric material. The spacer devices 642 may comprise the material forming a base of the second jaw 652 that the elastomeric material is formed upon (e.g., overmolded upon). The material of the spacer devices 642, for example, may have a reduced possibility of cross-linking or otherwise bonding with the compression surface 646 of the first jaw 648 (e.g., the spacer devices may be made of PEEK or another form of plastic material or metals or alloys, among other materials).

In examples, the first jaw 648 may include the spacer devices 644 that are configured similarly as the spacer devices 642. The spacer devices 644 may protrude from the compression surface 646 and may align in position with the spacer devices 642 of the second jaw 652. As such, upon closure of the first jaw 648 and the second jaw 652, the spacer devices 642, 644 may contact each other with a reduced possibility of bonding or cross-linking of the surfaces in contact between the first jaw 648 and second jaw 652. In examples, a single one of the first jaw 648 or the second jaw 652 may include the spacer devices. Other methods of reducing cross linking or bonding may include coating the compression surfaces with a non-stick material such as a lubricant or other forms of non-stick materials.

The spacer devices 642 are illustrated in FIG. 76 as being positioned at the corners of the compression surface 650 (e.g., at the proximal and distal end portions), yet other positions may be utilized as desired. In examples in which the compression surfaces are curved concave relative to the respective jaw, the position at the corners may allow the contacting end portions of the compression surfaces to include the spacer devices 642, 644.

Variations in the configuration of a spacer device may be provided in examples. FIGS. 77-125 illustrate forms of spacer devices that may be removed prior to deployment of a clip. The spacer devices represented in FIGS. 77-125 are configured to space a compression surface of a first jaw of a clip from a second compression surface of a second jaw of the clip such that the first compression surface and the second compression surface are retained separated from each other.

FIG. 77, for example, illustrates a spacer device 660. The spacer device 660 may include an insertion body 662 that may be configured to be positioned between a first compression surface 664 of a first jaw 666 of a clip 668 (marked in FIG. 79) and a second compression surface 670 of a second jaw 672 of the clip 668 (marked in FIG. 79) to space the first compression surface 664 from the second compression surface 670.

The insertion body 662 may comprise a planar surface configured to extend parallel with the compression surfaces 664, 670. The insertion body 662 may be configured to contact the compression surfaces 664, 670 upon being inserted between the compression surfaces 664, 670. The insertion body 662 is preferably made of a material that does not cross-link or otherwise bond with the materials comprising the compression surfaces 664, 670 (e.g., a PEEK or plastic material, or metal material, among other forms of materials). Other forms of spacer devices disclosed herein may be made of similar materials.

The spacer device 660 may include a removal body 674 for removing the spacer device 660, thereby removing the spacing of the first compression surface 664 from the second compression surface 670. The removal body 674, for example, may comprise a grip surface that is configured to be gripped by a user to remove the spacer device 660. The removal body 674 may be positioned to a side of the insertion body 662 such that the removal body 674 may be accessed by a user when the insertion body 662 is positioned between the compression surfaces 664, 670. The insertion body 662 may extend from the removal body 674 in a lateral dimension. The removal body 674 may be positioned such that removal occurs in a direction transverse to a longitudinal dimension of the clip 668.

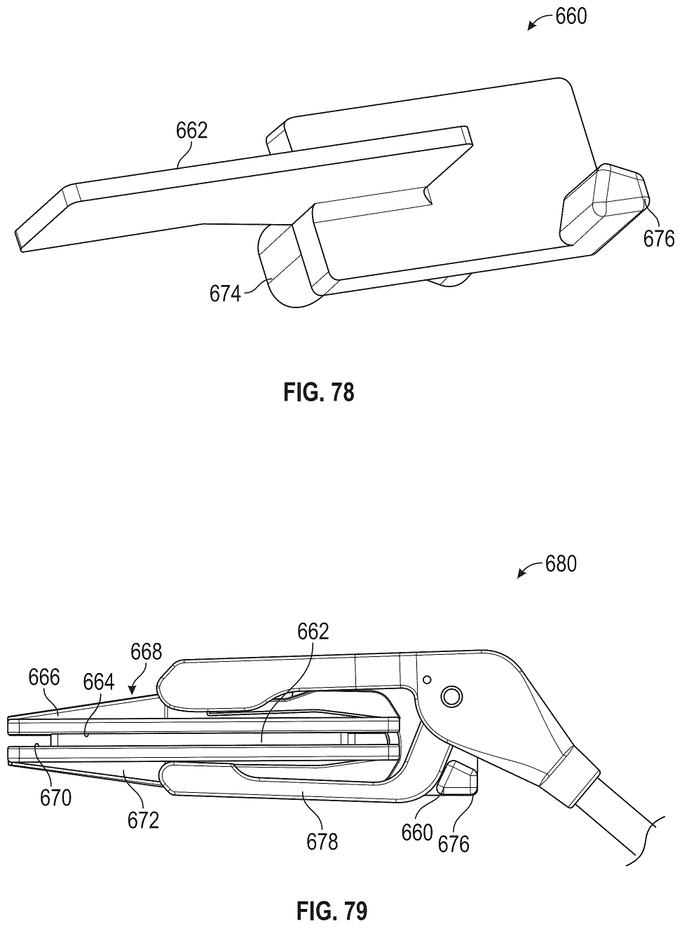

FIG. 78 illustrates a perspective view of the spacer device 660 at an opposite view than shown in FIG. 77. The spacer device 660 includes a stopper body 676 protruding in a lateral dimension from the removal body 674. The stopper body 676 protrudes from the removal body 674 in the lateral dimension in a similar lateral direction that the insertion body 662 protrudes from the removal body 674 in. The stopper body 676 is positioned to abut a portion of the engagement portion of the expansion apparatus. The abutment between the stopper body 676 and the engagement portion reduces the possibility of undesired removal of the spacer device 660 in a longitudinal direction relative to the clip 668.

FIG. 79, for example, illustrates the spacer device 660 in position and engaged with the clip 668. The stopper body 676 protrudes behind an arm 678 of the engagement portion 680 to impede undesired removal of the spacer device 660 in a longitudinal direction relative to the clip 668.

The insertion body 662 remains positioned between the compression surfaces 664, 670 to maintain the spacing between the compression surfaces 664, 670. The clip 668 may be configured similarly as the clip 406 or any other form of clip disclosed herein. The clip 668 may include flattened compression surfaces 664, 670 or curved compression surfaces as disclosed in regard to the clip 406. The engagement portion 680 may be configured similarly as any form of engagement portion disclosed herein, such as the engagement portion 398.

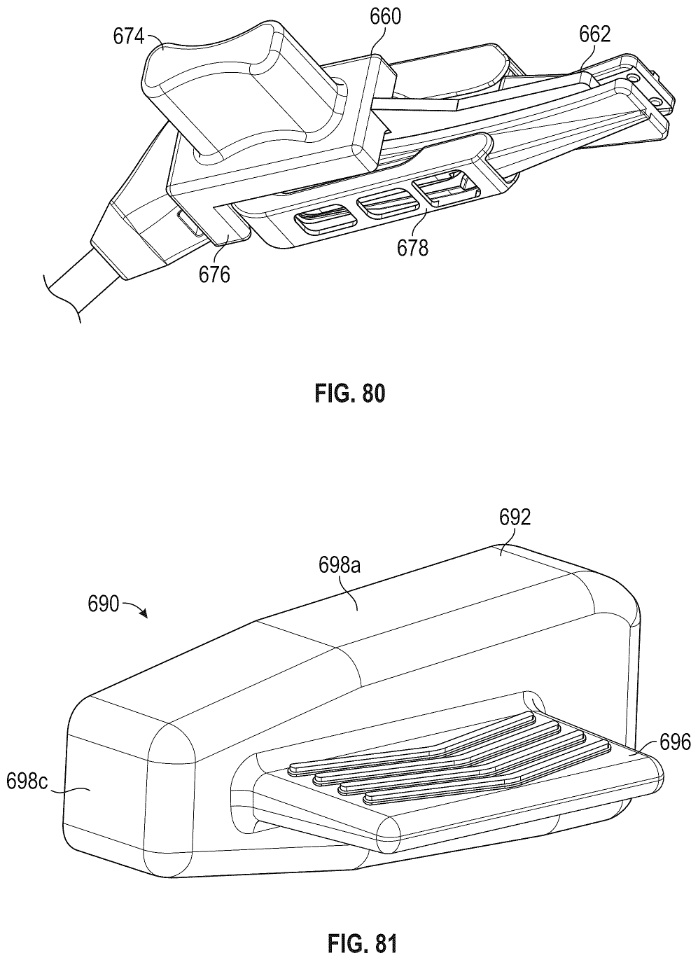

FIG. 80 illustrates a perspective view of the spacer device 660 in position.

The spacer device 660 may remain in position for an extended period of time, which may include a duration of storage of the clip 668 prior to a deployment procedure. The spacer device 660 may remain coupled to the clip 668 for a storage period of the clip 668. The spacer device 660 may similarly be engaged with the clip 668 that is engaged with the engagement portion 680 of the expansion apparatus (as represented in FIGS. 79 and 80). The system may be stored in such a configuration until an intended time of use. Other spacer devices disclosed herein may be utilized in a similar manner unless stated otherwise.

At an intended time of use, the spacer device 660 may be removed via a user pulling the removal body 674 to thus remove the spacer device 660 from between the compression surfaces 664, 670. The clip 668 may be deployed in an intended manner.

Various other configuration of spacer devices may be disclosed herein.

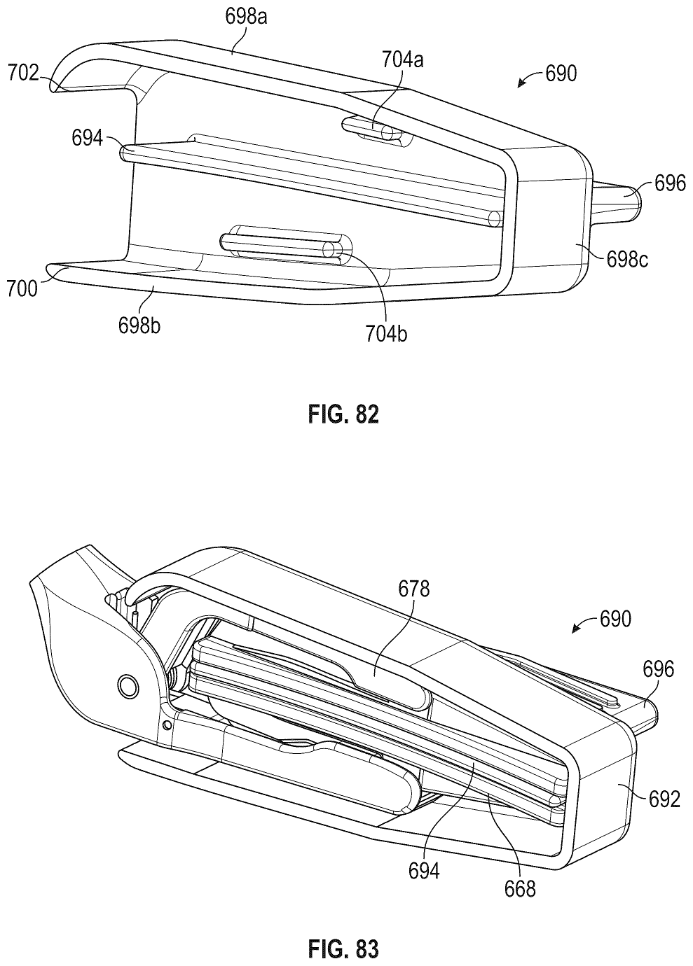

FIGS. 81-83, for example, illustrate a variation in which the spacer device 690 includes a cover 692 for covering at least one exterior surface of a clip. Referring to FIG. 82, the spacer device 690 may include an insertion body 694 that may be configured similarly as the insertion body 662 of the spacer device 660. The insertion body 694 includes a planar surface configured to contact and extend parallel with the first compression surface and the second compression surface of a clip.

Referring to FIG. 81, the spacer device 690 may include a removal body 696 that may be configured similarly as the removal body 674 of the spacer device 660. The removal body 696 is configured to be gripped by a user and used to remove the spacer device 690 from the clip, thereby removing the spacing of the first compression surface from the second compression surface of the clip. The removal body 696 may be positioned such that the spacer device 690 is configured to be removed from the clip in a direction transverse to a longitudinal dimension of the clip.

The cover 692 may include one or more walls that may be configured to cover exterior surfaces of a clip. An upper wall 698a and a lower wall 698b (marked in FIG. 82) may cover respective upper exterior and lower exterior surfaces of the clip. A distal wall 698c may cover a distal end surface of the clip. The cover 692 may include a side opening 700 (marked in FIG. 82) and a proximal opening 702 for the clip and a portion of the engagement portion of the expansion apparatus to be passed through.

Referring to FIG. 82, the spacer device 690 may include bumper bodies 704a, b that may abut respective arms of an expansion apparatus with the spacer device 690 in position. FIG. 83, for example, illustrates the spacer device 690 upon the clip 668. The cover 692 extends over the clip 668 and the engagement portion of the expansion apparatus. The cover 692 may be removed by the removal body 696 being pulled in a direction transverse to the longitudinal dimension of the clip 668. The insertion body 694 may thus be removed.

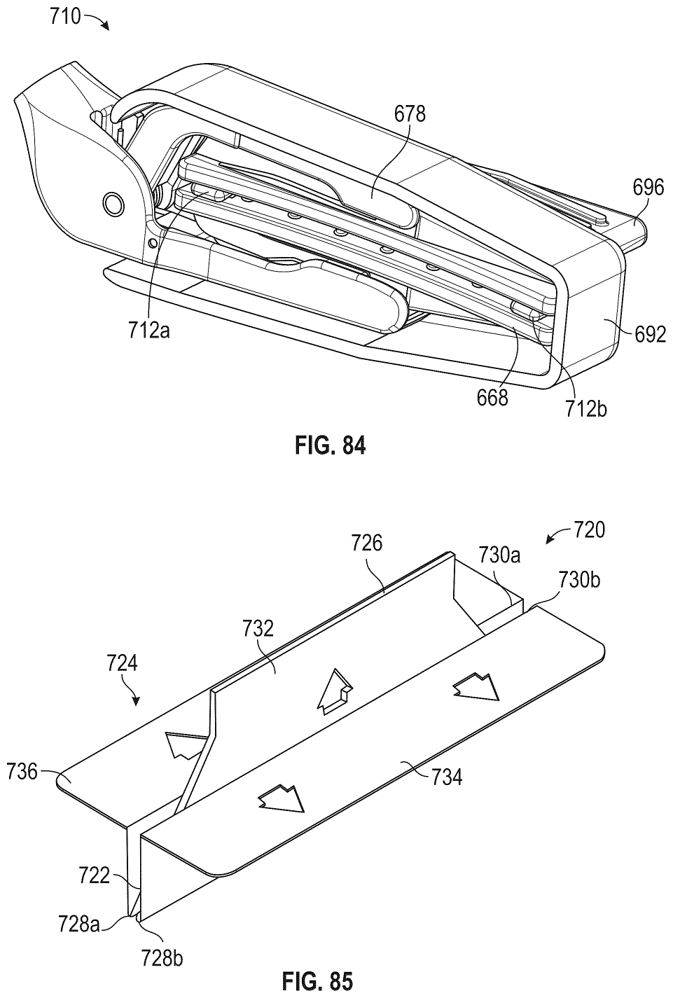

FIG. 84 illustrates a spacer device 710 comprising a variation of the spacer device 690. The spacer device 710 may include a variation of an insertion body comprising multiple spaced prongs 712a, b for insertion between the compression surfaces of the clip 668. The use of multiple spaced prongs 712a, b may reduce a friction force upon removal of the spacer device 710.

Other configurations of spacer devices may be utilized in examples. FIG. 85, for example, illustrates a spacer device 720 having an insertion body 722 comprising a folded substrate. The folded substrate is configured to be positioned between and contact the compression surfaces of a clip to space the surfaces from each other. The folded substrate may comprise a planar surface for extending parallel with compression surfaces of the clip. The spacer device 720 may comprise a single piece of material folded to form the insertion body 722 and a removal body 724 of the spacer device 720. In examples, the substrate may comprise a cut sheet of material such as non-woven fiber such as polyethylene fibers (such as Tyvek), or a film, or non-stick or lubricious polymer.

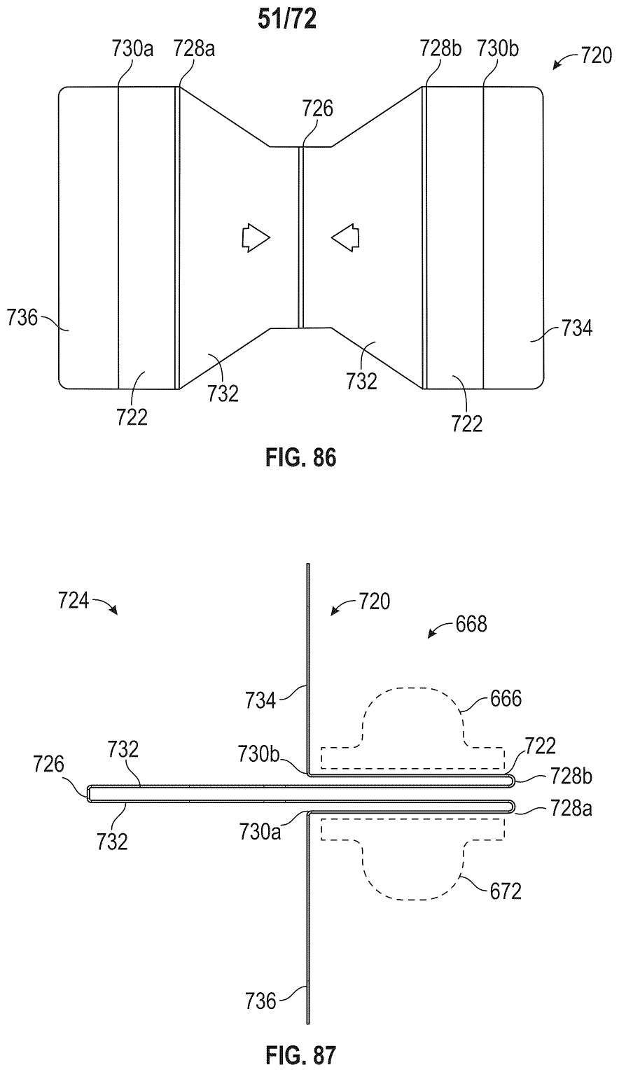

FIG. 86, for example, illustrates the spacer device 720 in a flattened configuration. The spacer device 720 may include fold lines 726, 728a, b, 730a, b that may be utilized to fold the spacer device 720 from the flattened configuration shown in FIG. 86 to the folded configuration shown in FIG. 85. The spacer device 720 as shown in FIG. 86 may be folded along the fold line 726 to form a first portion 732 of the removal body 724. The spacer device 720 may be folded along the fold lines 728a, b to form the folded substrate of the insertion body 722. The remaining end tabs provided outside of the fold lines 730a, b may form a respective second portion 734 and a third portion 736 of the removal body 724. The portions 734, 736 may be configured to be pulled to unfold the substrate of the insertion body 722.

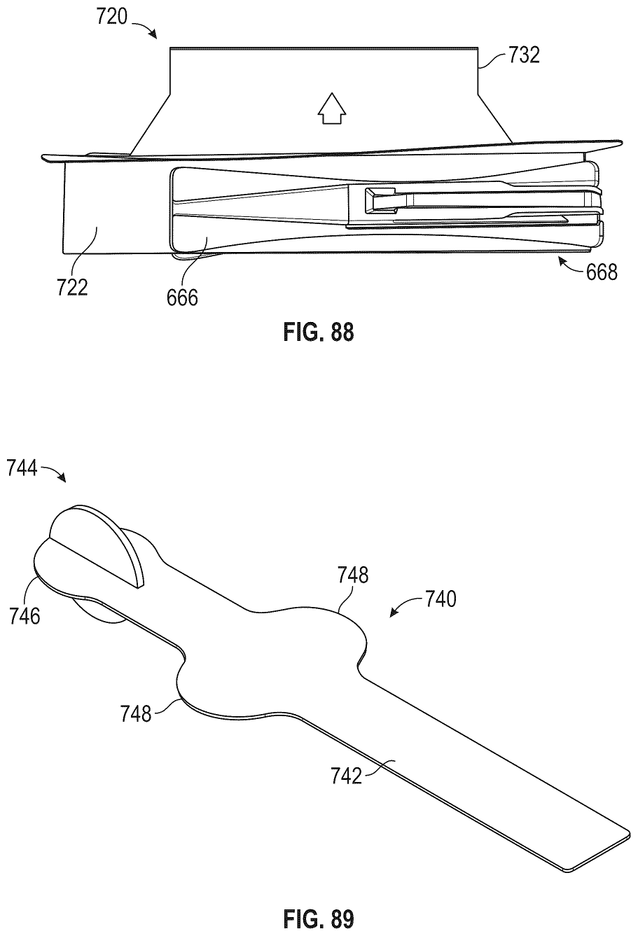

FIG. 87 illustrates a cross sectional view of the folded spacer device 720 positioned between jaws 666, 672 of the clip 668. The portions 734, 736 may form a stopper upon the spacer device 720 being inserted between the jaws 666, 672. FIG. 88 illustrates a perspective view of the folded spacer device 720 positioned between the jaws 666, 672 of the clip 668.

To remove the spacer device 720, one or more of the portions 732, 734, 736 of the removal body 724 may be pulled. Gripping and pulling one or more of the portions 732, 734, 736 may operate to unfold the substrate of the insertion body 722, and thereby removing the spacing of the first compression surface of the clip from the second compression surface of the clip. The spacer device 720 may be removed in a direction transverse to a longitudinal dimension of the clip.

Other configurations of spacer devices may be utilized in examples. FIG. 89, for example, illustrates a variation of a spacer device 740 including an insertion body 742 comprising an elongate strip of material for extending between the compression surfaces of a clip. The spacer device 740 may include a removal body 744 having multiple portions. A portion 746 of the removal body 744 may be configured for a longitudinal or axial pull for removal of the spacer device 740. Portions 748 of the removal body 744 may be configured for a side or lateral pull for removal of the spacer device 740. The portions 746, 748 may each comprise tabs protruding from the insertion body 742. In examples, the material of the insertion body may comprise a high-density polyethylene (HDPE) or other forms of materials. Any of the spacer devices disclosed herein may be formed with a high-density polyethylene (HDPE) material or polyurethane, or other polymer material. Other configurations may be utilized in examples.

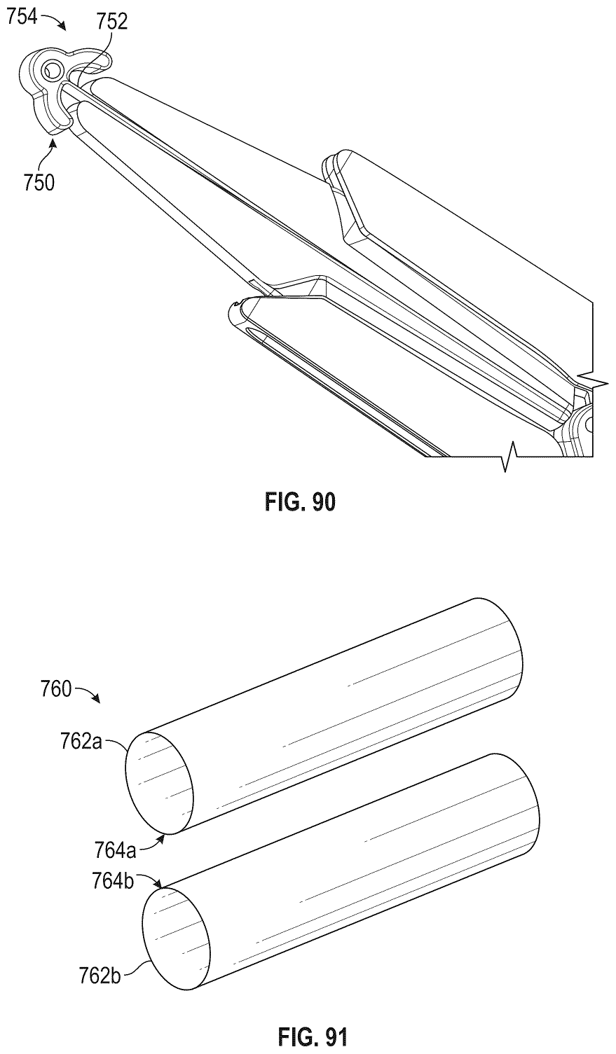

FIG. 90, for example, illustrates a variation of a spacer device 750 having an insertion body 752 comprising an elongate strip of material, similar to the insertion body 742 shown in FIG. 89. The removal body 754 may comprise a molded body (e.g., injection molded) positioned at an end of the insertion body 752 and configured to be pulled longitudinally or axially for removal of the spacer device 750. The removal body 754 may be formed integral with the insertion body 752 in examples.

Other configurations of spacer devices may be utilized in examples. FIG. 91 illustrates an example of a spacer device 760 including one or more sheaths 762a, b. Each sheath 762a, b may have a section forming an insertion body 764a, b for being positioned between the compression surfaces of the jaws of the clip. The respective sheath 762a, b may comprise a cover for the jaw of the clip to which it is applied.

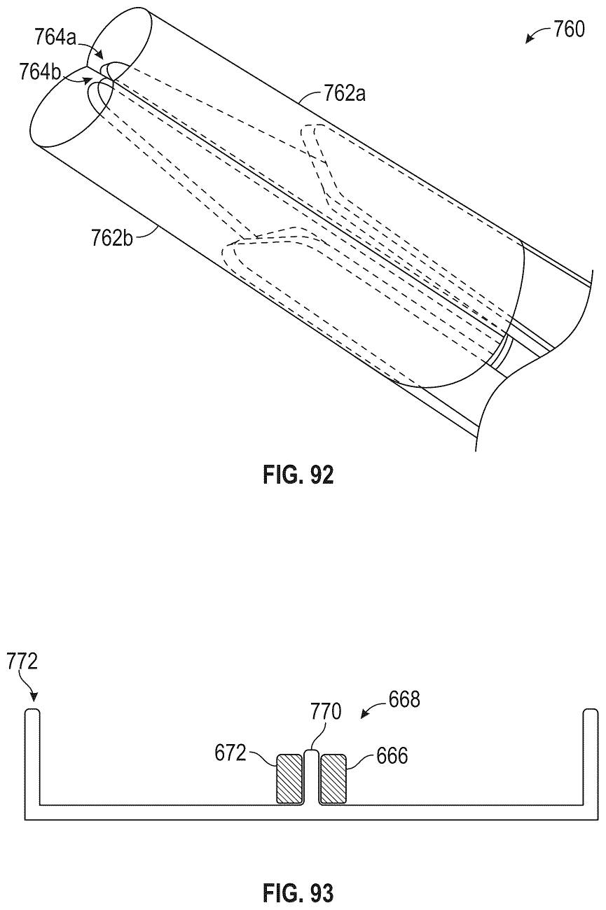

FIG. 92, for example, illustrates the sheaths 762a, b upon the jaws of the clip. The sheaths 762a, b may further cover the arms of the engagement portion of the expansion apparatus. The sheaths 762a, b may be removed prior to a deployment of the clip by being removed along a longitudinal dimension of the clip.

Other configurations of spacer devices may be utilized in examples.

FIG. 93 illustrates a cross sectional representation of a spacer device 770 comprising a portion of packaging 772 for a clip and/or for an expansion apparatus for the clip. The packaging 772 is shown in FIG. 93 to comprise a tray for holding the clip and/or an expansion apparatus, although other forms of packaging may be utilized in examples. The spacer device 770 may be integrated with the packaging 772 such that the compression surfaces of the jaws 666, 672 of the clip are spaced from each other when the clip is retained by the packaging 772.

The spacer device 770, for example, may include an insertion body that inserts between the jaws 666, 672 of the clip. The insertion body may comprise a molded portion of the packaging 772 (e.g., a molded portion of a tray or other form of packaging) or may comprise a separate material (such as a loop or other structure). The insertion body may be removed upon the clip being removed from the packaging 772.

Other configurations of spacer devices may be utilized in examples.

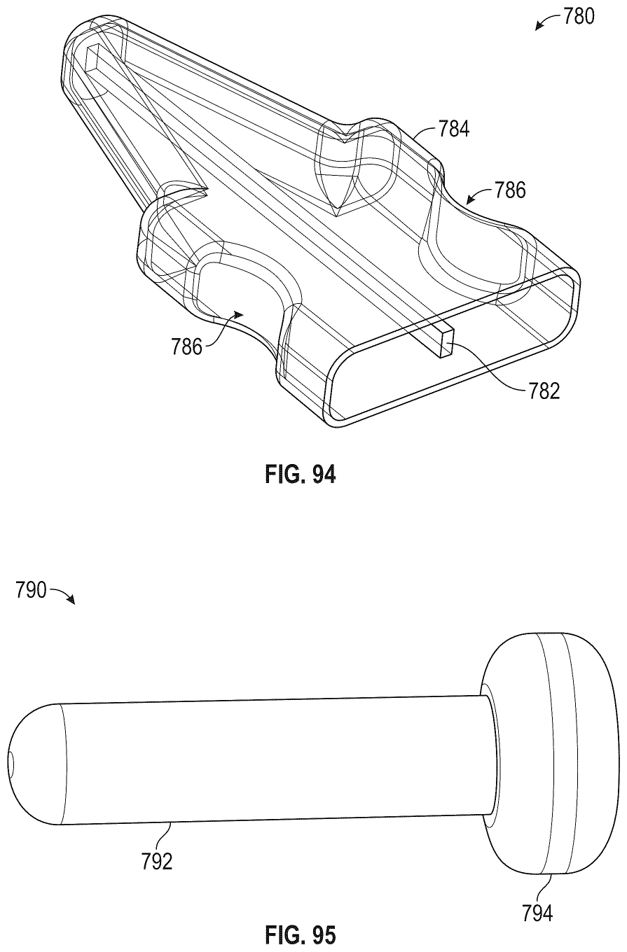

FIG. 94, for example, illustrates a variation of a spacer device 780 including an insertion body 782 covered by a cover 784 comprising a longitudinally or axially extending sheath. The cover 784 may extend longitudinally or axially over a clip and/or engagement portion of an expansion apparatus with the insertion body 782 positioned between the compression surfaces of the clip. The insertion body 782 may comprise a planar surface configured to extend parallel with the compression surfaces of the clip.

The spacer device 780 may further include a removal body 786 in the form of indentations on the cover 784 configured for grip and axial removal of the cover 784 from the clip and engagement portion. The spacer device 780 may be removed from the clip along a longitudinal dimension of the clip. The insertion body 782 and cover 784 may comprise a single molded body in examples (e.g., injection molded), although other configurations may be utilized in examples.

Other configurations of spacer devices may be utilized in examples. In examples, a spacer device may space the compression surfaces of a clip by engaging a portion of the expansion apparatus, for example, the engagement portion of an expansion apparatus. FIGS. 95-125 illustrate examples of spacer devices for engaging an engagement portion of an expansion apparatus to space the compression surfaces of a clip.

FIG. 95, for example, illustrates a spacer device 790 including a pin 792 for engaging an engagement portion of an expansion apparatus. The spacer device 790 may include a removal body 794 for removing the spacer device 790 and thus removing the spacing of the compression surfaces of the clip from each other. The removal body 794 may comprise an enlarged head of the spacer device 790 coupled to the pin 792. The removal body 794 may be configured to be gripped by a user to remove the spacer device 790.

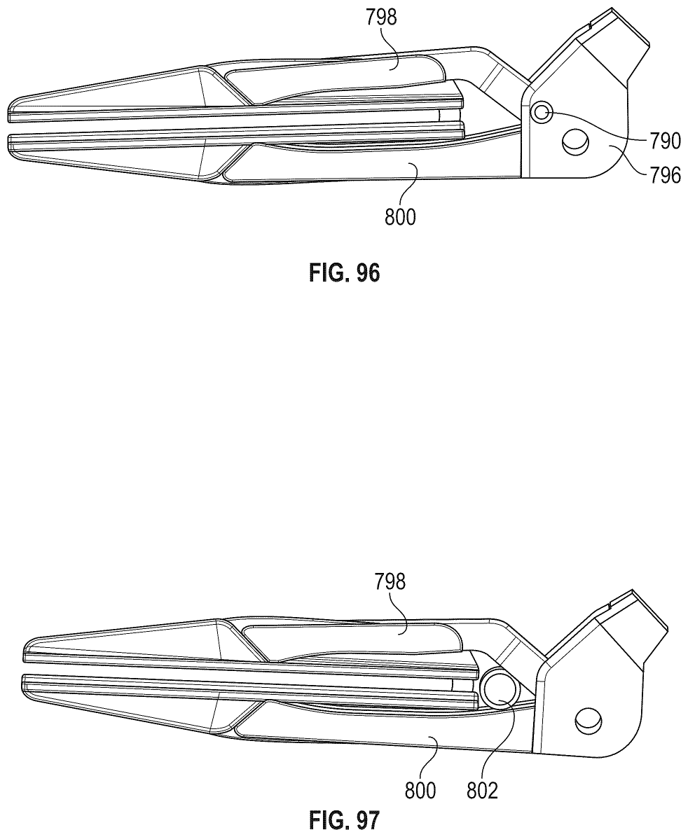

FIG. 96 illustrates an exemplary use of the spacer device 790. The spacer device 790 may insert into an opening or aperture that extends through the base 796 and through the second arm 798. The pin 792 may resist a compressive force between the arms 798, 800 of the engagement portion to retain a separation between the compressive surfaces of the clip.

The pin 792 may be removed at a desired time by a user gripping the removal body 794 and pulling the pin 792 from the opening or aperture.

Other configurations and positions of a pin may be utilized. FIG. 97, for example, illustrates a variation in which a spacer device 802 comprises a larger pin than shown in FIG. 95 and is configured to be wedged between the arms 798, 800 of the engagement portion and directly contact the arms 798, 800.

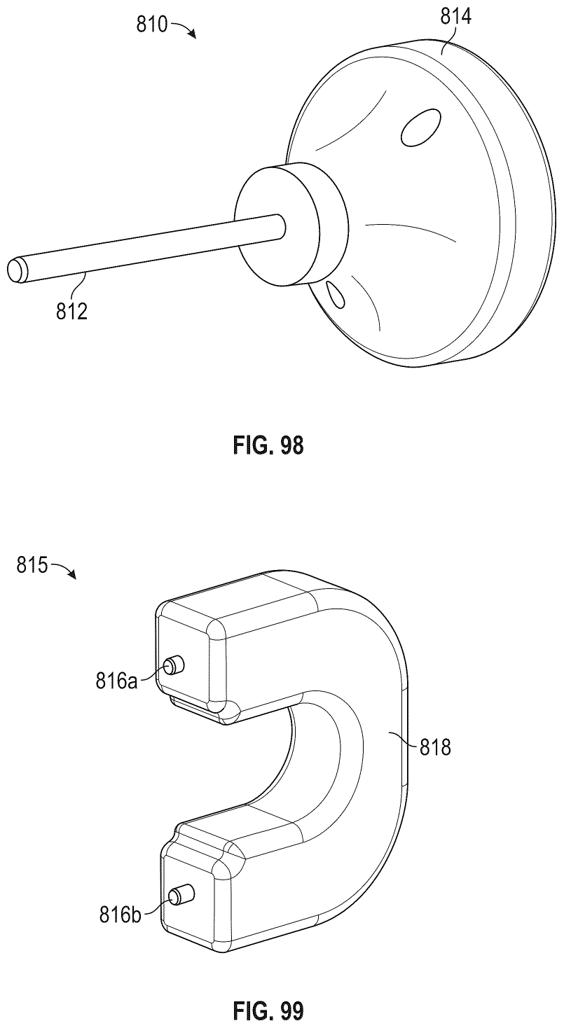

Other configurations of pins may be utilized in examples. FIG. 98, for example, illustrates a variation of a spacer device 810 including a pin 812 and a removal body 814 in the form of a knob. The spacer device 810 may engage the engagement portion of the expansion apparatus in a similar manner as the spacer devices 790, 802.

Other configurations of pins may be utilized in examples. FIG. 99, for example, illustrates a variation of a spacer device 815 in which two support bodies in the form of pins 816a, b may be utilized that are connected by the removal body 818. The removal body 818 may have a “U” shape or other shape as desired. A first pin 816a may be configured to engage a first arm of the engagement portion. A second pin 816b may be configured to engage a second arm of the engagement portion. The removal body 818 may extend between and space the pins 816a, b from each other. The removal body 818 may have sufficient strength to resist a compressive force between the arms of the engagement portion. The removal body 818 is configured to be gripped and removed to remove the spacer device 815.

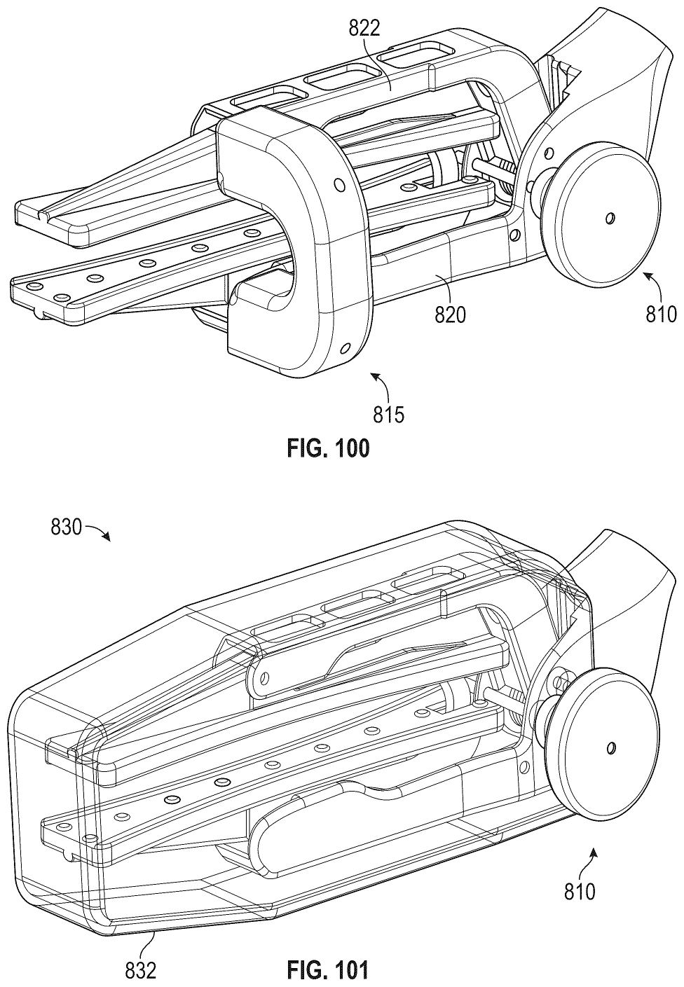

FIG. 100 illustrates a deployment of the spacer device 815. The respective arms 820, 822 of the engagement portion are engaged by the spacer device 815. The arms 820, 822 of the engagement portion are separated by the spacer device 815, with the spacer device 815 resisting a compression force between the arms 820, 822. The spacer device 815 is removable in a direction transverse to the longitudinal dimension of the clip. The spacer device 810 of FIG. 98 is shown deployed as well. The spacer devices 810, 815 may be utilized solely or in combination with each other in examples.

Other configurations of spacer devices may be utilized in examples. FIG. 101, for example, illustrates a spacer device 830 comprising a combination of the spacer device 810 and a cover 832 for the clip and portion of the engagement portion of the expansion apparatus. The cover 832 may be configured similarly as the cover 692 shown in FIG. 81, yet the spacer device 810 may extend through an aperture in the cover 832 to engage the engagement portion of the expansion apparatus. The spacer device 810 may operate in a similar manner as shown in FIG. 100. The cover 832 may be held to the expansion apparatus via the spacer device 810 and may be removed by a user pulling the spacer device 810 from the expansion apparatus.

Other forms of spacer devices may be utilized in examples.

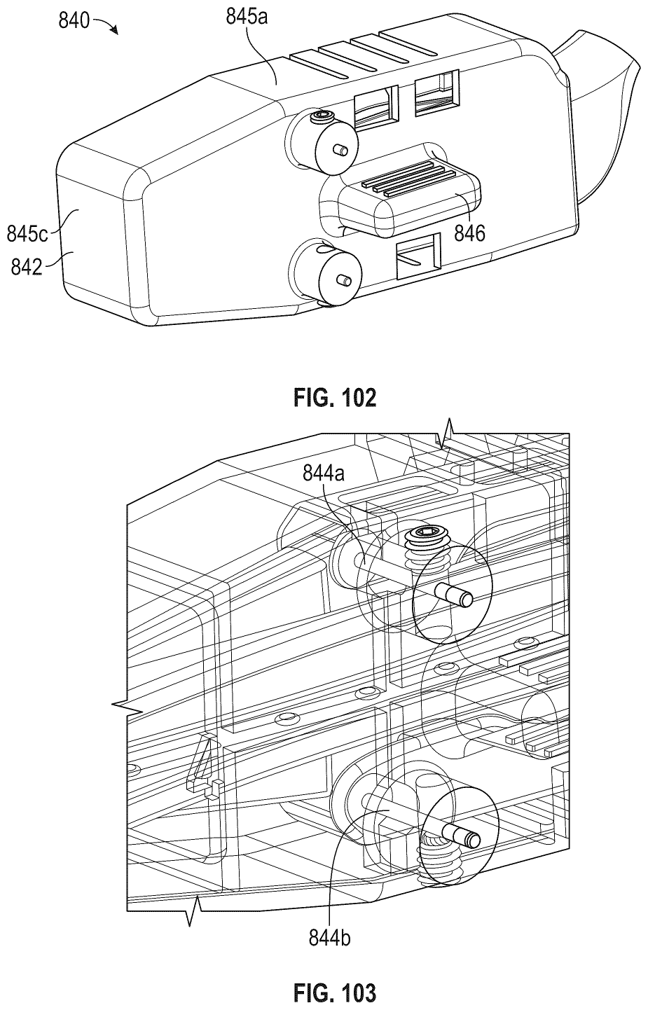

FIGS. 102-104, for example, illustrate a spacer device 840 comprising a combination of a cover 842 and a plurality of support bodies in the form of pins 844a, b (shown in the transparency view of FIG. 103). The pins 844a, b may extend from the cover 842.

The cover 842 may be configured similarly as the cover 692 shown in FIG. 81 and may include one or more walls 845a-c that may cover surfaces in a similar manner as the respective walls 698a-c of the cover 692. The cover 842 may include a removal body 846 for removing the cover 842 in a similar manner as the removal body 696 shown in FIG. 81.

The pins 844a, b may engage the engagement portion of the expansion apparatus in a similar manner as the respective pins 816a, b shown in FIG. 99. The pins 844a, b may separate the arms from each other to space the compression surfaces of the clip from each other. FIG. 103, for example, illustrates the pins 844a, b engaging respective arms of the engagement portion.

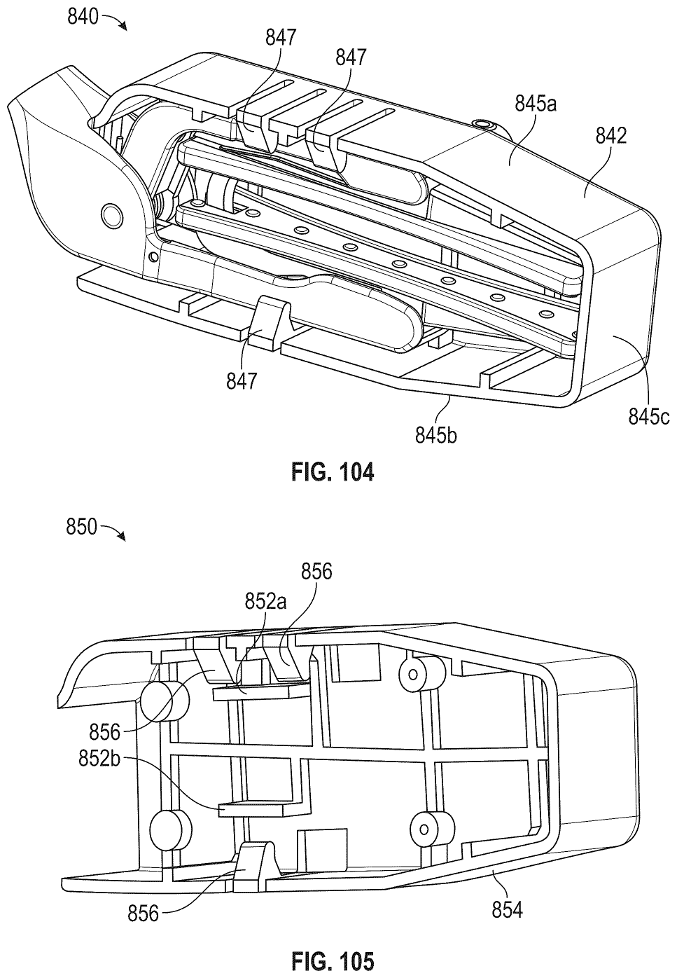

FIG. 104 illustrates a view of the spacer device 840 from an opposite side than shown in FIG. 102. In examples, the spacer device 840 may include protrusions 847 in the form of hooks for engaging the arms of the expansion apparatus on an opposite side than the pins 844a, b. The protrusions 847 may hook over the arms, to impede removal of the spacer device 840 until a desired time, at which the protrusions 847 may be displaced manually for removal of the spacer device 840. Other configurations of spacer devices may be utilized in examples.

FIG. 105, for example, illustrates a variation of a spacer device 850 in which the spacer device 850 includes support bodies 852a, b for supporting respective arms of the engagement portion at a distance from each other. The support bodies 852a, b may comprise substrates for contacting the respective arms that retain the arms at a spacing from each other. The substrates may be positioned beneath the respective arms to maintain the spacing of the arms. The spacer device 850 may include a cover 854 that may be configured similarly as the cover 842 shown in FIG. 102. The spacer device 850 may further include protrusions 856 that may operate in a similar manner as the protrusions 847 shown in FIG. 104.

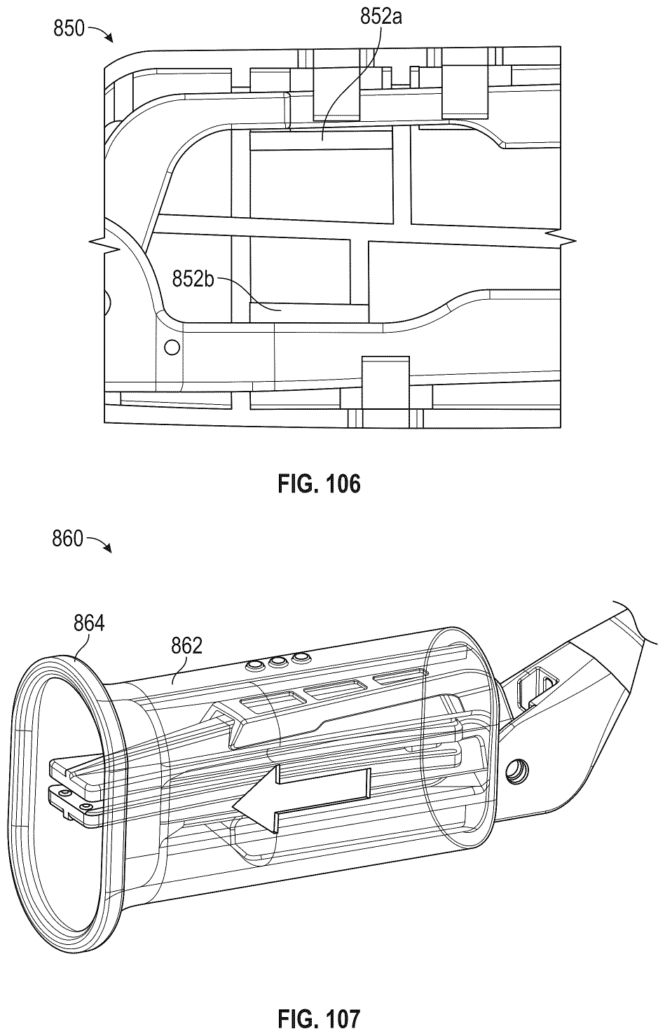

FIG. 106 illustrates a side view of the spacer device 850 coupled to the engagement portion of the expansion apparatus. The arms rest upon the support bodies 852a, b to maintain the spacing of the support bodies 852a, b.

Other configurations of spacer devices may be utilized in examples.

FIGS. 107-118 illustrate examples of spacer devices configured to be axially or longitudinally removed from a clip and a portion of an expansion apparatus. FIG. 107, for example, illustrates a spacer device 860 comprising a cover in the form of a sheath 862 for extending axially or longitudinally along and over a clip and a portion of an expansion apparatus. The sheath 862 may cover the clip and the exterior surfaces of the expansion apparatus. The spacer device 860 may include a removal body 864 in the form of a flared end of the sheath 862.

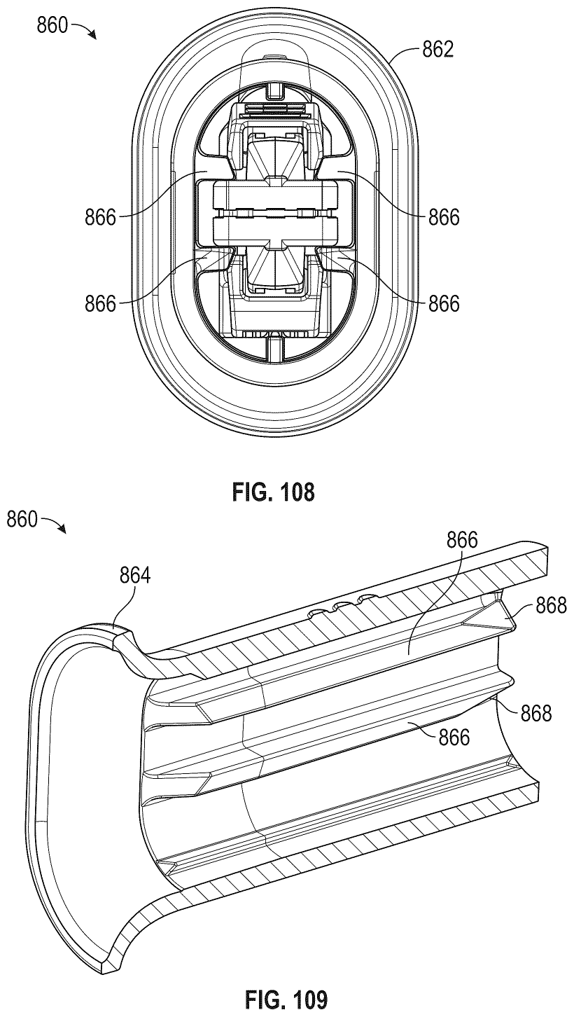

FIG. 108 illustrates an end view of the spacer device 860. The spacer device 860 includes support bodies 866 in the form of rails extending axially or longitudinally along the spacer device 860. The support bodies 866 may be configured to engage the arms of the engagement portion of the expansion apparatus to hold the compression surfaces of the clip at a spacing from each other. The rails may slide relative to the arms of the engagement portion of the expansion apparatus. The arms may contact and rest upon the rails to maintain the spacing between the arms and between the jaws of the clip.

The spacer device 860 may be removed by being slid along a longitudinal dimension of the clip and the engagement portion of the expansion apparatus. The removal body 864 may be gripped by a user and pulled to remove the spacer device 860.

FIG. 109 illustrates a cross sectional view of the spacer device 860 showing the elongate extension of the support bodies 866 comprising rails. The support bodies 866 may include angled or tapered surfaces 868 in examples for improved sliding engagement with the arms of the engagement portion of the expansion apparatus.

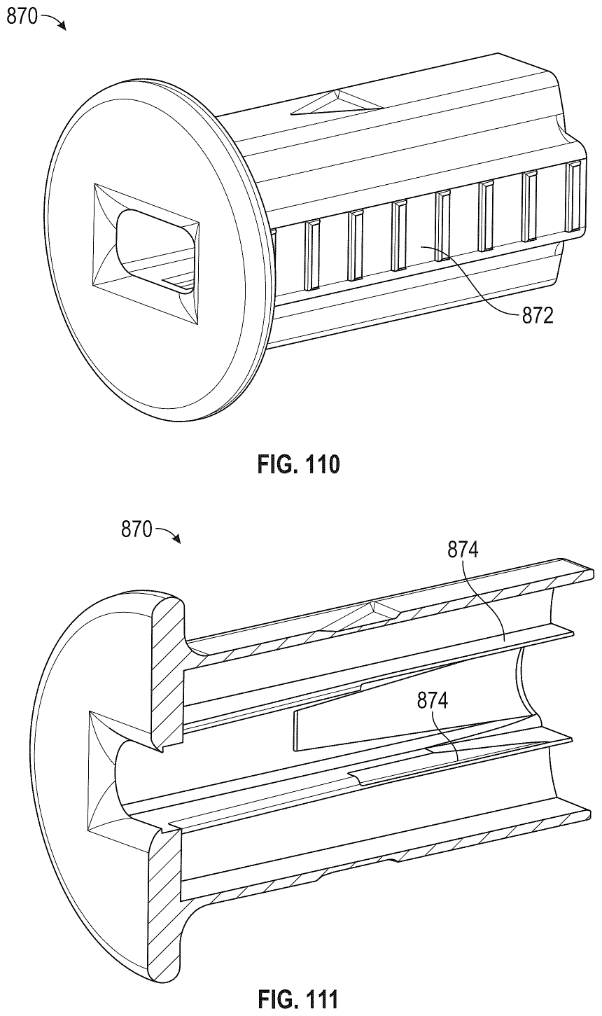

FIG. 110 illustrates a variation of a spacer device 870 including similar features as the spacer device 860, yet having a removal body 872 in the form of a grip surface on a side of the spacer device 870. FIG. 111 illustrates a cross sectional view of the spacer device 870 showing the position of support bodies 874 in the form of rails.

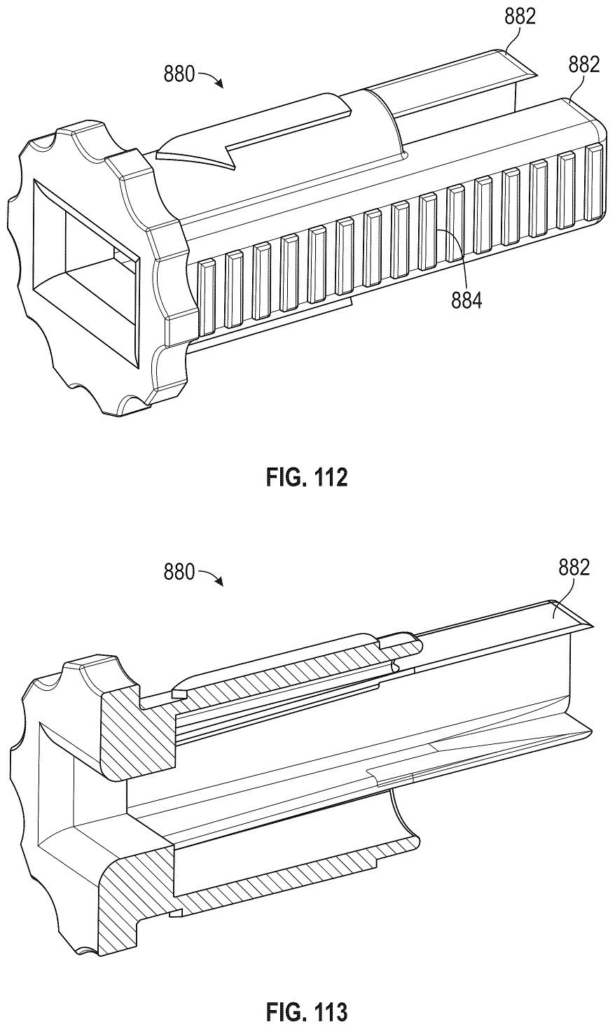

Other variations may be provided. FIG. 112, for example, illustrates a variation of a spacer device 880 including support bodies in the form of support surfaces 882 or rails that are external and uncovered by an outer surface of the spacer device 880. The arms of the expansion apparatus may be positioned upon and rest upon the support surfaces 882 to space the arms from each other and accordingly space the compression surfaces of the clip from each other. The spacer device 880 may include a removal body 884 in the form of a grip surface on a side of the spacer device 880. The removal body 884 may be gripped and pulled longitudinally for removal of the spacer device 880. The arms of the expansion apparatus may slide along the support surfaces 882 comprising rails. FIG. 113 illustrates a cross sectional view of the spacer device 880.

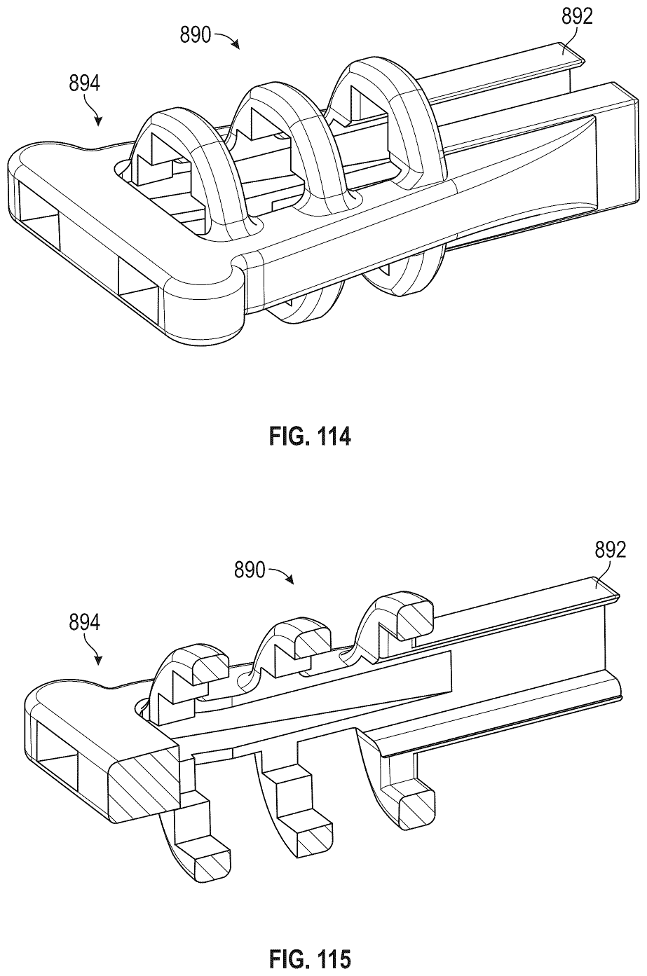

FIG. 114 illustrates a variation of a spacer device 890 including external support surfaces 892 or rails similar to the support surfaces 882 shown in FIG. 113. The spacer device 890, however, may include a removal body 894 in the form of arms protruding axially for grip by a user. The removal body 894 may be gripped and pulled axially for removal of the spacer device 890.

FIG. 115 illustrates a cross sectional view of the spacer device 890.

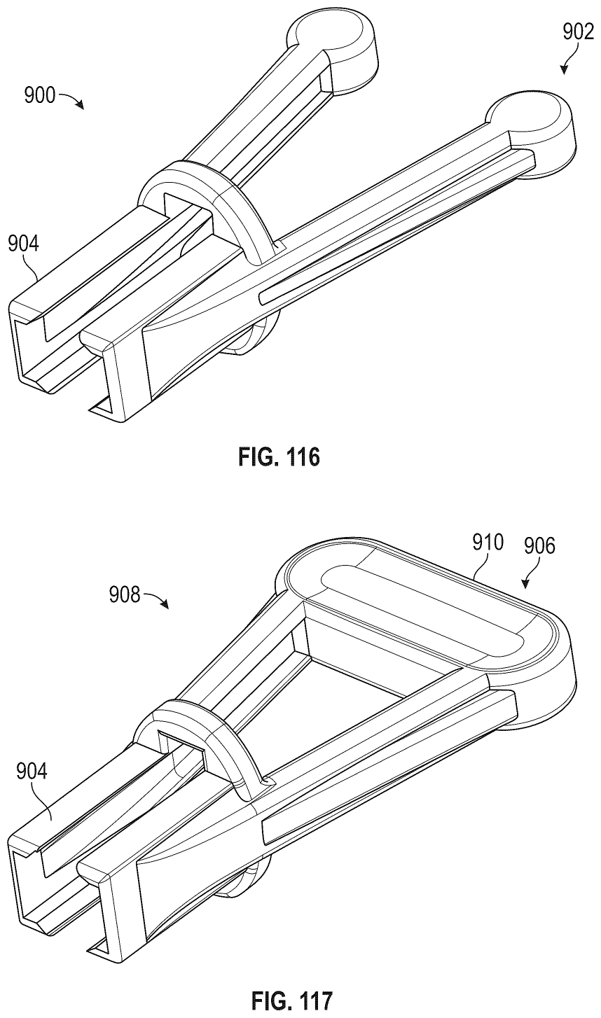

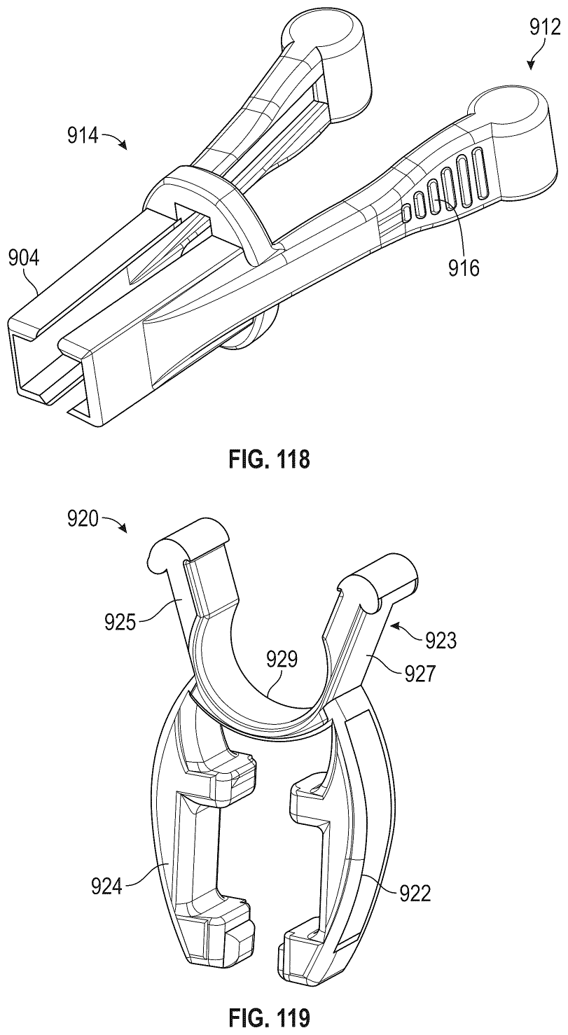

Variations in the configuration of the removal body may be provided in examples. FIG. 116, for example, illustrates a variation comprising a spacer device 900 having a removal body 902 in the form of elongate arms protruding axially or longitudinally. The spacer device 900 may include support surfaces 904 that are similar in operation to the support surfaces 892 shown in FIG. 115. FIG. 117 illustrates a variation in which the arms forming the removal body 906 of the spacer device 908 are coupled to each other with a cross bar 910. FIG. 118 illustrates a variation in which the arms forming the removal body 912 of the spacer device 914 include grip surfaces 916 for gripping by a user.

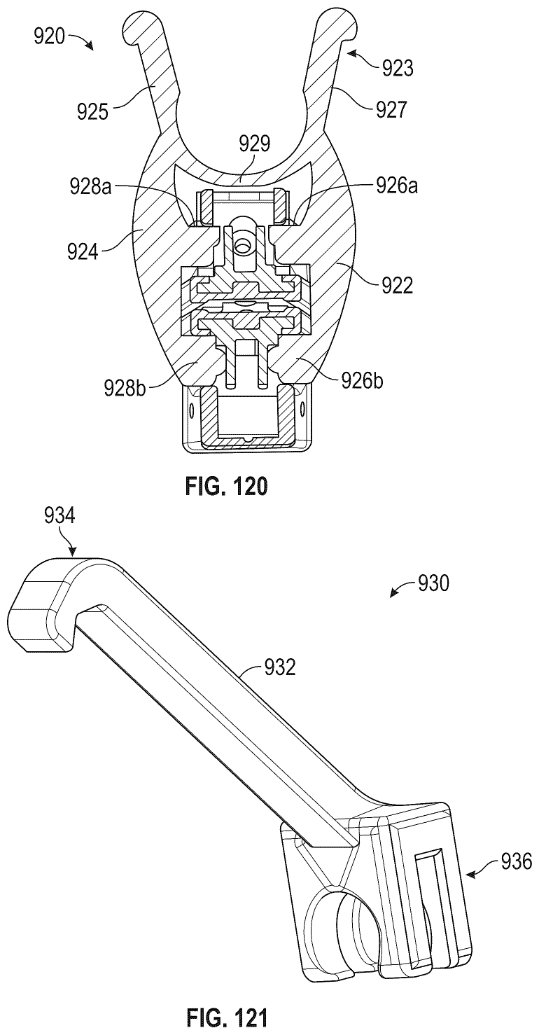

FIGS. 119 and 120 illustrate a variation in which the spacer device 920 includes a plurality of arms 922, 924 for engaging an engagement portion of an expansion apparatus. The arms 922, 924 may be configured to be disposed on opposite sides of arms of an engagement portion of an expansion apparatus for engaging the clip. The first arm 922 may be configured to engage an opposite side of the engagement portion than the second arm 924.

A removal body 923 of the spacer device 920 may be configured to be compressed for release of the spacer device 920. The removal body 923, for example, may comprise arms 925, 927 that are configured to be gripped and pressed together to correspondingly deflect the arms 922, 924 away from each other to release from the expansion apparatus. A biasing body 929 may connect the arms 925, 927 of the removal body 923 to provide a restoring force to drive the arms 922, 924 towards each other.

FIG. 120, for example, illustrates the spacer device 920 upon the expansion apparatus. The arms 922, 924 may include respective support bodies in the form of support surfaces 926a, b, 928a, b for engaging the arms of the engagement portion to space the compression surfaces of the clip from each other. The arms of the engagement portion may rest upon the support surfaces 926a, b, 928a, b.

The removal body 923 may be compressed to release the spacer device 920. The spacer device 920 may be removed in a direction transverse to the axial or longitudinal dimension of the clip at a desired time.

Other configurations may be utilized in examples. FIG. 121, for example, illustrates a configuration of a spacer device 930 including an arm 932. The arm 932 may have a first end portion 934 configured to engage an arm of an expansion apparatus. The arm 932 may have an opposite second end portion 936 configured to engage an anchoring portion of the expansion apparatus. The second end portion 936 may engage an anchoring portion of the expansion apparatus to resist a compressive force between the arms of the engagement portion. The anchoring portion, for example, may comprise an elongate shaft of the expansion apparatus. The first end portion 934 and the second end portion 936 may each comprise couplers.

The coupler at the first end portion 934 may comprise a hook. The coupler at the second end portion 936 may comprise a clip for coupling to the anchoring portion. Other configurations may be utilized in examples.

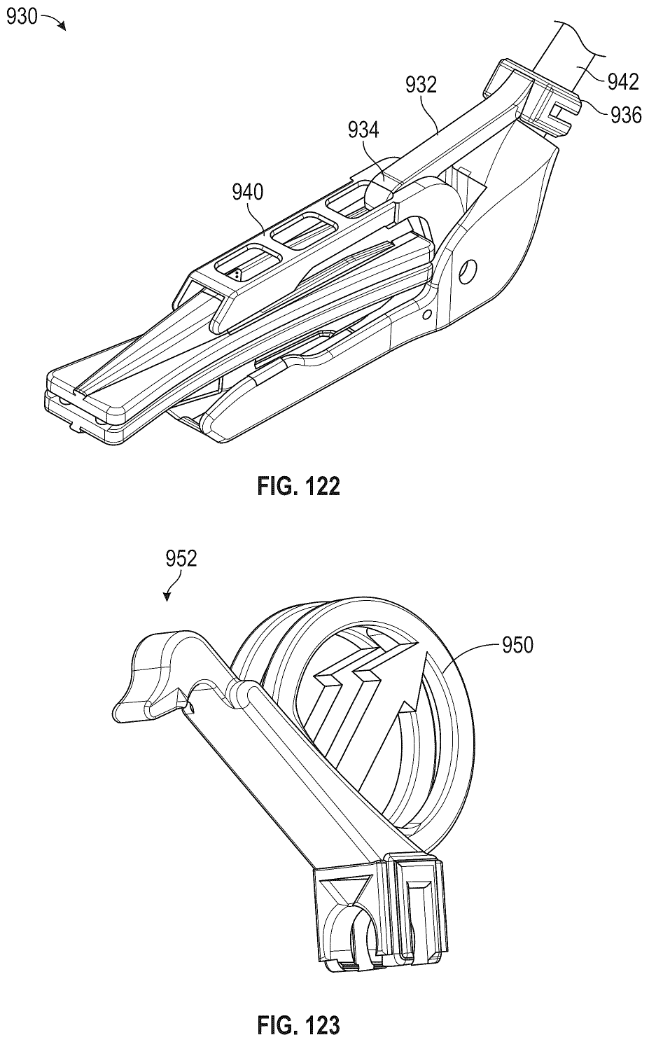

FIG. 122 illustrates the spacer device 930 in position. The first end portion 934 engages an opening or aperture in an arm 940 of the expansion apparatus. The second end portion 936 engages the elongate shaft 942 of the expansion apparatus. The arm 932 of the spacer device 930 may apply a force to the arm 940 of the expansion apparatus to separate the compression surfaces of the clip from each other. In examples, the arm 932 may comprise a removal body for a user to grip and pull for removal of the spacer device 930. Variations in the configuration of the spacer device may be provided.

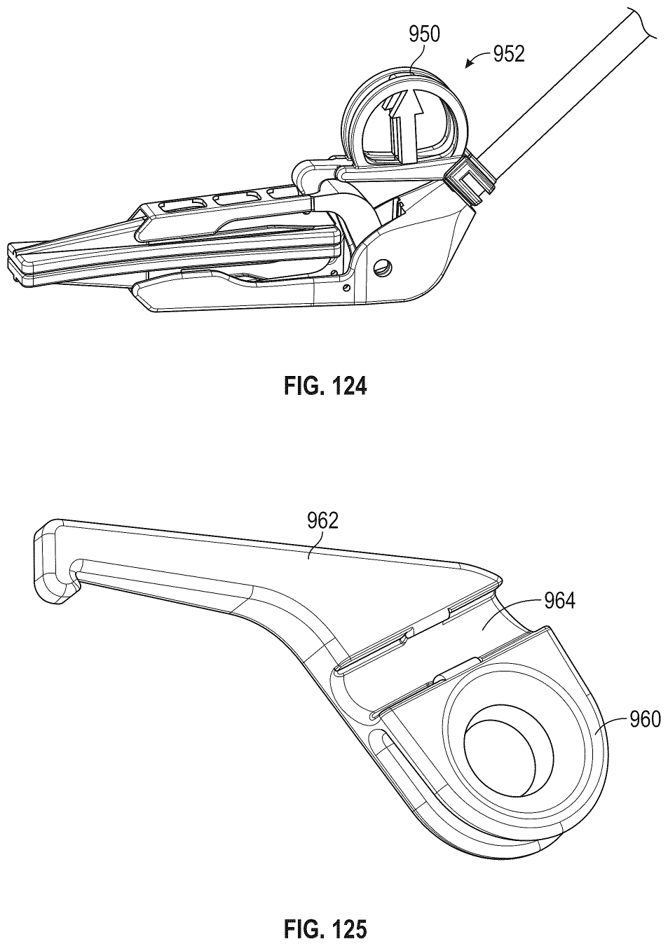

FIG. 123, for example, illustrates a variation in which a ring-shaped removal body 950 is utilized with the spacer device 952. The ring-shaped removal body 950 may be pulled to remove the spacer device 952 from the expansion apparatus. The spacer device 952 may otherwise be configured similarly as the spacer device 930. FIG. 124 illustrates the spacer device 952 in position. Other variations may be provided.

FIG. 125, for example, illustrates a variation in which a removal body 960 comprises a ring-shaped body configured to be positioned on an opposite side of the elongate shaft of the expansion apparatus than the arm 962. The second end portion 964 may include a coupler in the form of a channel for the elongate shaft of the expansion apparatus to pass through. The removal body 960 may be pulled to disengage the second end portion 964 from the anchoring point (e.g., the elongate shaft of the expansion apparatus).

The spacer devices disclosed herein may be retained to the clip and/or an expansion apparatus during a sterilization procedure for the clip and/or expansion apparatus. The spacer device may be assembled with the clip and/or expansion apparatus during a sterilization process, which may comprise e-beam sterilization or other forms of sterilization (e.g., gaseous sterilization).

Other configurations of spacer devices may be utilized in examples. The spacer devices may be utilized with any form of clip and/or expansion apparatus as disclosed herein. The spacer devices may be utilized solely or in combination with any other example disclosed herein.

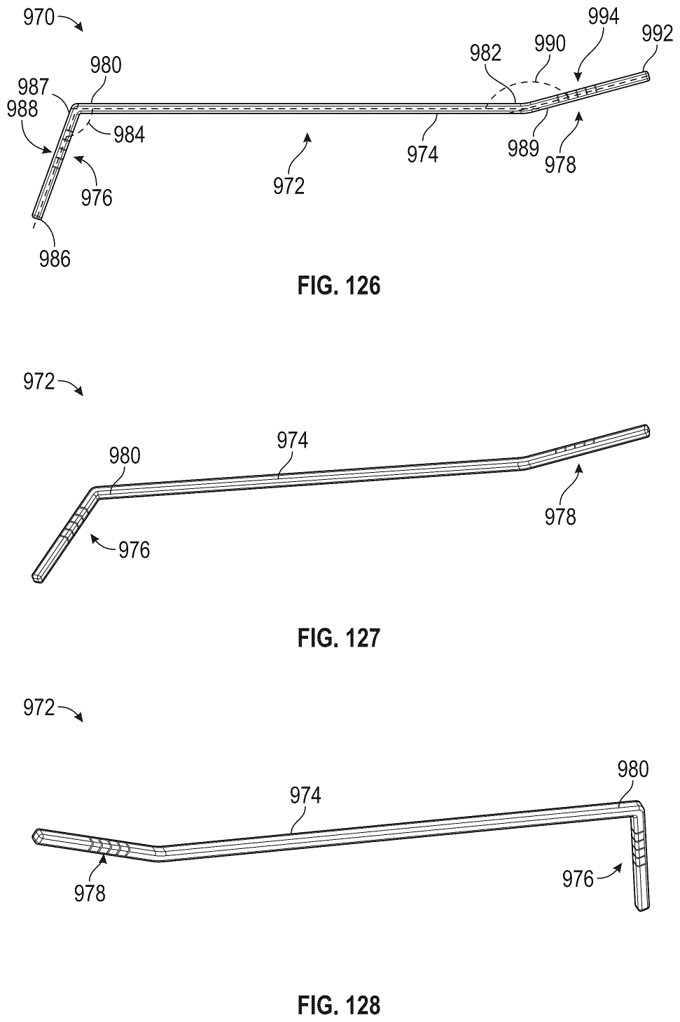

FIGS. 126-128 illustrate an example of a sizer 970 that may be utilized for sizing a portion of a heart. The sizer 970 may include an elongate shaft 972 having a central shaft portion 974, a first end portion 976, and a second end portion 978 that is opposite the first end portion 976.

The central shaft portion 974 may include a first end 980 and a second end 982 that is opposite the first end 980. The central shaft portion 974 may comprise a linear shaft that is straight and has a uniform diameter.

The first end portion 976 may be coupled to the first end 980 of the central shaft portion 974 and may extend from the first end 980 at an angle 984. The first end portion 976 may comprise a linear shaft that is straight from the first end 980 of the central shaft portion 974 to the opposite end 986 or tip of the first end portion 976. The first end portion 976 may include an end 987 that couples to the first end 980 of the central shaft portion 974 and forms the angle 984 with the first end 980 of the central shaft portion 974. The first end portion 976 may have a uniform diameter and may have the diameter of the central shaft portion 974.

In examples, the first end portion 976 may include one or more indicators 988 that may be configured to indicate a size of the portion of the heart being sized by the sizer 970. The indicators 988 may be positioned on multiple side surfaces of the first end portion 976 (with three side surfaces including the indicators 988 as represented in FIGS. 126-128).

The indicators 988 may be configured such that the size of the portion of the heart is indicated as a distance from the end 986 or tip of the first end portion 976.

The second end portion 978 may be coupled to the second end 982 of the central shaft portion 974 and may extend from the second end 982 at an angle 990. The angle 990 may be different than the angle 984. As such, varied entry angles for use of the sizer 970 may be provided (with a different angle provided for use of the second end portion 978 relative to the first end portion 976). The angle 990 may be non-perpendicular, and the angle 984 may be non-perpendicular. In examples, the angle 990 of the second end portion 978 may be between 140 and 180 degrees. In examples, the angle 990 of the second end portion 978 may be about 165 degrees. In examples, the angle 984 of the first end portion 976 may be between 90 and 130 degrees. In examples, the angle 984 of the first end portion 976 may be about 110 degrees. Other angles may be utilized as desired.

The second end portion 978 may comprise a linear shaft that is straight from the second end 982 of the central shaft portion 974 to the opposite end 992 or tip of the second end portion 978. The second end portion 978 may include an end 989 that couples to the second end 982 of the central shaft portion 974 and forms the angle 990 with the second end 982 of the central shaft portion 974. The second end portion 978 may have a uniform diameter and may have the diameter of the central shaft portion 974.

In examples, the second end portion 978 may include one or more indicators 994 that may be configured to indicate a size of the portion of the heart being sized by the sizer 970. The indicators 994 may be positioned on multiple side surfaces of the second end portion 978 (with three side surfaces including the indicators 994 as represented in FIGS. 126-128). The indicators 994 may be configured such that the size of the portion of the heart is indicated as a distance from the end 992 or tip of the second end portion 978.

The indicators 988, 994 may each be laser cut, engraved, or otherwise applied to the respective portion of the sizer 970. The indicators 988, 994 may be configured to be light reflective, to enhance ease of visibility during a deployment procedure. The indicators 988, 994 may comprise one or more of hash lines or graduation lines, or numerals indicating a distance, or combination thereof. Other configurations of indicators 988, 994 may be utilized in examples.

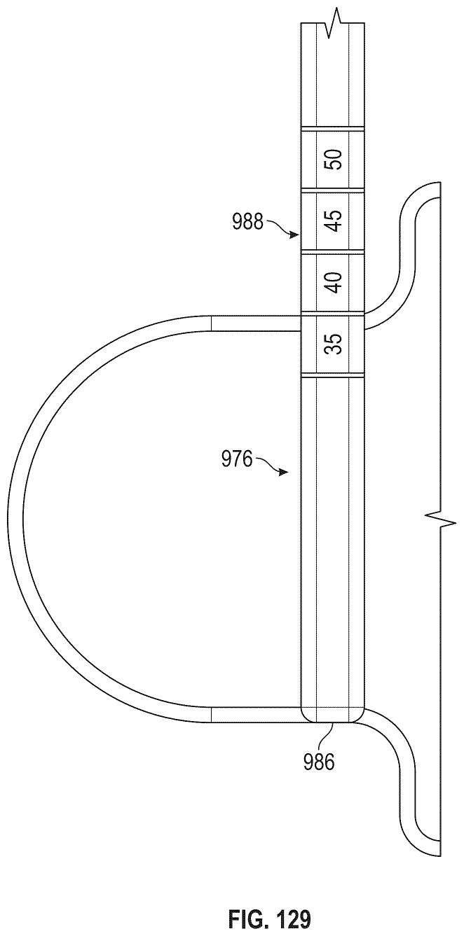

In examples, the indicators 988 of the first end portion 976 or the indicators 994 of the second end portion 978 may be selected to size the portion of the heart. The respective first end portion 976 or second end portion 978 may be oriented along the portion of the heart to be sized. FIG. 129, for example, illustrates the first end portion 976 of the sizer 970 positioned along and parallel with the ostium of the left atrial appendage (LAA). As such, the diameter of the ostium may be determined and an appropriate size of clip may be selected for deployment to occlude the LAA. Alternatively, the second end portion 978 may be utilized for measurement. The position of the indicators 988, 994 on multiple side surfaces of the sizer 970 may provide for varied angles of entry and use of the sizer 970.

The sizer may be utilized solely or in combination with any example disclosed herein.

Various other modifications of the clips disclosed herein may be provided. Various other methods of deployment and use of the clips may be provided as desired.

The clips as disclosed herein may be utilized to close the LAA or may be utilized to close another portion of a heart. In examples, the clips may be utilized to close other portions of a body, including other tubular vessels or other portions of a body. Deployment may be via a delivery apparatus or via another method as desired. Deployment may be via surgical methods and may be transcatheter or via non-invasive surgery in methods.

Variations in the clips and methods disclosed herein may be provided. Features across examples may be combined. Features may be excluded or added to in various examples disclosed herein. Combinations of features of examples may be provided. The clips may be utilized solely or in methods disclosed herein, or in combination with other devices disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: An expansion apparatus for a left atrial appendage clip, the expansion apparatus comprising: an elongate shaft having a first end portion and a second end portion; an engagement portion positioned at the first end portion of the elongate shaft and having at least two arms, the at least two arms including a first arm extending longitudinally and for sliding engagement with a first jaw of the left atrial appendage clip and a second arm extending longitudinally and for sliding engagement with a second jaw of the left atrial appendage clip; and a retention mechanism configured to retain the left atrial appendage clip to the engagement portion to impede sliding release of the left atrial appendage clip from the first arm and the second arm.

Example 2: The expansion apparatus of any example herein, in particular Example 1, wherein the first arm includes at least one rail for sliding engagement with the first jaw, and the second arm includes at least one rail for sliding engagement with the second jaw.

Example 3: The expansion apparatus of any example herein, in particular Example 2, wherein the first arm includes a channel for receiving at least a portion of the first jaw, and the second arm includes a channel for receiving at least a portion of the second jaw.

Example 4: The expansion apparatus of any example herein, in particular Examples 1-3, wherein the first arm is configured to move relative to the second arm to open or close the left atrial appendage clip.

Example 5: The expansion apparatus of any example herein, in particular Example 4, wherein the first arm is configured to move away from the second arm to open the left atrial appendage clip and is configured to move towards the second arm to close the left atrial appendage clip.

Example 6: The expansion apparatus of any example herein, in particular Examples 1-5, wherein the first arm and the second arm each include a distal end portion and a proximal end portion, each of the distal end portions including an opening for sliding release of the left atrial appendage clip from the respective first arm and second arm, and each of the proximal end portions being coupled to a base.

Example 7: The expansion apparatus of any example herein, in particular Example 6, wherein at least one of the first arm or the second arm is pivotally coupled to the base.

Example 8: The expansion apparatus of any example herein, in particular Examples 1-7, wherein the retention mechanism is configured to provide a force from the first arm to the first jaw to retain the left atrial appendage clip to the engagement portion to impede sliding release of the left atrial appendage clip from the first arm and the second arm.

Example 9: The expansion apparatus of any example herein, in particular Examples 1-8, wherein the retention mechanism is configured to provide an expansion force to the first jaw to retain the left atrial appendage clip to the engagement portion to impede sliding release of the left atrial appendage clip from the first arm and the second arm.

Example 10: The expansion apparatus of any example herein, in particular Examples 1-9, wherein the retention mechanism is configured to provide a friction force to the first jaw to retain the left atrial appendage clip to the engagement portion to impede sliding release of the left atrial appendage clip from the first arm and the second arm.

Example 11: The expansion apparatus of any example herein, in particular Examples 1-10, wherein the retention mechanism is configured to position the first arm and the second arm in an at least partially opened configuration to retain the left atrial appendage clip to the engagement portion to impede sliding release of the left atrial appendage clip from the first arm and the second arm.

Example 12: The expansion apparatus of any example herein, in particular Examples 1-11, wherein the retention mechanism is configured to provide a preload force upon the left atrial appendage clip to retain the left atrial appendage clip to the engagement portion to impede sliding release of the left atrial appendage clip from the first arm and the second arm.

Example 13: The expansion apparatus of any example herein, in particular Examples 1-12, wherein the first arm includes a first rail for sliding engagement with the first jaw, and the retention mechanism is configured to provide an interference fit between the first rail and at least a portion of the left atrial appendage clip to retain the left atrial appendage clip to the engagement portion to impede sliding release of the left atrial appendage clip from the first arm and the second arm.

Example 14: The expansion apparatus of any example herein, in particular Example 13, wherein the retention mechanism is configured to angle the first rail relative to a rail surface of the first jaw to provide the interference fit.

Example 15: The expansion apparatus of any example herein, in particular Examples 1-14, wherein the retention mechanism includes a barrier on the first arm configured to impede sliding release of the left atrial appendage clip from the first arm.

Example 16: The expansion apparatus of any example herein, in particular Example 15, wherein the barrier is configured to displace relative to at least a portion of the left atrial appendage clip to allow for sliding release of the left atrial appendage clip from the first arm.

Example 17: The expansion apparatus of any example herein, in particular Examples 1-16, wherein the retention mechanism is configured to release the left atrial appendage clip from the first arm and the second arm in response to a force of occlusion being applied to the left atrial appendage clip.

Example 18: The expansion apparatus of any example herein, in particular Examples 1-17, wherein the first arm and the second arm are configured to be positioned at a first angle relative to each other for release of the left atrial appendage clip from the first arm and the second arm, and the retention mechanism is configured to retain the first arm and the second arm at a second angle relative to each other that is greater than the first angle.

Example 19: The expansion apparatus of any example herein, in particular Examples 1-18, wherein the retention mechanism is actuatable.

Example 20: The expansion apparatus of any example herein, in particular Examples 1-19, wherein the retention mechanism is user actuatable.

Example 21: The expansion apparatus of any example herein, in particular Examples 1-20, wherein the retention mechanism is selectively actuatable from a retention configuration in which the retention mechanism impedes sliding release of the left atrial appendage clip from the first arm and the second arm, to a release configuration in which the retention mechanism allows for sliding release of the left atrial appendage clip from the first arm and the second arm.

Example 22: The expansion apparatus of any example herein, in particular Example 21, further comprising a control device operable by a user to selectively actuate the retention mechanism.

Example 23: The expansion apparatus of any example herein, in particular Example 22, wherein the control device comprises one or more of a lever, a slider, or a knob.

Example 24: The expansion apparatus of any example herein, in particular Examples 21-23, further comprising a lock mechanism for locking the retention mechanism in the retention configuration and locking the retention mechanism in the release configuration.

Example 25: The expansion apparatus of any example herein, in particular Examples 1-24, further comprising a control mechanism for controlling opening and closing of the first arm relative to the second arm, and the retention mechanism is configured to apply a force to a least a portion of the control mechanism to impede sliding release of the left atrial appendage clip from the first arm and the second arm.

Example 26: The expansion apparatus of any example herein, in particular Example 25, wherein the retention mechanism includes a cam body configured to apply a force to at least a portion of the control mechanism.

Example 27: The expansion apparatus of any example herein, in particular Example 26, wherein the control mechanism includes a control lever arm and the cam body is configured to apply a force to at least a portion of the control lever arm.

Example 28: The expansion apparatus of any example herein, in particular Examples 25-27, wherein the control mechanism includes a control lever arm and the retention mechanism includes a slide body configured to apply a force to at least a portion of the control lever arm.

Example 29: The expansion apparatus of any example herein, in particular Examples 1-28, wherein the retention mechanism includes a stopper configured to impede pivotal movement of the first arm or the second arm.

Example 30: The expansion apparatus of any example herein, in particular Examples 1-29, wherein the retention mechanism includes at least one protrusion configured to engage at least a portion of the left atrial appendage clip to impede sliding release of the left atrial appendage clip from the first arm and the second arm.

Example 31: The expansion apparatus of any example herein, in particular Example 30, wherein the at least one protrusion includes a hook.

Example 32: The expansion apparatus of any example herein, in particular

Example 30 or Example 31, wherein the at least one protrusion is spring biased against at least a portion of the left atrial appendage clip.

Example 33: The expansion apparatus of any example herein, in particular Examples 30-32, wherein the at least one protrusion is selectively actuatable from a retention configuration in which the at least one protrusion impedes sliding release of the left atrial appendage clip from the first arm and the second arm, to a release configuration in which the at least one protrusion allows for sliding release of the left atrial appendage clip from the first arm and the second arm.

Example 34: The expansion apparatus of any example herein, in particular Example 33, wherein the retention mechanism includes a lever arm and a tether, the at least one protrusion being coupled to the lever arm, and the tether configured to apply a force to the lever arm to actuate the at least one protrusion from the retention configuration to the release configuration.

Example 35: The expansion apparatus of any example herein, in particular Example 34, wherein the retention mechanism includes a force dampener for dampening a force applied to the tether.

Example 36: The expansion apparatus of any example herein, in particular Examples 1-35, further comprising a handle positioned at the second end portion of the elongate shaft.

Example 37: The expansion apparatus of any example herein, in particular Example 36, further comprising a control mechanism for controlling opening and closing of the first arm relative to the second arm and including a control device positioned upon the handle.

Example 38: The expansion apparatus of any example herein, in particular Example 37, wherein the control device is a lever.

Example 39: The expansion apparatus of any example herein, in particular Examples 1-38, further comprising a rotation mechanism for controlling rotation of the engagement portion about a longitudinal axis of the elongate shaft.

Example 40: The expansion apparatus of any example herein, in particular Example 39, wherein the rotation mechanism includes a lock for selectively locking a rotational position of the engagement portion about the longitudinal axis of the elongate shaft.

Example 41: A method comprising: utilizing an expansion apparatus to deploy or capture a left atrial appendage clip, the expansion apparatus including: an elongate shaft having a first end portion and a second end portion, an engagement portion positioned at the first end portion of the elongate shaft and having at least two arms, the at least two arms including a first arm extending longitudinally and for sliding engagement with a first jaw of the left atrial appendage clip and a second arm extending longitudinally and for sliding engagement with a second jaw of the left atrial appendage clip, and a retention mechanism configured to retain the left atrial appendage clip to the engagement portion to impede sliding release of the left atrial appendage clip from the first arm and the second arm.

Example 42: The method of any example herein, in particular Example 41, wherein the first arm includes at least one rail for sliding engagement with the first jaw, and the second arm includes at least one rail for sliding engagement with the second jaw.

Example 43: The method of any example herein, in particular Example 42, wherein the first arm includes a channel for receiving at least a portion of the first jaw, and the second arm includes a channel for receiving at least a portion of the second jaw.

Example 44: The method of any example herein, in particular Examples 41-43, wherein the first arm is configured to move relative to the second arm to open or close the left atrial appendage clip.

Example 45: The method of any example herein, in particular Example 44, wherein the first arm is configured to move away from the second arm to open the left atrial appendage clip and is configured to move towards the second arm to close the left atrial appendage clip.

Example 46: The method of any example herein, in particular Examples 41-45, wherein the first arm and the second arm each include a distal end portion and a proximal end portion, each of the distal end portions including an opening for sliding release of the left atrial appendage clip from the respective first arm and second arm, and each of the proximal end portions being coupled to a base.

Example 47: The method of any example herein, in particular Example 46, wherein at least one of the first arm or the second arm is pivotally coupled to the base.

Example 48: The method of any example herein, in particular Examples 41-47, wherein the retention mechanism is configured to provide a force from the first arm to the first jaw to retain the left atrial appendage clip to the engagement portion to impede sliding release of the left atrial appendage clip from the first arm and the second arm.

Example 49: The method of any example herein, in particular Examples 41-48, wherein the retention mechanism is configured to provide an expansion force to the first jaw to retain the left atrial appendage clip to the engagement portion to impede sliding release of the left atrial appendage clip from the first arm and the second arm.

Example 50: The method of any example herein, in particular Examples 41-49, wherein the retention mechanism is configured to provide a friction force to the first jaw to retain the left atrial appendage clip to the engagement portion to impede sliding release of the left atrial appendage clip from the first arm and the second arm.

Example 51: The method of any example herein, in particular Examples 41-50, wherein the retention mechanism is configured to position the first arm and the second arm in an at least partially opened configuration to retain the left atrial appendage clip to the engagement portion to impede sliding release of the left atrial appendage clip from the first arm and the second arm.

Example 52: The method of any example herein, in particular Examples 41-51, wherein the retention mechanism is configured to provide a preload force upon the left atrial appendage clip to retain the left atrial appendage clip to the engagement portion to impede sliding release of the left atrial appendage clip from the first arm and the second arm.

Example 53: The method of any example herein, in particular Examples 41-52, wherein the first arm includes a first rail for sliding engagement with the first jaw, and the retention mechanism is configured to provide an interference fit between the first rail and at least a portion of the left atrial appendage clip to retain the left atrial appendage clip to the engagement portion to impede sliding release of the left atrial appendage clip from the first arm and the second arm.

Example 54: The method of any example herein, in particular Example 53, wherein the retention mechanism is configured to angle the first rail relative to a rail surface of the first jaw to provide the interference fit.

Example 55: The method of any example herein, in particular Examples 41-54, wherein the retention mechanism includes a barrier on the first arm configured to impede sliding release of the left atrial appendage clip from the first arm.

Example 56: The method of any example herein, in particular Example 55, wherein the barrier is configured to displace relative to at least a portion of the left atrial appendage clip to allow for sliding release of the left atrial appendage clip from the first arm.

Example 57: The method of any example herein, in particular Examples 41-56, wherein the retention mechanism is configured to release the left atrial appendage clip from the first arm and the second arm in response to a force of occlusion being applied to the left atrial appendage clip.

Example 58: The method of any example herein, in particular Examples 41-57, wherein the first arm and the second arm are configured to be positioned at a first angle relative to each other for release of the left atrial appendage clip from the first arm and the second arm, and the retention mechanism is configured to retain the first arm and the second arm at a second angle relative to each other that is greater than the first angle.

Example 59: The method of any example herein, in particular Examples 41-58, wherein the retention mechanism is actuatable.

Example 60: The method of any example herein, in particular Examples 41-59, wherein the retention mechanism is user actuatable.

Example 61: The method of any example herein, in particular Examples 41-60, wherein the retention mechanism is selectively actuatable from a retention configuration in which the retention mechanism impedes sliding release of the left atrial appendage clip from the first arm and the second arm, to a release configuration in which the retention mechanism allows for sliding release of the left atrial appendage clip from the first arm and the second arm.

Example 62: The method of any example herein, in particular Example 61, wherein a control device is operable by a user to selectively actuate the retention mechanism.

Example 63: The method of any example herein, in particular Example 62, wherein the control device comprises one or more of a lever, a slider, or a knob.

Example 64: The method of any example herein, in particular Examples 61-63, further comprising a lock mechanism for locking the retention mechanism in the retention configuration and locking the retention mechanism in the release configuration.

Example 65: The method of any example herein, in particular Examples 41-64, wherein a control mechanism is for controlling opening and closing of the first arm relative to the second arm, and the retention mechanism is configured to apply a force to a least a portion of the control mechanism to impede sliding release of the left atrial appendage clip from the first arm and the second arm.

Example 66: The method of any example herein, in particular Example 65, wherein the retention mechanism includes a cam body configured to apply a force to at least a portion of the control mechanism.

Example 67: The method of any example herein, in particular Example 66, wherein the control mechanism includes a control lever arm and the cam body is configured to apply a force to at least a portion of the control lever arm.

Example 68: The method of any example herein, in particular Examples 65-67, wherein the control mechanism includes a control lever arm and the retention mechanism includes a slide body configured to apply a force to at least a portion of the control lever arm.

Example 69: The method of any example herein, in particular Examples 41-68, wherein the retention mechanism includes a stopper configured to impede pivotal movement of the first arm or the second arm.

Example 70: The method of any example herein, in particular Examples 41-69, wherein the retention mechanism includes at least one protrusion configured to engage at least a portion of the left atrial appendage clip to impede sliding release of the left atrial appendage clip from the first arm and the second arm.

Example 71: The method of any example herein, in particular Example 70, wherein the at least one protrusion includes a hook.

Example 72: The method of any example herein, in particular Example 70 or Example 71, wherein the at least one protrusion is spring biased against at least a portion of the left atrial appendage clip.

Example 73: The method of any example herein, in particular Examples 70-72, wherein the at least one protrusion is selectively actuatable from a retention configuration in which the at least one protrusion impedes sliding release of the left atrial appendage clip from the first arm and the second arm, to a release configuration in which the at least one protrusion allows for sliding release of the left atrial appendage clip from the first arm and the second arm.

Example 74: The method of any example herein, in particular Example 73, wherein the retention mechanism includes a lever arm and a tether, the at least one protrusion being coupled to the lever arm, and the tether configured to apply a force to the lever arm to actuate the at least one protrusion from the retention configuration to the release configuration.

Example 75: The method of any example herein, in particular Example 74, wherein the retention mechanism includes a force dampener for dampening a force applied to the tether.

Example 76: The method of any example herein, in particular Examples 41-75, wherein a handle is positioned at the second end portion of the elongate shaft.

Example 77: The method of any example herein, in particular Example 76, wherein a control mechanism is for controlling opening and closing of the first arm relative to the second arm and including a control device positioned upon the handle.

Example 78: The method of any example herein, in particular Example 77, wherein the control device is a lever.

Example 79: The method of any example herein, in particular Examples 41-78, wherein a rotation mechanism is for controlling rotation of the engagement portion about a longitudinal axis of the elongate shaft.

Example 80: The method of any example herein, in particular Example 79, wherein the rotation mechanism includes a lock for selectively locking a rotational position of the engagement portion about the longitudinal axis of the elongate shaft.

Example 81: An expansion apparatus for a left atrial appendage clip, the expansion apparatus comprising: an elongate shaft having a first end portion and a second end portion; and an engagement portion positioned at the first end portion of the elongate shaft and having at least two arms each extending from a base, the at least two arms including: a first arm extending longitudinally and for sliding engagement with a first jaw of the left atrial appendage clip and having a proximal end portion fixedly coupled to the base, and a second arm extending longitudinally and for sliding engagement with a second jaw of the left atrial appendage clip and having a proximal end portion pivotally coupled to the base, the second arm configured to pivot at the base to open or close the left atrial appendage clip.

Example 82: The expansion apparatus of any example herein, in particular Example 81, wherein the first arm includes at least one rail for sliding engagement with the first jaw, and the second arm includes at least one rail for sliding engagement with the second jaw.

Example 83: The expansion apparatus of any example herein, in particular Example 81 or Example 82, wherein the first arm includes a channel for receiving at least a portion of the first jaw, and the second arm includes a channel for receiving at least a portion of the second jaw.

Example 84: The expansion apparatus of any example herein, in particular Examples 81-83, wherein the first arm is configured to move away from the second arm to open the left atrial appendage clip and is configured to move towards the second arm to close the left atrial appendage clip.

Example 85: The expansion apparatus of any example herein, in particular Examples 81-84, further comprising a retention mechanism configured to retain the left atrial appendage clip to the engagement portion to impede sliding release of the left atrial appendage clip from the first arm and the second arm.

Example 86: A method comprising: utilizing an expansion apparatus to deploy or capture a left atrial appendage clip, the expansion apparatus including: an elongate shaft having a first end portion and a second end portion, and an engagement portion positioned at the first end portion of the elongate shaft and having at least two arms each extending from a base, the at least two arms including: a first arm extending longitudinally and for sliding engagement with a first jaw of the left atrial appendage clip and having a proximal end portion fixedly coupled to the base, and a second arm extending longitudinally and for sliding engagement with a second jaw of the left atrial appendage clip and having a proximal end portion pivotally coupled to the base, the second arm configured to pivot at the base to open or close the left atrial appendage clip.

Example 87: The method of any example herein, in particular Example 86, wherein the first arm includes at least one rail for sliding engagement with the first jaw, and the second arm includes at least one rail for sliding engagement with the second jaw.

Example 88: The method of any example herein, in particular Example 86 or Example 87, wherein the first arm includes a channel for receiving at least a portion of the first jaw, and the second arm includes a channel for receiving at least a portion of the second jaw.

Example 89: The method of any example herein, in particular Examples 86-88, wherein the first arm is configured to move away from the second arm to open the left atrial appendage clip and is configured to move towards the second arm to close the left atrial appendage clip.

Example 90: The method of any example herein, in particular Examples 86-89, wherein a retention mechanism is configured to retain the left atrial appendage clip to the engagement portion to impede sliding release of the left atrial appendage clip from the first arm and the second arm.

Example 91: A clip for a portion of a heart, the clip comprising: a first jaw extending from a first end portion to a second end portion along a length of the first jaw, the first jaw including a first compression surface having a concave curvature relative to the first jaw; a second jaw extending from a first end portion to a second end portion along a length of the second jaw, the second jaw including a second compression surface having a concave curvature relative to the second jaw; and a spring configured to force the first jaw and the second jaw together to compress the portion of the heart between the first compression surface and the second compression surface.

Example 92: The clip of any example herein, in particular Example 91, wherein the first compression surface is spaced from the second compression surface with a gap when the clip is in a closed configuration.

Example 93: The clip of any example herein, in particular Example 91 or Example 92, wherein the first compression surface includes a first end portion and a second end portion, and the second compression surface includes a first end portion and a second end portion, and the first end portion of the first compression surface contacts the first end portion of the second compression surface when the clip is in a closed configuration, and the second end portion of the first compression surface contacts the second end portion of the second compression surface when the clip is in a closed configuration.

Example 94: The clip of any example herein, in particular Examples 91-93, wherein the clip includes one or more elongate couplers positioned on one or more of the first jaw or the second jaw and extending along the length of the respective first jaw or second jaw, the one or more elongate couplers configured to engage an expansion apparatus for the clip.

Example 95: The clip of any example herein, in particular Example 94, wherein the one or more elongate couplers comprise one or more channels.

Example 96: The clip of any example herein, in particular Example 94 or Example 95, wherein the one or more elongate couplers comprise one or more protrusions.

Example 97: The clip of any example herein, in particular Example 96, wherein the one or more protrusions form one or more rails extending along the length of the respective first jaw or second jaw.

Example 98: The clip of any example herein, in particular Examples 94-97, wherein the second end portion of the first jaw and the second end portion of the second jaw form an open end of the clip, and the first end portion of the first jaw and the first end portion of the second jaw form a closed end of the clip, and the one or more elongate couplers are configured to engage the expansion apparatus advanced in a direction from the closed end of the clip towards the open end of the clip.

Example 99: The clip of any example herein, in particular Examples 94-98, wherein the one or more elongate couplers are configured to slidably engage with the expansion apparatus.

Example 100: The clip of any example herein, in particular Examples 91-99, wherein the spring includes a loop extending towards the first end portion of the first jaw and the first end portion of the second jaw.

Example 101: The clip of any example herein, in particular Examples 91-100, wherein the first jaw and the second jaw form a compression channel between the first jaw and the second jaw, the compression channel having an opening at the second end portion of the first jaw and the second end portion of the second jaw, and the compression channel being closed at the first end portion of the first jaw and the first end portion of the second jaw.

Example 102: The clip of any example herein, in particular Example 101, wherein the spring closes the compression channel at the first end portion of the first jaw and the first end portion of the second jaw.

Example 103: The clip of any example herein, in particular Examples 91-102, wherein the first jaw includes: a first end surface positioned at the first end portion of the first jaw, a second end surface positioned at the second end portion of the first jaw, an outer surface facing opposite the first compression surface, a first side surface extending from the first compression surface to the outer surface of the first jaw, and a second side surface extending from the first compression surface to the outer surface of the first jaw and facing opposite the first side surface.

Example 104: The clip of any example herein, in particular Example 103, wherein the first side surface is curved concave relative to the first jaw.

Example 105: The clip of any example herein, in particular Examples 91-104, wherein the clip is configured to occlude a left atrial appendage.

Example 106: A method comprising: deploying a clip to close a portion of a heart, the clip including: a first jaw extending from a first end portion to a second end portion along a length of the first jaw, the first jaw including a first compression surface having a concave curvature relative to the first jaw, a second jaw extending from a first end portion to a second end portion along a length of the second jaw, the second jaw including a second compression surface having a concave curvature relative to the second jaw, and a spring configured to force the first jaw and the second jaw together to compress the portion of the heart between the first compression surface and the second compression surface.

Example 107: The method of any example herein, in particular Example 106, wherein the first compression surface is spaced from the second compression surface with a gap when the clip is in a closed configuration.

Example 108: The method of any example herein, in particular Example 106 or Example 107, wherein the first compression surface includes a first end portion and a second end portion, and the second compression surface includes a first end portion and a second end portion, and the first end portion of the first compression surface contacts the first end portion of the second compression surface when the clip is in a closed configuration, and the second end portion of the first compression surface contacts the second end portion of the second compression surface when the clip is in a closed configuration.

Example 109: The method of any example herein, in particular Examples 106-108, wherein the clip includes one or more elongate couplers positioned on one or more of the first jaw or the second jaw and extending along the length of the respective first jaw or second jaw, the one or more elongate couplers configured to engage an expansion apparatus for the clip.

Example 110: The method of any example herein, in particular Example 109, wherein the one or more elongate couplers comprise one or more channels.

Example 111: The method of any example herein, in particular Example 109 or Example 110, wherein the one or more elongate couplers comprise one or more protrusions.

Example 112: The method of any example herein, in particular Example 111, wherein the one or more protrusions form one or more rails extending along the length of the respective first jaw or second jaw.

Example 113: The method of any example herein, in particular Examples 109-112, wherein the second end portion of the first jaw and the second end portion of the second jaw form an open end of the clip, and the first end portion of the first jaw and the first end portion of the second jaw form a closed end of the clip, and the one or more elongate couplers are configured to engage the expansion apparatus advanced in a direction from the closed end of the clip towards the open end of the clip.

Example 114: The method of any example herein, in particular Examples 109-113, wherein the one or more elongate couplers are configured to slidably engage with the expansion apparatus.

Example 115: The method of any example herein, in particular Examples 106-114, wherein the spring includes a loop extending towards the first end portion of the first jaw and the first end portion of the second jaw.

Example 116: The method of any example herein, in particular Examples 106-115, wherein the first jaw and the second jaw form a compression channel between the first jaw and the second jaw, the compression channel having an opening at the second end portion of the first jaw and the second end portion of the second jaw, and the compression channel being closed at the first end portion of the first jaw and the first end portion of the second jaw.

Example 117: The method of any example herein, in particular Example 116, wherein the spring closes the compression channel at the first end portion of the first jaw and the first end portion of the second jaw.

Example 118: The method of any example herein, in particular Examples 106-117, wherein the first jaw includes: a first end surface positioned at the first end portion of the first jaw, a second end surface positioned at the second end portion of the first jaw, an outer surface facing opposite the first compression surface, a first side surface extending from the first compression surface to the outer surface of the first jaw, and a second side surface extending from the first compression surface to the outer surface of the first jaw and facing opposite the first side surface.

Example 119: The method of any example herein, in particular Example 118, wherein the first side surface is curved concave relative to the first jaw.

Example 120: The method of any example herein, in particular Examples 106-119, wherein the portion of the heart is a left atrial appendage.

Example 121: A clip for a portion of a heart, the clip comprising: a first jaw extending from a first end portion to a second end portion along a length of the first jaw, the first jaw including a first compression surface comprising silicone; a second jaw extending from a first end portion to a second end portion along a length of the second jaw, the second jaw including a second compression surface comprising silicone; and a spring configured to force the first jaw and the second jaw together to compress the portion of the heart between the first compression surface and the second compression surface.

Example 122: The clip of any example herein, in particular Example 121, wherein the silicone of the first compression surface is overmolded upon a base of the first jaw.

Example 123: The clip of any example herein, in particular Example 121 or Example 122, wherein the silicone of the first compression surface is positioned upon fabric.

Example 124: The clip of any example herein, in particular Example 123, wherein the silicone is dip coated upon the fabric.

Example 125: The clip of any example herein, in particular Example 123 or Example 124, wherein the silicone is coated upon fibers forming the fabric.

Example 126: The clip of any example herein, in particular Examples 121-125, wherein the first compression surface is textured.

Example 127: The clip of any example herein, in particular Examples 121-126, wherein the clip includes one or more elongate couplers positioned on one or more of the first jaw or the second jaw and extending along the length of the respective first jaw or second jaw, the one or more elongate couplers configured to engage an expansion apparatus for the clip.

Example 128: The clip of any example herein, in particular Example 127, wherein the one or more elongate couplers comprise one or more channels.

Example 129: The clip of any example herein, in particular Example 127 or

Example 128, wherein the one or more elongate couplers comprise one or more protrusions.

Example 130: The clip of any example herein, in particular Examples 127-129, wherein the second end portion of the first jaw and the second end portion of the second jaw form an open end of the clip, and the first end portion of the first jaw and the first end portion of the second jaw form a closed end of the clip, and the one or more elongate couplers are configured to engage the expansion apparatus advanced in a direction from the closed end of the clip towards the open end of the clip.

Example 131: The clip of any example herein, in particular Examples 121-130, wherein the spring includes a loop extending towards the first end portion of the first jaw and the first end portion of the second jaw.

Example 132: The clip of any example herein, in particular Examples 121-131, wherein the first jaw and the second jaw form a compression channel between the first jaw and the second jaw, the compression channel having an opening at the second end portion of the first jaw and the second end portion of the second jaw, and the compression channel being closed at the first end portion of the first jaw and the first end portion of the second jaw.

Example 133: The clip of any example herein, in particular Example 132, wherein the spring closes the compression channel at the first end portion of the first jaw and the first end portion of the second jaw.

Example 134: The clip of any example herein, in particular Examples 121-133, wherein the first jaw includes: a first end surface positioned at the first end portion of the first jaw, a second end surface positioned at the second end portion of the first jaw, an outer surface facing opposite the first compression surface, a first side surface extending from the first compression surface to the outer surface of the first jaw, and a second side surface extending from the first compression surface to the outer surface of the first jaw and facing opposite the first side surface.

Example 135: The clip of any example herein, in particular Examples 121-134, wherein the clip is configured to occlude a left atrial appendage.

Example 136: A method comprising: deploying a clip to close a portion of a heart, the clip including: a first jaw extending from a first end portion to a second end portion along a length of the first jaw, the first jaw including a first compression surface comprising silicone, a second jaw extending from a first end portion to a second end portion along a length of the second jaw, the second jaw including a second compression surface comprising silicone, and a spring configured to force the first jaw and the second jaw together to compress the portion of the heart between the first compression surface and the second compression surface.

Example 137: The method of any example herein, in particular Example 136, wherein the silicone of the first compression surface is overmolded upon a base of the first jaw.

Example 138: The method of any example herein, in particular Example 136 or Example 137, wherein the silicone of the first compression surface is positioned upon fabric.

Example 139: The method of any example herein, in particular Example 138, wherein the silicone is dip coated upon the fabric.

Example 140: The method of any example herein, in particular Example 138 or Example 139, wherein the silicone is coated upon fibers forming the fabric.

Example 141: The method of any example herein, in particular Examples 136-140, wherein the first compression surface is textured.

Example 142: The method of any example herein, in particular Examples 136-141, wherein the clip includes one or more elongate couplers positioned on one or more of the first jaw or the second jaw and extending along the length of the respective first jaw or second jaw, the one or more elongate couplers configured to engage an expansion apparatus for the clip.

Example 143: The method of any example herein, in particular Example 142, wherein the one or more elongate couplers comprise one or more channels.

Example 144: The method of any example herein, in particular Example 142 or Example 143, wherein the one or more elongate couplers comprise one or more protrusions.

Example 145: The method of any example herein, in particular Examples 142-144, wherein the second end portion of the first jaw and the second end portion of the second jaw form an open end of the clip, and the first end portion of the first jaw and the first end portion of the second jaw form a closed end of the clip, and the one or more elongate couplers are configured to engage the expansion apparatus advanced in a direction from the closed end of the clip towards the open end of the clip.

Example 146: The method of any example herein, in particular Examples 136-

145, wherein the spring includes a loop extending towards the first end portion of the first jaw and the first end portion of the second jaw.

Example 147: The method of any example herein, in particular Examples 136-146, wherein the first jaw and the second jaw form a compression channel between the first jaw and the second jaw, the compression channel having an opening at the second end portion of the first jaw and the second end portion of the second jaw, and the compression channel being closed at the first end portion of the first jaw and the first end portion of the second jaw.

Example 148: The method of any example herein, in particular Example 147, wherein the spring closes the compression channel at the first end portion of the first jaw and the first end portion of the second jaw.

Example 149: The method of any example herein, in particular Examples 136-148, wherein the first jaw includes: a first end surface positioned at the first end portion of the first jaw, a second end surface positioned at the second end portion of the first jaw, an outer surface facing opposite the first compression surface, a first side surface extending from the first compression surface to the outer surface of the first jaw, and a second side surface extending from the first compression surface to the outer surface of the first jaw and facing opposite the first side surface.

Example 150: The method of any example herein, in particular Examples 136-149, wherein the portion of the heart is a left atrial appendage.

Example 151: A system comprising: a spacer device configured to space a first compression surface of a first jaw of a clip for a portion of a heart from a second compression surface of a second jaw of the clip such that the first compression surface and the second compression surface are retained separated from each other.

Example 152: The system of any example herein, in particular Example 151, wherein the spacer device includes an insertion body configured to be positioned between the first compression surface and the second compression surface to space the first compression surface from the second compression surface.

Example 153: The system of any example herein, in particular Example 152, wherein the insertion body is configured to contact the first compression surface and the second compression surface.

Example 154: The system of any example herein, in particular Example 152 or

Example 153, wherein the insertion body includes a planar surface configured to extend parallel with the first compression surface and the second compression surface.

Example 155: The system of any example herein, in particular Examples 151-154, wherein the spacer device includes a removal body for removing the spacer device, thereby removing the spacing of the first compression surface from the second compression surface.

Example 156: The system of any example herein, in particular Example 155, wherein the removal body is configured to be gripped by a user to remove the spacer device.

Example 157: The system of any example herein, in particular Example 155 or Example 156, wherein the spacer device includes an insertion body configured to be positioned between the first compression surface and the second compression surface to space the first compression surface from the second compression surface, the insertion body being a folded substrate, and the removal body includes at least two portions configured to be pulled to unfold the substrate.

Example 158: The system of any example herein, in particular Examples 151-157, wherein the spacer device includes a cover for covering at least one exterior surface of the clip.

Example 159: The system of any example herein, in particular Example 158, wherein the cover is configured to be removed from the at least one exterior surface of the clip.

Example 160: The system of any example herein, in particular Examples 151-159, wherein the spacer device is configured to be removed from the clip in a direction transverse to a longitudinal dimension of the clip.

Example 161: The system of any example herein, in particular Examples 151-160, wherein the spacer device is configured to be removed from the clip along a longitudinal dimension of the clip.

Example 162: The system of any example herein, in particular Examples 151-161, wherein the spacer device is configured to engage at least a portion of an expansion apparatus for the clip to space the first compression surface from the second compression surface.

Example 163: The system of any example herein, in particular Example 162, wherein the spacer device is configured to engage an engagement portion of the expansion apparatus to space the first compression surface from the second compression surface.

Example 164: The system of any example herein, in particular Example 162 or Example 163, wherein the spacer device is configured to separate a first arm of the expansion apparatus for engaging the first jaw from a second arm of the expansion apparatus for engaging the second jaw to space the first compression surface from the second compression surface.

Example 165: The system of any example herein, in particular Example 164, wherein the spacer device is configured to resist a compression force between the first arm and the second arm.

Example 166: The system of any example herein, in particular Example 164 or Example 165, wherein the spacer device includes a first support body for engaging the first arm and a second support body for engaging the second arm, the first support body and the second support body resisting a compression force between the first arm and the second arm.

Example 167: The system of any example herein, in particular Example 166, wherein the first support body is a first rail for sliding relative to the first arm, and the second support body is a second rail for sliding relative to the second arm.

Example 168: The system of any example herein, in particular Examples 162-167, wherein the spacer device includes at least one pin configured to engage an engagement portion of the expansion apparatus.

Example 169: The system of any example herein, in particular Examples 162-168, wherein the spacer device includes a cover for covering at least a portion of an engagement portion of the expansion apparatus.

Example 170: The system of any example herein, in particular Example 169, wherein the cover is a sheath for extending over at least a portion of a first arm of the expansion apparatus for engaging the first jaw and at least a portion of a second arm of the expansion apparatus for engaging the second jaw.

Example 171: The system of any of cl of any example herein, in particular Examples 162-170, wherein the spacer device includes at least one arm for engaging at least a portion of an engagement portion of the expansion apparatus.

Example 172: The system of any example herein, in particular Example 171, wherein the at least one arm includes a first arm and a second arm, the first arm configured to engage an opposite side of the engagement portion of the expansion apparatus than the second arm.

Example 173: The system of any example herein, in particular Example 171 or Example 172, wherein the at least one arm has a first end portion and an opposite second end portion, and the first end portion is configured to engage a first arm of the expansion apparatus for engaging the first jaw, and the second end portion is configured to engage an anchoring portion of the expansion apparatus to resist a compressive force between the first arm and a second arm of the expansion apparatus for engaging the second jaw.

Example 174: The system of any example herein, in particular Examples 151-173, further comprising the clip.

Example 175: The system of any example herein, in particular Example 174, wherein the first compression surface of the first jaw is made of an elastomeric material, and the second compression surface of the second jaw is made of an elastomeric material.

Example 176: The system of any example herein, in particular Example 175, wherein the elastomeric material is silicone.

Example 177: The system of any example herein, in particular Examples 151-176, further comprising an expansion apparatus for the clip.

Example 178: The system of any example herein, in particular Example 177, wherein the expansion apparatus includes a first arm for engaging the first jaw and a second arm for engaging the second jaw.

Example 179: The system of any example herein, in particular Example 177 or Example 178, further comprising the clip, and wherein the clip is held by an engagement portion of the expansion apparatus, and the spacer device spaces the first compression surface of the first jaw from the second compression surface of the second jaw such that the first compression surface and the second compression surface are retained separated from each other.

Example 180: The system of any example herein, in particular Examples 151-179, wherein the clip is for a left atrial appendage (LAA) of the heart.

Example 181: A method comprising: utilizing a spacer device to space a first compression surface of a first jaw of a clip for a portion of a heart from a second compression surface of a second jaw of the clip such that the first compression surface and the second compression surface are retained separated from each other.

Example 182: The method of any example herein, in particular Example 181, wherein the spacer device includes an insertion body configured to be positioned between the first compression surface and the second compression surface to space the first compression surface from the second compression surface.

Example 183: The method of any example herein, in particular Example 182, wherein the insertion body is configured to contact the first compression surface and the second compression surface.

Example 184: The method of any example herein, in particular Example 182 or Example 183, wherein the insertion body includes a planar surface configured to extend parallel with the first compression surface and the second compression surface.

Example 185: The method of any example herein, in particular Examples 181-184, wherein the spacer device includes a removal body for removing the spacer device, thereby removing the spacing of the first compression surface from the second compression surface.

Example 186: The method of any example herein, in particular Example 185, wherein the removal body is configured to be gripped by a user to remove the spacer device.

Example 187: The method of any example herein, in particular Example 185 or Example 186, wherein the spacer device includes an insertion body configured to be positioned between the first compression surface and the second compression surface to space the first compression surface from the second compression surface, the insertion body being a folded substrate, and the removal body includes at least two portions configured to be pulled to unfold the substrate.

Example 188: The method of any example herein, in particular Examples 181-187, wherein the spacer device includes a cover for covering at least one exterior surface of the clip.

Example 189: The method of any example herein, in particular Example 188, wherein the cover is configured to be removed from the at least one exterior surface of the clip.

Example 190: The method of any example herein, in particular Examples 181-189, wherein the spacer device is configured to be removed from the clip in a direction transverse to a longitudinal dimension of the clip.

Example 191: The method of any example herein, in particular Examples 181-

190, wherein the spacer device is configured to be removed from the clip along a longitudinal dimension of the clip.

Example 192: The method of any example herein, in particular Examples 181-

191, wherein the spacer device is configured to engage at least a portion of an expansion apparatus for the clip to space the first compression surface from the second compression surface.

Example 193: The method of any example herein, in particular Example 192, wherein the spacer device is configured to engage an engagement portion of the expansion apparatus to space the first compression surface from the second compression surface.

Example 194: The method of any example herein, in particular Example 192 or Example 193, wherein the spacer device is configured to separate a first arm of the expansion apparatus for engaging the first jaw from a second arm of the expansion apparatus for engaging the second jaw to space the first compression surface from the second compression surface.

Example 195: The method of any example herein, in particular Example 194, wherein the spacer device is configured to resist a compression force between the first arm and the second arm.

Example 196: The method of any example herein, in particular Example 194 or Example 195, wherein the spacer device includes a first support body for engaging the first arm and a second support body for engaging the second arm, the first support body and the second support body resisting a compression force between the first arm and the second arm.

Example 197: The method of any example herein, in particular Example 196, wherein the first support body is a first rail for sliding relative to the first arm, and the second support body is a second rail for sliding relative to the second arm.

Example 198: The method of any example herein, in particular Examples 192-197, wherein the spacer device includes at least one pin configured to engage an engagement portion of the expansion apparatus.

Example 199: The method of any example herein, in particular Examples 192-198, wherein the spacer device includes a cover for covering at least a portion of an engagement portion of the expansion apparatus.

Example 200: The method of any example herein, in particular Example 199, wherein the cover is a sheath for extending over at least a portion of a first arm of the expansion apparatus for engaging the first jaw and at least a portion of a second arm of the expansion apparatus for engaging the second jaw.

Example 201: The method of any example herein, in particular Examples 192-200, wherein the spacer device includes at least one arm for engaging at least a portion of an engagement portion of the expansion apparatus.

Example 202: The method of any example herein, in particular Example 201, wherein the at least one arm includes a first arm and a second arm, the first arm configured to engage an opposite side of the engagement portion of the expansion apparatus than the second arm.

Example 203: The method of any example herein, in particular Example 201 or Example 202, wherein the at least one arm has a first end portion and an opposite second end portion, and the first end portion is configured to engage a first arm of the expansion apparatus for engaging the first jaw, and the second end portion is configured to engage an anchoring portion of the expansion apparatus to resist a compressive force between the first arm and a second arm of the expansion apparatus for engaging the second jaw.

Example 204: The method of any example herein, in particular Examples 181-203, wherein the first compression surface of the first jaw is made of an elastomeric material, and the second compression surface of the second jaw is made of an elastomeric material.

Example 205: The method of any example herein, in particular Example 204, wherein the elastomeric material is silicone.

Example 206: The method of any example herein, in particular Examples 181-205, wherein an expansion apparatus holds the clip and includes a first arm for engaging the first jaw and a second arm for engaging the second jaw.

Example 207: The method of any example herein, in particular Example 206, wherein the clip is held by an engagement portion of the expansion apparatus, and the spacer device spaces a first compression surface of the first jaw from the second compression surface of the second jaw such that the first compression surface and the second compression surface are retained separated from each other.

Example 208: The method of any example herein, in particular Examples 181-207, wherein the clip is for a left atrial appendage (LAA) of the heart.

Example 209: The method of any example herein, in particular Examples 181-208, further comprising removing the spacer device from the clip.

Example 210: The method of any example herein, in particular Examples 181-209, further comprising engaging the spacer device with the clip or with an expansion apparatus for the clip.

Example 211: A sizer for a portion of a heart, the sizer comprising: an elongate shaft including: a central shaft portion having a first end and a second end opposite the first end; a first end portion of the elongate shaft, the first end portion being coupled to the first end of the central shaft portion and extending from the first end of the central shaft portion at a first angle; one or more indicators on the first end portion configured to indicate a size of the portion of the heart; a second end portion of the elongate shaft, the second end portion being coupled to the second end of the central shaft portion and extending from the second end of the central shaft portion at a second angle that is different than the first angle; and one or more indicators on the second end portion configured to indicate a size of the portion of the heart.

Example 212: The sizer of any example herein, in particular Example 211, wherein the central shaft portion is linear.

Example 213: The sizer of any example herein, in particular Example 211 or Example 212, wherein the first end portion is linear.

Example 214: The sizer of any example herein, in particular Examples 211-213, wherein the second end portion is linear.

Example 215: The sizer of any example herein, in particular Examples 211-214, wherein the central shaft portion has a uniform diameter.

Example 216: The sizer of any example herein, in particular Examples 211-215, wherein the first end portion has a uniform diameter and the second end portion has a uniform diameter.

Example 217: The sizer of any example herein, in particular Examples 211-216, wherein the first angle is non-perpendicular and the second angle is non-perpendicular.

Example 218: The sizer of any example herein, in particular Examples 211-217, wherein the first end portion includes a first end and a second end, the first end being coupled to the first end of the central shaft portion, and the one or more indicators on the first end portion are configured to indicate a size of the portion of the heart measured from the second end of the first end portion.

Example 219: The sizer of any example herein, in particular Examples 211-218, wherein the second end portion includes a first end and a second end, the first end being coupled to the second end of the central shaft portion, and the one or more indicators on the second end portion are configured to indicate a size of the portion of the heart measured from the second end of the second end portion.

Example 220: The sizer of any example herein, in particular Examples 211-219, wherein the portion of the heart is a left atrial appendage (LAA).

Example 221: A method comprising: utilizing a sizer to size a portion of a heart, the sizer including: an elongate shaft having: a central shaft portion having a first end and a second end opposite the first end, a first end portion of the elongate shaft, the first end portion being coupled to the first end of the central shaft portion and extending from the first end of the central shaft portion at a first angle, one or more indicators on the first end portion configured to indicate a size of the portion of the heart, a second end portion of the elongate shaft, the second end portion being coupled to the second end of the central shaft portion and extending from the second end of the central shaft portion at a second angle that is different than the first angle, and one or more indicators on the second end portion configured to indicate a size of the portion of the heart.

Example 222: The method of any example herein, in particular Example 221, wherein the central shaft portion is linear.

Example 223: The method of any example herein, in particular Example 221 or Example 222, wherein the first end portion is linear.

Example 224: The method of any example herein, in particular Examples 221-223, wherein the second end portion is linear.

Example 225: The method of any example herein, in particular Examples 221-224, wherein the central shaft portion has a uniform diameter.

Example 226: The method of any example herein, in particular Examples 221-225, wherein the first end portion has a uniform diameter and the second end portion has a uniform diameter.

Example 227: The method of any example herein, in particular Examples 221-226, wherein the first angle is non-perpendicular and the second angle is non-perpendicular.

Example 228: The method of any example herein, in particular Examples 221-227, wherein the first end portion includes a first end and a second end, the first end being coupled to the first end of the central shaft portion, and the one or more indicators on the first end portion are configured to indicate a size of the portion of the heart measured from the second end of the first end portion.

Example 229: The method of any example herein, in particular Examples 221-228, wherein the second end portion includes a first end and a second end, the first end being coupled to the second end of the central shaft portion, and the one or more indicators on the second end portion are configured to indicate a size of the portion of the heart measured from the second end of the second end portion.

Example 230: The method of any example herein, in particular Examples 221-229, wherein the portion of the heart is a left atrial appendage (LAA).

Any of the features of any of the examples, including but not limited to any of the first through 230 examples referred to above, is applicable to all other aspects and examples identified herein, including but not limited to any examples of any of the first through 230 examples referred to above. Moreover, any of the features of an example of the various examples, including but not limited to any examples of any of the first through 230 examples referred to above, is independently combinable, partly or wholly with other examples described herein in any way, e.g., one, two, or three or more examples may be combinable in whole or in part. Further, any of the features of the various examples, including but not limited to any examples of any of the first through 230 examples referred to above, may be made optional to other examples. Any example of a method can be performed by a system or apparatus of another example, and any aspect or example of a system or apparatus can be configured to perform a method of another aspect or example, including but not limited to any examples of any of the first through 230 examples referred to above.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is o (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosure. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. An expansion apparatus for a left atrial appendage clip, the expansion apparatus comprising:
an elongate shaft having a first end portion and a second end portion; and
an engagement portion positioned at the first end portion of the elongate shaft and having at least two arms each extending from a base, the at least two arms including:
a first arm extending longitudinally and for sliding engagement with a first jaw of the left atrial appendage clip and having a proximal end portion fixedly coupled to the base, and
a second arm extending longitudinally and for sliding engagement with a second jaw of the left atrial appendage clip and having a proximal end portion pivotally coupled to the base, the second arm configured to pivot at the base to open or close the left atrial appendage clip.

2. The expansion apparatus of claim 1, wherein the first arm includes at least one rail for sliding engagement with the first jaw, and the second arm includes at least one rail for sliding engagement with the second jaw.

3. The expansion apparatus of claim 1, wherein the first arm includes a channel for receiving at least a portion of the first jaw, and the second arm includes a channel for receiving at least a portion of the second jaw.

4. The expansion apparatus of claim 1, wherein the first arm is configured to move away from the second arm to open the left atrial appendage clip and is configured to move towards the second arm to close the left atrial appendage clip.

5. The expansion apparatus of claim 1, wherein an angle between the first arm and the second arm varies from a closed configuration of the first arm and the second arm to an opened configuration of the first arm and the second arm.

6. The expansion apparatus of claim 1, further comprising a retention mechanism configured to retain the left atrial appendage clip to the engagement portion to impede sliding release of the left atrial appendage clip from the first arm and the second arm.

7. The expansion apparatus of claim 6, wherein the retention mechanism is configured to provide a force to retain the left atrial appendage clip to the engagement portion to impede sliding release of the left atrial appendage clip from the first arm and the second arm.

8. The expansion apparatus of claim 6, wherein the retention mechanism includes a barrier configured to impede sliding release of the left atrial appendage clip from the first arm and the second arm.

9. The expansion apparatus of claim 6, wherein the retention mechanism is configured to release the left atrial appendage clip from the first arm and the second arm in response to a force of occlusion being applied to the left atrial appendage clip.

10. The expansion apparatus of claim 6, wherein the retention mechanism is actuatable.

11. The expansion apparatus of claim 6, wherein the retention mechanism is selectively actuatable from a retention configuration in which the retention mechanism impedes sliding release of the left atrial appendage clip from the first arm and the second arm, to a release configuration in which the retention mechanism allows for sliding release of the left atrial appendage clip from the first arm and the second arm.

12. The expansion apparatus of claim 11, further comprising a control device operable by a user to selectively actuate the retention mechanism.

13. The expansion apparatus of claim 12, wherein the control device comprises one or more of a lever, a slider, or a knob.

14. The expansion apparatus of claim 6, wherein the retention mechanism includes at least one protrusion configured to engage at least a portion of the left atrial appendage clip to impede sliding release of the left atrial appendage clip from the first arm and the second arm.

15. The expansion apparatus of claim 14, wherein the at least one protrusion is selectively actuatable from a retention configuration in which the at least one protrusion impedes sliding release of the left atrial appendage clip from the first arm and the second arm, to a release configuration in which the at least one protrusion allows for sliding release of the left atrial appendage clip from the first arm and the second arm.

16. The expansion apparatus of claim 15, wherein the retention mechanism includes a lever arm and a tether, the at least one protrusion being coupled to the lever arm, and the tether configured to apply a force to the lever arm to actuate the at least one protrusion from the retention configuration to the release configuration.

17. The expansion apparatus of claim 1, further comprising a handle positioned at the second end portion of the elongate shaft.

18. The expansion apparatus of claim 17, further comprising a control mechanism for controlling opening and closing of the first arm relative to the second arm and including a control device positioned upon the handle.

19. The expansion apparatus of claim 1, further comprising a rotation mechanism for controlling rotation of the base about a longitudinal axis of the elongate shaft.

20. The expansion apparatus of claim 19, wherein the rotation mechanism includes a lock for selectively locking a rotational position of the base about the longitudinal axis of the elongate shaft.
